SEQUENCE LISTING (114 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - protein, 25 aa, Artificial Sequence
  TDP-43 peptide
NAGSGSGFNGGFGSSMDSKSSGWGM

SEQ ID NO: 3 - protein, 5 aa, Artificial Sequence
  31F3 CDRH1
GYYLH

SEQ ID NO: 4 - protein, 17 aa, Artificial Sequence
  31F3 CDRH2
RINPGSGGTKYAQKFQG

SEQ ID NO: 5 - protein, 13 aa, Artificial Sequence
  31F3 CDRH3
YQVYGERGRYMDV

SEQ ID NO: 6 - protein, 14 aa, Artificial Sequence
  31F3 CDRL1
TGTSSDVGTYDLVS

SEQ ID NO: 7 - protein, 7 aa, Artificial Sequence
  31F3 CDRL2
EDNKRPS

SEQ ID NO: 8 - protein, 10 aa, Artificial Sequence
  31F3 CDRL3
CSYAAISTLI

SEQ ID NO: 9 - protein, 122 aa, Artificial Sequence
  31F3 VH
QVQLEQSGAEVKRPGASVKVSCKASGYTFTGYYLHWVRQAPGQGLEWMGRINPGSGGTKYAQKFQGRVSMTRDTSISTAYMEVNRLRPDDTAVYYCARYQVYGERGRYMDVWGKGTTVTVSS

SEQ ID NO: 10 - protein, 110 aa, Artificial Sequence
  31F3 VL
QSALTQPASVSGSPGQSITISCTGTSSDVGTYDLVSWYQQHPGKAPKLIIYEDNKRPSGLSNRFSGSKSGNTASLTISGLQADDEADYYCCSYAAISTLIFGGGTKVTVL

SEQ ID NO: 11 - protein, 499 aa, Artificial Sequence
  31F3 heavy chain
QVQLEQSGAEVKRPGASVKVSCKASGYTFTGYYLHWVRQAPGQGLEWMGRINPGSGGTKYAQKFQGRVSMTRDTSISTAYMEVNRLRPDDTAVYYCARYQVYGERGRYMDVWGKGTTVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 12 - protein, 216 aa, Artificial Sequence
  31F3 light chain
QSALTQPASVSGSPGQSITISCTGTSSDVGTYDLVSWYQQHPGKAPKLIIYEDNKRPSGLSNRFSGSKSGNTASLTISGLQADDEADYYCCSYAAISTLIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 5 aa, Artificial Sequence
  30E3 CDRH1
DYYLH

SEQ ID NO: 14 - protein, 17 aa, Artificial Sequence
  30E3 CDRH2
RINPNSGNTNYAQKFQG

SEQ ID NO: 15 - protein, 17 aa, Artificial Sequence
  30E3 CDRH3
VKIVVLRSTPTLYYFDY

SEQ ID NO: 16 - protein, 7 aa, Artificial Sequence
  30E3 CDRL2
EGSKRPS

SEQ ID NO: 17 - protein, 10 aa, Artificial Sequence
  30E3 CDRL3
CSYAGTSTLV

SEQ ID NO: 18 - protein, 126 aa, Artificial Sequence
  30E3 VH
QVQLVQSGAEVKKPGASVKVSCKTSGYTFTDYYLHWVRQAPGQGLEWMGRINPNSGNTNYAQKFQGRVSVTRDTSISTAYMELSRLSSDDTAVYFCARVKIVVLRSTPTLYYFDYWGQGTLVTVSS

SEQ ID NO: 19 - protein, 110 aa, Artificial Sequence
  30E3 VL
QSALTQPASVSGSPGQSITISCTGTSSDVGTYDLVSWYQQHPGKAPKVMIYEGSKRPSGVSNRFSGSKSGNTASLTISGLQAEDEADYYCCSYAGTSTLVFGGGTKVTVL

SEQ ID NO: 20 - protein, 488 aa, Artificial Sequence
  30E3 heavy chain
QVQLVQSGAEVKKPGASVKVSCKTSGYTFTDYYLHWVRQAPGQGLEWMGRINPNSGNTNYAQKFQGRVSVTRDTSISTAYMELSRLSSDDTAVYFCARVKIVVLRSTPTLYYFDYWGQGTLVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 21 - protein, 216 aa, Artificial Sequence
  30E3 light chain
QSALTQPASVSGSPGQSITISCTGTSSDVGTYDLVSWYQQHPGKAPKVMIYEGSKRPSGVSNRFSGSKSGNTASLTISGLQAEDEADYYCCSYAGTSTLVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - protein, 17 aa, Artificial Sequence
  9F11 CDRH2
RINPKSGNTNYAQKFQG

SEQ ID NO: 23 - protein, 17 aa, Artificial Sequence
  9F11 CDRH3
VKIVVVRSTPTLYYLDY

SEQ ID NO: 24 - protein, 14 aa, Artificial Sequence
  9F11 CDRL1
TGSSSDVGTYDLVS

SEQ ID NO: 25 - protein, 7 aa, Artificial Sequence
  9F11 CDRL2
EGHKRPS

SEQ ID NO: 26 - protein, 126 aa, Artificial Sequence
  9F11 VH
QVQLVQSGAEVKKPGASVKVSCKTSGYTFTDYYLHWVRQAPGQGLEWMGRINPKSGNTNYAQKFQGRVTVTRDTSISTAYMELSRLRSDDTAVYYCARVKIVVVRSTPTLYYLDYWGQGTLVTVSS

SEQ ID NO: 27 - protein, 110 aa, Artificial Sequence
  9F11 VL
QSALTQPASVSGSPGQSITISCTGSSSDVGTYDLVSWYQQHPGKAPKVMIYEGHKRPSGVSNRVSGSKSGNTASLTISGLQAEDEADYYCCSYAGTSTLVFGGGTKVTVL

SEQ ID NO: 28 - protein, 5 aa, Artificial Sequence
  15D7 CDRH1
NYAMH

SEQ ID NO: 29 - protein, 17 aa, Artificial Sequence
  15D7 CDRH2
AMSSNGGSTYYGDSVKG

SEQ ID NO: 30 - protein, 13 aa, Artificial Sequence
  15D7 CDRH3
RGRKAPSLYYFDC

SEQ ID NO: 31 - protein, 11 aa, Artificial Sequence
  15D7 CDRL1
RASQSVSSNLA

SEQ ID NO: 32 - protein, 7 aa, Artificial Sequence
  15D7 CDRL2
GASTRAT

SEQ ID NO: 33 - protein, 10 aa, Artificial Sequence [sequence omitted]

SEQ ID NO: 34 - protein, 122 aa, Artificial Sequence
  15D7 VH
EVQLVESGGGLVQPGGSLRLSCSASGFTFGNYAMHWVRQAPGKGLEYVSAMSSNGGSTYYGDSVKGRFTISRDNSKNTLYLQMSSLRAEDTAVYYCVTRGRKAPSLYYFDCWGPGTLVTVSS

SEQ ID NO: 35 - protein, 108 aa, Artificial Sequence
  15D7 VL
EIVLTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTRLEIK

SEQ ID NO: 36 - protein, 17 aa, Artificial Sequence
  CDRH2
RINPNAGNTNYAQKFQG

SEQ ID NO: 37 - protein, 17 aa, Artificial Sequence
  CDRH2
RINPKAGNTNYAQKFQG

SEQ ID NO: 38 - protein, 17 aa, Artificial Sequence
  CDRH2
RINPNSGNVNYAQKFQG

SEQ ID NO: 39 - protein, 17 aa, Artificial Sequence
  CDRH2
RINPNSGNANYAQKFQG

SEQ ID NO: 40 - protein, 17 aa, Artificial Sequence
  CDRH2
RINPKSGNVNYAQKFQG

SEQ ID NO: 41 - protein, 17 aa, Artificial Sequence
  CDRH2
RINPKSGNANYAQKFQG

SEQ ID NO: 42 - protein, 17 aa, Artificial Sequence
  CDRH2
RINPGSGGTRYAQKFQG

SEQ ID NO: 43 - protein, 17 aa, Artificial Sequence
  CDRH2
RINPGSGGTQYAQKFQG

SEQ ID NO: 44 - protein, 17 aa, Artificial Sequence
  CDRH2
RINPGSGGTNYAQKFQG

SEQ ID NO: 45 - protein, 17 aa, Artificial Sequence
  CDRH2
RINPGSGGTKYAQRFQG

SEQ ID NO: 46 - protein, 17 aa, Artificial Sequence
  CDRH2
RINPGSGGTKYAQQFQG

SEQ ID NO: 47 - protein, 17 aa, Artificial Sequence
  CDRH2
RINPNSGNTNYAQRFQG

SEQ ID NO: 48 - protein, 17 aa, Artificial Sequence
  CDRH2
RINPNSGNTNYAQQFQG

SEQ ID NO: 49 - protein, 17 aa, Artificial Sequence
  CDRH2
RINPKSGNTNYAQRFQG

SEQ ID NO: 50 - protein, 17 aa, Artificial Sequence
  CDRH2
RINPKSGNTNYAQQFQG

SEQ ID NO: 51 - protein, 17 aa, Artificial Sequence
  CDRH3
VRIVVLRSTPTLYYFDY

SEQ ID NO: 52 - protein, 17 aa, Artificial Sequence
  CDRH3
VRIVVVRSTPTLYYLDY

SEQ ID NO: 53 - protein, 7 aa, Artificial Sequence
  CDRL2
ELNKRPS

SEQ ID NO: 54 - protein, 7 aa, Artificial Sequence
  CDRL2
EQNKRPS

SEQ ID NO: 55 - protein, 7 aa, Artificial Sequence
  CDRL2
EANKRPS

SEQ ID NO: 56 - protein, 7 aa, Artificial Sequence
  CDRL2
EDNRRPS

SEQ ID NO: 57 - protein, 7 aa, Artificial Sequence
  CDRL2
EDNQRPS

SEQ ID NO: 58 - protein, 7 aa, Artificial Sequence
  CDRL2
EGSRRPS

SEQ ID NO: 59 - protein, 7 aa, Artificial Sequence
  CDRL2
EGSQRPS

SEQ ID NO: 60 - protein, 7 aa, Artificial Sequence
  CDRL2
EGHRRPS

SEQ ID NO: 61 - protein, 7 aa, Artificial Sequence
  CDRL2
EGHQRPS

SEQ ID NO: 62 - protein, 10 aa, Artificial Sequence
  CDRL3
SSYAAISTLI

SEQ ID NO: 63 - protein, 10 aa, Artificial Sequence
  CDRL3
SSYAGTSTLV

SEQ ID NO: 64 - protein, 10 aa, Artificial Sequence
  CDRL3
QSYAAISTLI

SEQ ID NO: 65 - protein, 10 aa, Artificial Sequence
  CDRL3
QSYAGTSTLV

SEQ ID NO: 66 - protein, 10 aa, Artificial Sequence
  CDRL3
GSYAAISTLI

SEQ ID NO: 67 - protein, 10 aa, Artificial Sequence
  CDRL3
GSYAGTSTLV

SEQ ID NO: 68 - protein, 330 aa, Artificial Sequence [sequence omitted]

SEQ ID NO: 69 - protein, 106 aa, Artificial Sequence
  lambda light chain CL
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 70 - protein, 107 aa, Artificial Sequence [sequence omitted]

SEQ ID NO: 71 - protein, 5 aa, Artificial Sequence
  peptide linker motif
GGGGS

SEQ ID NO: 72 - protein, 4 aa, Artificial Sequence
  retention peptide
KDEL

SEQ ID NO: 73 - protein, 7 aa, Artificial Sequence
  nuclear localization sequence
PKKKRKV

SEQ ID NO: 74 - protein, 22 aa, Artificial Sequence
  s3Flag
DYKDHDGDYKDHDIDYKDDDDK

SEQ ID NO: 75 - protein, 9 aa, Artificial Sequence
  HA tag
YPYDVPDYA

SEQ ID NO: 76 - DNA, 15 bp, Artificial Sequence
  31F3 CDRH1
ggctactatttacac

SEQ ID NO: 77 - DNA, 51 bp, Artificial Sequence
  31F3 CDRH2
cggatcaaccctggcagtggtggcacaaagtatgcacagaagtttcagggc

SEQ ID NO: 78 - DNA, 39 bp, Artificial Sequence
  31F3 CDRH3
taccaagtgtacggtgagcgcgggaggtacatggacgtc

SEQ ID NO: 79 - DNA, 42 bp, Artificial Sequence
  31F3 CDRL1
actggaaccagcagtgatgttgggacttatgaccttgtctcc

SEQ ID NO: 80 - protein, 21 aa, Artificial Sequence
  31F3 CDRL2
GAAGACAATAAGCGGCCCTCA

SEQ ID NO: 81 - DNA, 30 bp, Artificial Sequence
  31F3 CDRL3
tgctcatatgctgctattagcactttaata

SEQ ID NO: 82 - DNA, 378 bp, Artificial Sequence
  31F3 VH
caggtgcagctggtgcagtctggggctgaggtgaagaaacctggggcctcagtgaaggtctcctgcaagacttctggatacaccttcaccgactactatttacactgggtgcgacaggcccctggacaagggcttgagtggatgggacggatcaaccctaacagtggtaacacaaactatgcacagaagtttcagggccgggtcagcgtgaccagggacacgtccatcagcacagcctacatggagctgagcaggctgagctctgacgacacggccgtgtatttctgtgcgagagttaagatagtagttctaaggagcacgccaacgctctactactttgactactggggccagggaaccctggtcaccgtgtcctca

SEQ ID NO: 83 - DNA, 330 bp, Artificial Sequence
  31F3 VL
cagtctgccctgactcagcctgcctccgtgtctgggtctcctggacagtcgatcaccatctcctgcactggaaccagcagtgatgttgggacttatgaccttgtctcctggtaccaacagcacccaggcaaagcccccaaactcatcatttatgaagacaataagcggccctcagggctttctaatcgcttctctggctccaagtctggcaacacggcctccctgacgatctctgggctccaggctgacgacgaggctgattattactgctgctcatatgctgctattagcactttaatattcggcggagggaccaaggtgaccgtccta

SEQ ID NO: 84 - DNA, 1624 bp, Artificial Sequence
  31F3 heavy chain
  misc_feature (9)..(10): n is a, c, g, or t
  misc_feature (32)..(32): n is a, c, g, or t
  misc_feature (34)..(34): n is a, c, g, or t
  misc_feature (46)..(46): n is a, c, g, or t
tatcctggnngagggcatggggaatcttcttntngggggcggcagncacacgagcgcactcccaggtgcagctggagcagtctggggctgaggtgaagaggcctggggcctcagtgaaggtctcctgcaaggcttctggatacaccttcaccggctactatttacactgggtgcgacaggcccctggacaaggacttgagtggatgggacggatcaaccctggcagtggtggcacaaagtatgcacagaagtttcagggcagggtcagcatgacccgggacacgtccatcagcacagcctatatggaggtgaacagactgagacctgacgacacggccgtgtattattgtgcgagataccaagtgtacggtgagcgcgggaggtacatggacgtctggggcaaagggaccacggtcaccgtctcgtcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagtgcgacggccggcaagcccccgctccccgggctctcggggtcgcgcgaggatgcttggcac

SEQ ID NO: 85 - DNA, 715 bp, Artificial Sequence
  31F3 light chain
  misc_feature (6)..(6): n is a, c, g, or t
acctcnggcacagggtcctgggcccagtctgccctgactcagcctgcctccgtgtctgggtctcctggacagtcgatcaccatctcctgcactggaaccagcagtgatgttgggacttatgaccttgtctcctggtaccaacagcacccaggcaaagcccccaaactcatcatttatgaagacaataagcggccctcagggctttctaatcgcttctctggctccaagtctggcaacacggcctccctgacgatctctgggctccaggctgacgacgaggctgattattactgctgctcatatgctgctattagcactttaatattcggcggagggaccaaggtgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctaaaccctcaccccccccacgggagactagagctga

SEQ ID NO: 86 - DNA, 15 bp, Artificial Sequence
  30E3 CDRH1
gactactatttacac

SEQ ID NO: 87 - DNA, 51 bp, Artificial Sequence
  30E3 CDRH2
cggatcaaccctaacagtggtaacacaaactatgcacagaagtttcagggc

SEQ ID NO: 88 - DNA, 51 bp, Artificial Sequence
  30E3 CDRH3
gttaagatagtagttctaaggagcacgccaacgctctactactttgactac

SEQ ID NO: 89 - DNA, 42 bp, Artificial Sequence; identical to SEQ ID NO: 79
  30E3 CDRL1

SEQ ID NO: 90 - DNA, 21 bp, Artificial Sequence
  30E3 CDRL2
gagggcagtaagcggccctca

SEQ ID NO: 91 - DNA, 30 bp, Artificial Sequence
  30E3 CDRL3
tgctcatatgcaggtactagcaccttggtg

SEQ ID NO: 92 - DNA, 378 bp, Artificial Sequence; identical to SEQ ID NO: 82
  30E3 VH

SEQ ID NO: 93 - DNA, 330 bp, Artificial Sequence
  30E3 VL
cagtctgccctgactcagcctgcctccgtgtctgggtctcctggacagtcgatcaccatctcctgcactggaaccagcagtgatgttgggacttatgaccttgtctcctggtaccaacaacatccaggcaaagcccccaaagtcatgatttatgagggcagtaagcggccctcaggggtttctaatcgcttctctggctccaagtctggcaacacggcctccctgacaatctccggactccaggctgaggacgaggctgattattactgctgctcatatgcaggtactagcaccttggtgttcggcggagggaccaaggtgaccgtccta

SEQ ID NO: 94 - DNA, 1570 bp, Artificial Sequence
  30E3 heavy chain
atcctcttcttggtggcagcagccacaggagcccactcccaggtgcagctggtgcagtctggggctgaggtgaagaaacctggggcctcagtgaaggtctcctgcaagacttctggatacaccttcaccgactactatttacactgggtgcgacaggcccctggacaagggcttgagtggatgggacggatcaaccctaacagtggtaacacaaactatgcacagaagtttcagggccgggtcagcgtgaccagggacacgtccatcagcacagcctacatggagctgagcaggctgagctctgacgacacggccgtgtatttctgtgcgagagttaagatagtagttctaaggagcacgccaacgctctactactttgactactggggccagggaaccctggtcaccgtgtcctcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagtgcgacggccggcaagcccccgctccccgggctctcggggtcgcgcgaggatgcttggcacgt

SEQ ID NO: 95 - DNA, 728 bp, Artificial Sequence
  30E3 light chain
cactcagggcacagggtcctgggcccagtctgccctgactcagcctgcctccgtgtctgggtctcctggacagtcgatcaccatctcctgcactggaaccagcagtgatgttgggacttatgaccttgtctcctggtaccaacaacatccaggcaaagcccccaaagtcatgatttatgagggcagtaagcggccctcaggggtttctaatcgcttctctggctccaagtctggcaacacggcctccctgacaatctccggactccaggctgaggacgaggctgattattactgctgctcatatgcaggtactagcaccttggtgttcggcggagggaccaaggtgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttcataggttctcaaccctcaccccccaccacgggagactagagctgcaggatcccagg

SEQ ID NO: 96 - DNA, 51 bp, Artificial Sequence
  9F11 CDRH2
cggatcaaccctaagagtggtaacacaaactatgcacagaaatttcagggc

SEQ ID NO: 97 - DNA, 51 bp, Artificial Sequence
  9F11 CDRH3
gttaagatagtagtggtaaggagcacgccaaccctctactaccttgactac

SEQ ID NO: 98 - DNA, 42 bp, Artificial Sequence
  9F11 CDRL1
actggatccagcagtgatgttgggacttatgaccttgtctcc

SEQ ID NO: 99 - DNA, 21 bp, Artificial Sequence
  9F11 CDRL2
gagggccataagcggccctca

SEQ ID NO: 100 - DNA, 378 bp, Artificial Sequence
  9F11 VH
caggtgcagctggtgcagtctggggctgaggtgaagaaacctggggcctcagtgaaggtctcctgcaagacttctggatacaccttcaccgactactatttacactgggtgcgacaggcccctggacaagggcttgagtggatgggacggatcaaccctaagagtggtaacacaaactatgcacagaaatttcagggccgggtcaccgtgaccagggacacgtccatcagcacagcctacatggagctgagcaggttgagatctgacgacacggccgtgtattattgtgcgagagttaagatagtagtggtaaggagcacgccaaccctctactaccttgactactggggccagggaaccctggtcaccgtgtcctca

SEQ ID NO: 101 - DNA, 330 bp, Artificial Sequence
  9F11 VL
cagtctgccctgactcagcctgcctccgtgtctgggtctcctggacagtcgatcaccatctcctgcactggatccagcagtgatgttgggacttatgaccttgtctcctggtaccaacagcacccaggcaaagcccccaaggtcatgatttatgagggccataagcggccctcaggggtttctaatcgcgtctctggctccaagtctggcaacacggcctccctgacaatctccgggctccaggctgaggacgaggctgattattactgctgctcatatgcaggtactagcaccttggtgttcggcggagggaccaaggtgaccgtccta

SEQ ID NO: 102 - DNA, 15 bp, Artificial Sequence
  15D7 CDRH1
aactatgctatgcac

SEQ ID NO: 103 - DNA, 51 bp, Artificial Sequence
  15D7 CDRH2
gctatgagtagtaatgggggtagcacatactacggagactccgtgaagggc

SEQ ID NO: 104 - DNA, 39 bp, Artificial Sequence
  15D7 CDRH3
cggggtagaaaggcgccttcgctttactactttgactgc

SEQ ID NO: 105 - DNA, 33 bp, Artificial Sequence
  15D7 CDRL1
agggccagtcagagtgttagcagcaacttagcc

SEQ ID NO: 106 - DNA, 21 bp, Artificial Sequence
  15D7 CDRL2
ggtgcatccaccagggccacc

SEQ ID NO: 107 - DNA, 30 bp, Artificial Sequence
  15D7 CDRL3
cagcagtataataactggcctccgtggacc

SEQ ID NO: 108 - DNA, 366 bp, Artificial Sequence
  15D7 VH
gaggtgcagctggtggagtctgggggaggcttggtccagcctggggggtccctgagactctcctgttcagcctctggattcaccttcggtaactatgctatgcactgggtccgccaggctccagggaagggactggaatatgtttcagctatgagtagtaatgggggtagcacatactacggagactccgtgaagggcagattcaccatctccagagacaattccaagaacacgttgtatcttcaaatgagcagtctgagagctgaggacacggctgtttattactgtgtgacgcggggtagaaaggcgccttcgctttactactttgactgctggggcccgggaaccctggtcaccgtctcctca

SEQ ID NO: 109 - DNA, 324 bp, Artificial Sequence
  15D7 VL
gaaattgtgttgacacagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccaccggtatcccagccaggttcagtggcagtgggtctgggacagagttcactctcaccatcagcagcctgcagtctgaagattttgcagtttattactgtcagcagtataataactggcctccgtggaccttcggccaagggacacgactggagattaaa

SEQ ID NO: 110 - protein, 413 aa, Artificial Sequence
  TDP-43 variant
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 111 - protein, 122 aa, Artificial Sequence
  31F3 Hf VH
QVQLVQSGAEVKKPGASVKVSCKASGYTFTGYYLHWVRQAPGQGLEWMGRINPGSGGTKYAQKFQGRVTMTRDTSISTAYMELSRLRSDDTAVYYCARYQVYGERGRYMDVWGKGTTVTVSS

SEQ ID NO: 112 - protein, 110 aa, Artificial Sequence
  31F3 Lf VL
QSALTQPASVSGSPGQSITISCTGTSSDVGTYDLVSWYQQHPGKAPKLMIYEDNKRPSGVSNRFSGSKSGNTASLTISGLQAEDEADYYCCSYAAISTLIFGSGTKVTVL

SEQ ID NO: 113 - protein, 122 aa, Artificial Sequence
  31F3 Hg VH
QVQLVQSGAEVKRPGASVKVSCKASGYTFTGYYLHWVRQAPGQGLEWMGRINPGSGGTKYAQKFQGRVTMTRDTSISTAYMELSRLRSDDTAVYYCARYQVYGERGRYMDVWGKGTTVTVSS

SEQ ID NO: 114 - protein, 110 aa, Artificial Sequence
  31F3 Lg VL
QSALTQPASVSGSPGQSITISCTGTSSDVGTYDLVSWYQQHPGKAPKLIIYEDNKRPSGVSNRFSGSKSGNTASLTISGLQAEDEADYYCCSYAAISTLIFGSGTKVTVL

(19) United States
(12) Patent Application Publication
(10) Pub. No.: US 2024/0150449 A1
(43) Pub. Date: May 9, 2024

(54) ANTIBODIES AGAINST TDP-43 AND METHODS OF USING THE SAME

(71) Applicants: MABYLON AG, Schlieren (CH); UNIVERSITY OF ZURICH, Zürich (CH)

(72) Inventors: Dimitri BIELI, Basel (CH); Natascha WUILLEMIN, Zürich (CH); Niccolo PENGO, Adliswil (CH); Tiziana Maria SONATI, Zürich (CH); Pierre DE ROSSI, Zürich (CH); Magdalini POLYMENIDOU, Zürich (CH); Marc EMMENEGGER, Fehraltorf (CH); Adriano AGUZZI, Zürich (CH)

(21) Appl. No.: 18/549,629

(22) PCT Filed: Mar. 10, 2022
(86) PCT No.: PCT/EP2022/056162

§ 371 Date: Sep. 8, 2023

(30) Foreign Application Priority Data
Mar. 10, 2021 (WO) PCT/EP2021/056040

(51) Int. Cl. C07K 16/18; A61K 39/00; A61K 49/00; A61P 25/28; G01N 33/68

(52) U.S. Cl. CPC C07K 16/18; A61K 49/0002; A61P 25/28; G01N 33/6896; A61K 2039/505; C07K 2317/21; C07K 2317/622

(57) ABSTRACT

The present invention relates to antibodies, and antigen binding fragments thereof, that specifically bind to the pathological form of TDP-43, which characterizes various TDP-43 proteinopathies. The antibodies, and antigen binding fragments thereof, decrease TDP-43 aggregation observed under pathological conditions. The invention also relates to nucleic acids that encode, and to cells that express such antibodies and antibody fragments. In addition, the invention relates to the use of the antibodies and antibody fragments in the diagnosis, treatment and prevention of TDP-43 proteinopathies.

The present invention relates to the field of TDP-43 proteinopathies, in particular to antibodies binding pathological TDP-43.

Amyotrophic lateral sclerosis (ALS), a disease in which premature loss of upper and lower motor neurons leads to fatal paralysis, is the most common motor neuron disease in adults. Frontotemporal lobar degeneration (FTLD), a neurodegenerative disorder characterized by behavioral and language dysfunction, is the most common dementia under the age of 60.

Nevertheless, there are no effective therapies for these neurodegenerative disorders and management focuses on treating the symptoms and providing palliative care in order to improve the quality of life of these patients. Besides palliative care, there are currently only two approved medications for ALS: Riluzole, which increases life expectancy only up to six months and the recently FDA-approved Radicava, which showed promising effects in decreasing symptoms, but no effect on survival has been demonstrated yet. Similarly, in FTLD, antidepressants and antipsychotics are prescribed to reduce behavioral symptoms, but do not modify disease progression, demonstrating the urgent need for development of effective, targeted therapies for these devastating diseases.

ALS is now increasingly recognized to have clinical, genetic and pathological overlap with FTLD (Neary, D. et al. Frontotemporal lobar degeneration: a consensus on clinical diagnostic criteria. Neurology 51, 1546-54 (1998)). In 2006, TAR DNA-binding protein 43 (TDP-43) was identified as the major component of ubiquitinated cytoplasmic inclusions observed in both ALS and FTLD patients (Arai, T. et al. TDP-43 is a component of ubiquitin-positive tau-negative inclusions in frontotemporal lobar degeneration and amyotrophic lateral sclerosis. Biochem. Biophys. Res. Commun. 351, 602-611 (2006); Neumann, M. et al. Ubiquitinated TDP-43 in frontotemporal lobar degeneration and amyotrophic lateral sclerosis. Science (80-.). 314, 130-133 (2006)). Dominant mutations in the gene TARDBP were subsequently identified as a primary cause of ALS (Gitcho, M. A. et al. TDP-43 A315T mutation in familial motor neuron disease. Ann Neurol. 63, 535-538 (2008); Kabashi, E. et al. TARDBP mutations in individuals with sporadic and familial amyotrophic lateral sclerosis. Nat Genet 40, 572-574 (2008); Sreedharan, J. et al. TDP-43 mutations in familial and sporadic amyotrophic lateral sclerosis. Science (80-.). 319, 1668-1672 (2008)). Affected brains and spinal cords present a biochemical signature characterized by abnormal hyperphosphorylation and ubiquitination of TDP-43. TDP-43 pathology characterizes most instances of ALS and FTLD, and its spatiotemporal manifestation coincides with neurodegeneration, supporting its central pathogenic role in these diseases.

With the seminal discovery that the RNA binding protein TDP-43 is the major component of the cytoplasmic ubiquitinated inclusions found in sporadic ALS and FTLD patients, researchers obtained, for the first time, a handle with which to investigate sporadic disease (Lagier-Tourenne, C., Polymenidou, M. & Cleveland, D. W. TDP-43 and FUS/TLS: emerging roles in RNA processing and neurodegeneration. Hum. Mol. Genet. 19, R46-R64 (2010)). Indeed, TDP-43 inclusions are now recognized as the pathological hallmark correlating with neurodegeneration in several conditions including the vast majority (>95%) of sporadic ALS patients, a significant percentage (45%) of patients with FTLD (Ling, S.-C., Polymenidou, M. & Cleveland, D. W. Converging Mechanisms in ALS and FTD: Disrupted RNA and Protein Homeostasis. Neuron 79, 416-438 (2013)) as well as ˜30% of Alzheimer's patients (Amador-Ortiz, C. et al. TDP-43 immunoreactivity in hippocampal sclerosis and Alzheimer's disease. Ann. Neurol. 61, 435-445 (2007); Higashi, S. et al. Concurrence of TDP-43, tau and α-synuclein pathology in brains of Alzheimer's disease and dementia with Lewy bodies. Brain Res. 1184, 284-294 (2007); Hu, W. T. et al. Temporal lobar predominance of TDP-43 neuronal cytoplasmic inclusions in Alzheimer's disease. Acta Neuropathol. 116, 215-220 (2008)).

While TDP-43 is arguably the most important molecular target for ALS and FTLD therapy, its essential cellular functions preclude therapeutic strategies reducing the overall level of the protein. TDP-43, a highly conserved and ubiquitously expressed protein belonging to the heterogeneous nuclear ribonucleoprotein (hnRNP) family, was shown to bind both DNA and RNA. TDP-43 has multiple functions in transcriptional repression, pre-mRNA splicing and translational regulation. TDP-43 is pivotal in multiple cellular functions including regulation of RNA metabolism, mRNA transport, microRNA maturation and stress granule formation. Under normal physiological conditions, TDP-43 localizes predominantly in the cell nucleus.

Human TDP-43 has a length of 414 amino acids and contains an N-terminal domain (NTD) spanning residues 1-76; two highly conserved folded RNA recognition motifs spanning residues 106-176 (RRM1) and 191-259 (RRM2), which are required to bind target RNA and DNA; and an unstructured C-terminal domain (CTD) encompassing residues 274-414 (CTD), which contains a glycine-rich region and which is involved in protein-protein interactions.

An increasing number of reports suggest that TDP-43 can behave in a prion-like manner, both in in vitro and in vivo experimental systems (Brettschneider, J. et al. TDP-43 pathology and neuronal loss in amyotrophic lateral sclerosis spinal cord. Acta Neuropathol. 128, 423-437 (2014); Brettschneider, J. et al. Stages of pTDP-43 pathology in amyotrophic lateral sclerosis. Ann. Neurol. 74, 20-38 (2013); Furukawa, Y., Kaneko, K., Watanabe, S., Yamanaka, K. & Nukina, N. A seeding reaction recapitulates intracellular formation of sarkosyl-insoluble transactivation response element (TAR) DNA-binding protein-43 inclusions. J. Biol. Chem. 286, 18664-18672 (2011); Nonaka, T. et al. Prion-like Properties of Pathological TDP-43 Aggregates from Diseased Brains. Cell Rep. 4, 124-134 (2013); Porta, S. et al. Patient-derived frontotemporal lobar degeneration brain extracts induce formation and spreading of TDP-43 pathology in vivo. Nat. Commun. 9, (2018)). Misfolded proteins released from a cell harboring pathological inclusions can be transmitted to recipient cells and act as templates for the subsequent misfolding of native proteins. The reiteration of this process leads to cell-to-cell propagation of pathological proteins throughout the brain (Porta, S. et al. Patient-derived frontotemporal lobar degeneration brain extracts induce formation and spreading of TDP-43 pathology in vivo. Nat. Commun. 9, (2018); Aguzzi, A. & Rajendran, L. The Transcellular Spread of Cytosolic Amyloids, Prions, and Prionoids. Neuron 64, 783-790 (2009); Polymenidou, M. & Cleveland, D. W. Prion-like spread of protein aggregates in neurodegeneration. J. Exp. Med 209, 889-893 (2012); Polymenidou, M. & Cleveland, D. W. The seeds of neurodegeneration: prion-like spreading in ALS. Cell 147, 498-508 (2011)). This model of propagation and neuroanatomic spread provides a molecular mechanism for disease progression. Most importantly, this mechanism indicates that extracellular forms of aggregated TDP-43 seeds exist. In disease, aggregation of TDP-43 in the cytoplasm happens concomitantly with loss of nuclear localization of TDP-43 (Arai, T. et al. TDP-43 is a component of ubiquitin-positive tau-negative inclusions in frontotemporal lobar degeneration and amyotrophic lateral sclerosis. Biochem. Biophys. Res. Commun. 351, 602-611 (2006); Neumann, M. et al. Ubiquitinated TDP-43 in frontotemporal lobar degeneration and amyotrophic lateral sclerosis. Science. 2006 Oct. 6; 314(5796): 130-3. doi: 10.1126/science.1134108). Both loss of nuclear function and accumulation of toxic aggregates are thought to be involved in the process of neurodegeneration.

While immunotherapies against various types of cancer are a proven potent therapeutic approach, immunotherapies against neurodegenerative diseases are still at their early stages of development. However, they hold tremendous potential due to their direct impact on the underlying disease biology and their potential to delay disease progression. Targeting cell-to-cell propagation with specific antibodies would potentially be treating the root of disease by containing the disease in the primo-affected area. Naturally occurring human monoclonal antibodies represent novel therapeutic molecules for neurologic disorders including ALS (Wootla, B. et al. Naturally Occurring Monoclonal Antibodies and Their Therapeutic Potential for Neurologic Diseases. JAMA Neurol. 1-8 (2015). doi:10.1001/jamaneurol.2015.2188). It is postulated that human autoantibodies targeting misfolded pathogenic proteins serve as surveillance molecules to eliminate toxic aggregates before they can elicit a deleterious response (Szabo, P., Relkin, N. & Weksler, M. E. Natural human antibodies to amyloid beta peptide. Autoimmunity Reviews 7, 415-420 (2008)). Such antibodies might neutralize the activity of oligomers and/or facilitate the clearance of deposited aggregates via microglia uptake.

Antibodies derived from non-human sources, such as chimeric or humanized antibodies, can show an adverse immune response, which can lead to nausea, diarrhea and flu-like symptoms (Lu, Z.-J. Frontier of therapeutic antibody discovery: The challenges and how to face them. World J. Biol. Chem. 3, 187 (2012)). In more severe cases, these side-effects can be lethal. Additionally, non-human antibody segments can also reduce the efficacy of the therapeutic antibody, as they can be neutralized by the human immune system. However, antibodies retrieved from individuals with no debilitating conditions have a potentially higher safety profile, as the antibody has proven tolerability in the human body. Combined with the outstanding affinity maturation typical of the immune system, human antibodies are likely to offer a therapeutic window superior to immunoreagents derived from conventional antibody sources or synthetic libraries, such as antibodies of non-human origin.

In view of the above, it is the object of the present invention to overcome the drawbacks of the prior art. In particular, it is an object of the present invention to provide antibodies, which bind specifically to a pathological form of TDP-43, which is involved in various diseases. It is also an object of the present invention to provide antibodies, which provide protection from pathological TDP-43 aggregation. Human antibodies usually have reduced or no immunogenicity compared to antibodies retrieved from non-human sources.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x+20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the subject or patient is a human.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “binding” and similar reference usually means “specifically binding”, which does not encompass non-specific sticking. In particular, specific binding of an antibody means that the antibody recognizes its target antigen and binds its target with greater affinity (or at lower antibody concentrations, e.g. EC50) than it does to a structurally different antigen and/or to an antigen with a modified or mutated sequence. Thereby, a “greater” affinity may be at least 2fold, 3fold, 4fold, 5fold, 10fold, 15fold, 20fold, 25fold, 50fold, 75fold, 100fold 150fold, 200fold, 500fold, 750fold, 1,000fold, 1,500fold, 2,000fold, 5,000fold, 7,500fold, 10,000fold or even higher affinity as compared to the binding to a control antigen. In some instances, antibody-binding to the control antigen may be undetectable (below detection threshold), while antibody-binding to the specific antigen may be well detected/determined.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (e.g., variant or mutant antibodies) as long as the characteristic properties according to the invention are retained. In some embodiments, the antibody is a human antibody. In some embodiments, the antibody is a monoclonal antibody. For example, the antibody may be a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multi-specific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice or chicken) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As used herein, the expression “human antibodies” includes non-naturally occurring sequence variants of human antibodies, which are usually obtained by introducing one or more mutations in the (naturally occurring) human antibodies. Such mutations include one or more mutations in a CDR or in a framework region, as well as Fc modifications (e.g., as known in the art for specific functionalities).

As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1 or igG4. Antibodies of the invention may have a κ or a λ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### SUMMARY

In particular, the present invention provides the following items:
1. An antibody, or an antigen-binding fragment thereof, which binds to a phosphorylated C-terminal domain or fragment of TAR-DNA-binding protein 43 kDa (TDP-43; SEQ ID NO: 1 or 110) comprising amino acids 391-414 of SEQ ID NO: 1 or 110, wherein the serine residues at positions 403 and/or 404 of SEQ ID NO: 1 or 110 are phosphorylated.
2. The antibody, or an antigen-binding fragment thereof, according to item 1, wherein the antibody or the antigen-binding fragment comprises:
   (i) a CDRH1 according to General Formula I:

X₁YYX₂H (I)

wherein
X₁ may be any amino acid; preferably X₁ is a small amino acid selected from the group consisting of G, A, S, C, N, D, T, V and P; more preferably X₁ is G or D; and
   X₂ may be any amino acid; preferably X₂ is a nonpolar amino acid selected from the group consisting of A, C, G, I, L, M, F, P, W and V; more preferably X₂ is M or L; even more preferably X₂ is L;
(ii) a CDRH2 according to General Formula II:

RINPX₁X₂GX₃X₄X₅YAQX₆FQG (II)

wherein
X₁ may be any amino acid; preferably X₁ is N, K or G;
X₂ may be any amino acid; preferably X₂ is a small amino acid selected from the group consisting of G, A, S, C, N, D, T, V and P; more preferably X₂ is S or A; particularly preferably X₂ is S;
X₃ may be any amino acid; preferably X₃ is a small amino acid selected from the group consisting of G, A, S, C, N, D, T, V and P; more preferably X₃ is G or N;
X₄ may be any amino acid; preferably X₄ is a small amino acid selected from the group consisting of G, A, S, C, N, D, T, V and P, or a hydrophobic amino acid selected from the group consisting of A, T, P, C, V, R, F, Y, W, M, I and L; more preferably X₄ is a small hydrophobic amino acid selected from the group consisting of A, T, P, C, and V; even more preferably X₄ is T, V or A; particularly preferably X₄ is T;
X₅ may be any amino acid; preferably X₅ is a polar amino acid selected from the group consisting of N, K, R, Q, S, T, D, E, H, Y and W; more preferably X₅ is N, K, R or Q; particularly preferably X₅ is K or N; and
X₆ may be any amino acid; preferably X₆ is a polar amino acid selected from the group consisting of N, K, R, Q, S, T, D, E, H, Y and W; more preferably X₆ is K, R or Q; particularly preferably X₆ is K;
(iii) a CDRH3 according to SEQ ID NO: 5 or according to General Formula III:

VX₁IVVLRSTPTLYYX₂DY (III)

wherein
X₁ may be any amino acid; preferably X₁ is M, K or R; more preferably X₁ is K or R; even more preferably X₁ is K; and
X₂ may be any amino acid; preferably X₂ is a hydrophobic amino acid selected from the group consisting of A, T, P, C, V, R, F, Y, W, M, I and L; more preferably X₂ is F or L;
(iv) a CDRL1 according to General Formula IV:

TGX₁SSDVGX₂YX₃LVS (IV)

wherein
X₁ may be any amino acid; preferably X₁ is a small amino acid selected from the group consisting of G, A, S, C, N, D, T, V and P or a polar amino acid selected from the group consisting of N, K, R, Q, S, T, D, E, H, Y and W; more preferably X₁ is a small polar amino acid selected from the group consisting of S, T, D or N; even more preferably X₁ is S or T;
X₂ may be any amino acid; preferably X₂ is a small amino acid selected from the group consisting of G, A, S, C, N, D, T, V and P or a polar amino acid selected from the group consisting of N, K, R, Q, S, T, D, E, H, Y and W; more preferably X₂ is a small polar amino acid selected from the group consisting of S, T, D or N; even more preferably X₂ is T or S; particularly preferably X₂ is T; and
X₃ may be any amino acid; preferably X₃ is a small amino acid selected from the group consisting of G, A, S, C, N, D, T, V and P or a polar amino acid selected from the group consisting of N, K, R, Q, S, T, D, E, H, Y and W; more preferably X₃ is a small polar amino acid selected from the group consisting of S, T, D or N; even more preferably X₃ is D or N; particularly preferably X₃ is D;
(v) a CDRL2 according to General Formula V:

EX₁X₂X₃RPS (V)

wherein
X₁ may be any amino acid; preferably X₁ is D, L, Q, A or G or a small amino acid selected from the group consisting of G, A, S, C, N, D, T, V and P; more preferably X₁ is D or G;
X₂ may be any amino acid; preferably X₂ is a polar amino acid selected from the group consisting of N, K, R, Q, S, T, D, E, H, Y and W; more preferably X₂ is N, S or H; and
X₃ may be any amino acid; preferably X₃ is a polar amino acid selected from the group consisting of N, K, R, Q, S, T, D, E, H, Y and W; more preferably X₃ is K, R or Q; particularly preferably X₃ is K;
and
(vi) a CDRL3 according to General Formula VI:

X₁SYAX₂X₃STX₄X₅ (VI)

wherein
X₁ may be any amino acid; preferably X₁ is C, S, Q, or G or a small amino acid selected from the group consisting of G, A, S, C, N, D, T, V and P; more preferably X₁ is C;
X₂ may be any amino acid; preferably X₂ is a small amino acid selected from the group consisting of G, A, S, C, N, D, T, V and P or an aliphatic non-polar amino acid selected from the group consisting of A, G, I, L and V; more preferably X₂ is a small aliphatic non-polar amino acid selected from the group consisting of A, G and V; even more preferably X₂ is A or G;
X₃ may be any amino acid; preferably X₃ is I, S or T; more preferably X₃ is I or T;
X₄ may be any amino acid; preferably X₄ is a hydrophobic amino acid selected from the group consisting of A, T, P, C, V, R, F, Y, W, M, I and L; more preferably X₄ is L or W; even more preferably X₄ is L; and
X₅ may be any amino acid; preferably X₅ is an aliphatic non-polar amino acid selected from the group consisting of A, G, I, L and V; more preferably X₅ is I or V.
3. The antibody, or an antigen-binding fragment thereof, according to any one of the previous items, wherein the antibody or the antigen-binding fragment comprises:
   (i) CDRH1, CDRH2, CDRH3, CDRL1, CDRL2 and CDRL3 sequences according to SEQ ID NOs 3-8, respectively;
   (ii) CDRH1, CDRH2, CDRH3, CDRL1, CDRL2 and CDRL3 sequences according to SEQ ID NOs 13, 14, 15, 6, 16 and 17, respectively;
   (iii) CDRH1, CDRH2, CDRH3, CDRL1, CDRL2 and CDRL3 sequences according to SEQ ID NOs 13, 22, 23, 24, 25 and 17, respectively; or
   (iv) CDRH1, CDRH2, CDRH3, CDRL1, CDRL2 and CDRL3 sequences according to SEQ ID NOs 28-33, respectively.
4. An antibody, or an antigen-binding fragment thereof, comprising:
   (i) a CDRH1 according to General Formula I:

X₁YYX₂H (I)

wherein
X₁ may be any amino acid; preferably X₁ is a small amino acid selected from the group consisting of G, A, S, C, N, D, T, V and P; more preferably X₁ is G or D; and
X₂ may be any amino acid; preferably X₂ is a nonpolar amino acid selected from the group consisting of A, C, G, I, L, M, F, P, W and V; more preferably X₂ is M or L; even more preferably X₂ is L;
(ii) a CDRH2 according to General Formula II:

RINPX₁X₂GX₃X₄X₅YAQX₆FQG (II)

wherein
X₁ may be any amino acid; preferably X₁ is N, K or G;
X₂ may be any amino acid; preferably X₂ is a small amino acid selected from the group consisting of G, A, S, C, N, D, T, V and P; more preferably X₂ is S or A; particularly preferably X₂ is S;
X₃ may be any amino acid; preferably X₃ is a small amino acid selected from the group consisting of G, A, S, C, N, D, T, V and P; more preferably X₃ is G or N;
X₄ may be any amino acid; preferably X₄ is a small amino acid selected from the group consisting of G, A, S, C, N, D, T, V and P, or a hydrophobic amino acid selected from the group consisting of A, T, P, C, V, R, F, Y, W, M, I and L; more preferably X₄ is a small hydrophobic amino acid selected from the group consisting of A, T, P, C, and V; even more preferably X₄ is T, V or A; particularly preferably X₄ is T;
X₅ may be any amino acid; preferably X₅ is a polar amino acid selected from the group consisting of N, K, R, Q, S, T, D, E, H, Y and W; more preferably X₅ is N, K, R or Q; particularly preferably X₅ is K or N; and
X₆ may be any amino acid; preferably X₆ is a polar amino acid selected from the group consisting of N, K, R, Q, S, T, D, E, H, Y and W; more preferably X₆ is K, R or Q; particularly preferably X₆ is K;
(iii) a CDRH3 according to SEQ ID NO: 5 or according to General Formula III:

VX₁IVVLRSTPTLYYX₂DY (III)

wherein
X₁ may be any amino acid; preferably X₁ is M, K or R; more preferably X₁ is K or R; even more preferably X₁ is K; and
X₂ may be any amino acid; preferably X₂ is a hydrophobic amino acid selected from the group consisting of A, T, P, C, V, R, F, Y, W, M, I and L; more preferably X₂ is F or L;
(iv) a CDRL1 according to General Formula IV:

TGX₁SSDVGX₂YX₃LVS (IV)

wherein
X₁ may be any amino acid; preferably X₁ is a small amino acid selected from the group consisting of G, A, S, C, N, D, T, V and P or a polar amino acid selected from the group consisting of N, K, R, Q, S, T, D, E, H, Y and W; more preferably X₁ is a small polar amino acid selected from the group consisting of S, T, D or N; even more preferably X₁ is S or T;
X₂ may be any amino acid; preferably X₂ is a small amino acid selected from the group consisting of G, A, S, C, N, D, T, V and P or a polar amino acid selected from the group consisting of N, K, R, Q, S, T, D, E, H, Y and W; more preferably X₂ is a small polar amino acid selected from the group consisting of S, T, D or N; even more preferably X₂ is T or S; particularly preferably X₂ is T; and
X₃ may be any amino acid; preferably X₃ is a small amino acid selected from the group consisting of G, A, S, C, N, D, T, V and P or a polar amino acid selected from the group consisting of N, K, R, Q, S, T, D, E, H, Y and W; more preferably X₃ is a small polar amino acid selected from the group consisting of S, T, D or N; even more preferably X₃ is D or N; particularly preferably X₃ is D;
   (v) a CDRL2 according to General Formula V:

EX₁X₂X₃RPS (V)

wherein
X₁ may be any amino acid; preferably X₁ is D, L, Q, A or G or a small amino acid selected from the group consisting of G, A, S, C, N, D, T, V and P; more preferably X₁ is D or G;
X₂ may be any amino acid; preferably X₂ is a polar amino acid selected from the group consisting of N, K, R, Q, S, T, D, E, H, Y and W; more preferably X₂ is N, S or H; and
X₃ may be any amino acid; preferably X₃ is a polar amino acid selected from the group consisting of N, K, R, Q, S, T, D, E, H, Y and W; more preferably X₃ is K, R or Q; particularly preferably X₃ is K;
   and
   (vi) a CDRL3 according to General Formula VI:

X₁SYAX₂X₃STX₄X₅ (VI)

wherein
X₁ may be any amino acid; preferably X₁ is C, S, Q, or G or a small amino acid selected from the group consisting of G, A, S, C, N, D, T, V and P; more preferably X₁ is C;
X₂ may be any amino acid; preferably X₂ is a small amino acid selected from the group consisting of G, A, S, C, N, D, T, V and P or an aliphatic non-polar amino acid selected from the group consisting of A, G, I, L and V; more preferably X₂ is a small aliphatic non-polar amino acid selected from the group consisting of A, G and V; even more preferably X₂ is A or G;
X₃ may be any amino acid; preferably X₃ is I, S or T; more preferably X₃ is I or T;
X₄ may be any amino acid; preferably X₄ is a hydrophobic amino acid selected from the group consisting of A, T, P, C, V, R, F, Y, W, M, I and L; more preferably X₄ is L or W; even more preferably X₄ is L; and
X₅ may be any amino acid; preferably X₅ is an aliphatic non-polar amino acid selected from the group consisting of A, G, I, L and V; more preferably X₅ is I or V.
5. The antibody, or an antigen-binding fragment thereof, according to any one of the previous items, wherein the antibody or the antigen-binding fragment comprises:
   (i) a CDRH1 according to General Formula I:

X₁YYX₂H (I)

wherein
X₁ is G or D; and
X₂ is L;
(ii) a CDRH2 according to General Formula II:

RINPX₁X₂GX₃X₄X₅YAQX₆FQG (II)

wherein
X₁ is N, K or G;
X₂ is S;
X₃ is G or N;
X₄ is T;
X₅ is K or N; and
X₆ is K;
(iii) a CDRH3 according to SEQ ID NO: 5 or according to General Formula III:

VX₁IVVLRSTPTLYYX₂DY (III)

wherein
X₁ is K; and
X₂ is F or L;
(iv) a CDRL1 according to General Formula IV:

TGX₁SSDVGX₂YX₃LVS (IV)

wherein
X₁ is S or T;
X₂ is T; and
X₃ is D;
(v) a CDRL2 according to General Formula V:

EX₁X₂X₃RPS (V)

wherein
X₁ is D or G;
X₂ is N, S or H; and
X₃ is K;
and
(vi) a CDRL3 according to General Formula VI:

X₁SYAX₂X₃STX₄X₅ (VI)

wherein
X₁ is C;
X₂ is A or G;
X₃ is I or T;
X₄ is L; and
X₅ is I or V.
6. The antibody, or an antigen-binding fragment thereof, according to any one of the previous items, wherein the antibody or the antigen-binding fragment comprises:
   (i) CDRH1, CDRH2, CDRH3, CDRL1, CDRL2 and CDRL3 sequences according to SEQ ID NOs 3-8, respectively;
   (ii) CDRH1, CDRH2, CDRH3, CDRL1, CDRL2 and CDRL3 sequences according to SEQ ID NOs 13, 14, 15, 6, 16 and 17, respectively;
   (iii) CDRH1, CDRH2, CDRH3, CDRL1, CDRL2 and CDRL3 sequences according to SEQ ID NOs 13, 22, 23, 24, 25 and 17, respectively; or
   (iv) CDRH1, CDRH2, CDRH3, CDRL1, CDRL2 and CDRL3 sequences according to SEQ ID NOs 28-33, respectively.
7. An antibody, or an antigen-binding fragment thereof, comprising:
   (i) CDRH1, CDRH2, CDRH3, CDRL1, CDRL2 and CDRL3 sequences according to SEQ ID NOs 3-8, respectively;
   (ii) CDRH1, CDRH2, CDRH3, CDRL1, CDRL2 and CDRL3 sequences according to SEQ ID NOs 13, 14, 15, 6, 16 and 17, respectively;
   (iii) CDRH1, CDRH2, CDRH3, CDRL1, CDRL2 and CDRL3 sequences according to SEQ ID NOs 13, 22, 23, 24, 25 and 17, respectively; or
   (iv) CDRH1, CDRH2, CDRH3, CDRL1, CDRL2 and CDRL3 sequences according to SEQ ID NOs 28-33, respectively.
8. The antibody, or an antigen-binding fragment thereof, according to any one of items 4-7, wherein the antibody or the antigen-binding fragment binds to a phosphorylated C-terminal domain or fragment of TDP-43 (SEQ ID NO: 1 or 110) comprising amino acids 391-414 of SEQ ID NO: 1 or 110, wherein the serine residues at positions 403 and/or 404 of SEQ ID NO: 1 or 110 are phosphorylated.
9. The antibody, or an antigen-binding fragment thereof, according to any one of the previous items, wherein the antibody or the antigen-binding fragment binds to a phosphorylated C-terminal domain or fragment of TDP-43 (SEQ ID NO: 1 or 110) comprising amino acids 391-414 of SEQ ID NO: 1 or 110, wherein the serine residues at positions 403 and 404, but not the serine residues at positions 409 and 410, of SEQ ID NO: 1 or 110 are phosphorylated.
10. The antibody, or an antigen-binding fragment thereof, according to any one of the previous items, wherein the antibody or the antigen-binding fragment does not bind to a phosphorylated C-terminal domain or fragment of TDP-43 (SEQ ID NO: 1 or 110) comprising amino acids 391-414 of SEQ ID NO: 1 or 110, wherein the serine residues at positions 403 and 404 of SEQ ID NO: 1 or 110 are not phosphorylated, preferably the antibody or the antigen-binding fragment does not bind to non-phosphorylated TDP-43.
11. The antibody, or an antigen-binding fragment thereof, according to any one of the previous items, wherein the antibody or the antigen-binding fragment does not bind to a phosphorylated C-terminal domain or fragment of TDP-43 (SEQ ID NO: 1 or 110) comprising amino acids 391-414 of SEQ ID NO: 1 or 110, wherein the serine residues at positions 409 and 410 of SEQ ID NO: 1 or 110 are phosphorylated.
12. The antibody, or an antigen-binding fragment thereof, according to any one of the previous items, wherein the antibody or the antigen-binding fragment binds to a high-molecular weight aggregate of TDP-43.
13. The antibody, or an antigen-binding fragment thereof, according to any one of the previous items, wherein the antibody or the antigen-binding fragment binds to C-terminal fragments of TDP-43.
14. The antibody, or an antigen-binding fragment thereof, according to any one of the previous items, wherein the antibody or the antigen-binding fragment binds to cytoplasmic or neuritic TDP-43 in human CNS tissue.
15. The antibody, or an antigen-binding fragment thereof, according to any one of the previous items, wherein the antibody or the antigen-binding fragment decreases TDP-43 aggregation.
16. The antibody, or an antigen-binding fragment thereof, according to any one of the previous items, wherein the antibody or the antigen-binding fragment comprises (i) a heavy chain variable region comprising an amino acid sequence having at least 70% sequence identity to SEQ ID NO: 9 and a light chain variable region comprising an amino acid sequence having at least 70% sequence identity to SEQ ID NO: 10; or (ii) a heavy chain variable region comprising an amino acid sequence having at least 70% sequence identity to SEQ ID NO: 18 and a light chain variable region comprising an amino acid sequence having at least 70% sequence identity to SEQ ID NO: 19; or (iii) a heavy chain variable region comprising an amino acid sequence having at least 70% sequence identity to SEQ ID NO: 26 and a light chain variable region comprising an amino acid sequence having at least 70% sequence identity to SEQ ID NO: 27; or (iv) a heavy chain variable region comprising an amino acid sequence having at least 70% sequence identity to SEQ ID NO: 34 and a light chain variable region comprising an amino acid sequence having at least 70% sequence identity to SEQ ID NO: 35.
17. The antibody, or an antigen-binding fragment thereof, according to any one of the previous items, wherein the antibody or the antigen-binding fragment comprises (i) a heavy chain variable region comprising an amino acid sequence according to SEQ ID NO: 9 and a light chain variable region comprising an amino acid sequence according to SEQ ID NO: 10; or (ii) a heavy chain variable region comprising an amino acid sequence according to SEQ ID NO: 18 and a light chain variable region comprising an amino acid sequence according to SEQ ID NO: 19; or (iii) a heavy chain variable region comprising an amino acid sequence according to SEQ ID NO: 26 and a light chain variable region comprising an amino acid sequence according to SEQ ID NO: 27; or (iv) a heavy chain variable region comprising an amino acid sequence according to SEQ ID NO: 34 and a light chain variable region comprising an amino acid sequence according to SEQ ID NO: 35.
18. The antibody, or an antigen-binding fragment thereof, according to any one of the previous items, wherein the antibody or the antigen-binding fragment thereof is a human antibody.
19. The antibody, or an antigen-binding fragment thereof, according to any one of the previous items, wherein the antibody, or an antigen-binding fragment thereof, is a monoclonal antibody.
20. The antibody according to any one of the previous items, wherein the antibody comprises an Fc moiety.
21. The antibody according to any one of the previous items, wherein the antibody is of the IgG type.
22. The antibody according to item 21, wherein the antibody is of the IgG1 or IgG4 type.
23. The antibody, or an antigen-binding fragment thereof, according to any one of the previous items, wherein the antibody, or the antigen-binding fragment thereof, is purified.
24. The antibody, or an antigen-binding fragment thereof, according to any one of the previous items, wherein the antibody, or the antigen-binding fragment thereof, is a single-chain antibody, such as an scFv.
25. The antibody, or an antigen-binding fragment thereof, according to any one of the previous items, wherein the antigen-binding fragment is selected from Fab, Fab′, F(ab′)2 and Fv.
26. The antibody, or an antigen-binding fragment thereof, according to any one of the previous items, wherein the antibody, or the antigen-binding fragment thereof, is an intrabody.
27. The antibody, or an antigen-binding fragment thereof, according to any one of the previous items for use as a medicament.
28. The antibody, or an antigen-binding fragment thereof, for use according to item 27 in prophylaxis or treatment of TDP-43 proteinopathy.
29. A nucleic acid molecule comprising a polynucleotide encoding the antibody, or an antigen-binding fragment thereof, according any one of items 1 to 26.
30. The nucleic acid molecule according to item 29, wherein the polynucleotide encoding the antibody, or an antigen-binding fragment thereof, is codon-optimized.
31. The nucleic acid molecule according to item 29 or 30 comprising a nucleic acid sequence as set forth in any one of SEQ ID NOs 76-109; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.
32. The nucleic acid molecule according to any one of items 29-31, wherein the encoded antibody, or an antigen-binding fragment thereof, is an intrabody.
33. A combination of a first and a second nucleic acid molecule, wherein the first nucleic acid molecule comprises a polynucleotide encoding the heavy chain of the antibody, or an antigen-binding fragment thereof, according to any one of items 1 to 26; and the second nucleic acid molecule comprises a polynucleotide encoding the corresponding light chain of the same antibody, or the same antigen-binding fragment thereof.
34. The combination of nucleic acid molecules according to item 33, wherein one or both of the polynucleotides encoding the heavy and/or light chain(s) of the antibody, or an antigen-binding fragment thereof, is/are codon-optimized.
35. The combination of nucleic acid molecules according to item 33 or 34 comprising a nucleic acid sequence as set forth in any one of SEQ ID NOs 76-109; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.
36. A vector comprising the nucleic acid molecule of any one of items 29 to 32 or the combination of nucleic acid molecules of any one of items 33 to 35.
37. A combination of a first and a second vector, wherein the first vector comprises a first nucleic acid molecule as defined in any one of items 33 to 35 and the second vector comprises the corresponding second nucleic acid molecule as defined in any one of items 33 to 35.
38. A host cell expressing the antibody, or an antigen-binding fragment thereof, according to any one of items 1 to 26, or comprising the vector according to item 36 or the combination of vectors of item 37.
39. A method for preparing the antibody, or an antigen-binding fragment thereof, according to any one of items 1 to 26, or immunoglobulin chain(s) thereof, said method comprising
   (i) culturing the host cell according to item 38; and
   (ii) isolating the antibody or immunoglobulin chain(s) thereof from the culture.
40. An immunoconjugate comprising the antibody, or an antigen-binding fragment thereof, according to any one of items 1 to 26 and a detectable label and/or a transport moiety.
41. A composition comprising the antibody, or an antigen-binding fragment thereof, according to any one of items 1 to 26, the nucleic acid according to any one of items 29 to 32, the combination of nucleic acids according to any one of items 33 to 35, the vector according to item 36, the combination of vectors according to item 37, the cell according to item 38 or the immunoconjugate according to item 40.
42. The composition according to item 41, wherein the composition is a pharmaceutical composition and, optionally, further comprises a pharmaceutically acceptable excipient, diluent or carrier.
43. The composition according to item 41, wherein the composition is a diagnostic composition.
44. The antibody, or an antigen-binding fragment thereof, according to any one of items 1 to 26, the nucleic acid according to any one of items 29 to 32, the combination of nucleic acids according to any one of items 33 to 35, the vector according to item 36, the combination of vectors according to item 37, the host cell according to item 38, the immunoconjugate according to item 40, or the pharmaceutical composition according to item 42 for use as a medicament; optionally in the prophylaxis or treatment of a TDP-43 proteinopathy.
45. Use of the antibody, or an antigen-binding fragment thereof, according to any one of items 1 to 26 or of the immunoconjugate according to item 40 in (in-vitro) diagnosis of a TDP-43 proteinopathy.
46. Use of the antibody, or an antigen-binding fragment thereof, according to any one of items 1 to 26 or of the immunoconjugate according to item 40 in a method for detecting pathological TDP-43.
47. Use of the antibody, or an antigen-binding fragment thereof, according to any one of items 1 to 26 in the manufacture of an immunogenic composition comprising a phosphorylated C-terminal domain or fragment of TDP-43 (SEQ ID NO: 1 or 110) comprising amino acids 391-414 of SEQ ID NO: 1 or 110, wherein the serine residues at positions 403 and 404 of SEQ ID NO: 1 or 110 are phosphorylated, in particular for monitoring the quality of the immunogenic composition.
48. Use of the antibody, or an antigen-binding fragment thereof, according to any one of items 1 to 26, the nucleic acid according to any one of items 29 to 32, the combination of nucleic acids according to any one of items 33 to 35, the vector according to item 36, the combination of vectors according to item 37, the cell according to item 38, the immunoconjugate according to item 40, or the pharmaceutical composition according to item 42 in the manufacture of a medicament for prophylaxis, treatment or attenuation of a TDP-43 proteinopathy.
49. A method of reducing a TDP-43 proteinopathy, or lowering the risk of a TDP-43 proteinopathy, comprising: administering to a subject in need thereof, a therapeutically effective amount of the antibody, or an antigen-binding fragment thereof, according to any one of items 1 to 26, the nucleic acid according to any one of items 29 to 32, the combination of nucleic acids according to any one of items 33 to 35, the vector according to item 36, the combination of vectors according to item 37, the host cell according to item 38, the immunoconjugate according to item 40, or the pharmaceutical composition according to item 42.
50. A kit comprising one or more containers comprising one or more of
   (i) the antibody, or an antigen-binding fragment thereof, according to any one of items 1 to 22,
   (ii) the nucleic acid molecule(s) according to any one of items 25 to 31,
   (iii) the vector(s) according to item 32 or 33,
   (iv) the cell according to item 34,
   (v) the immunoconjugate according to item 35; and/or
   (vi) the composition according to any one of items 37 to 39.

### DETAILED DESCRIPTION

Antibodies and Antigen-Binding Fragments Thereof.

In a first aspect the present invention provides an (isolated) antibody, or an antigen-binding fragment thereof, which (specifically) binds to a phosphorylated C-terminal domain or fragment of TAR-DNA-binding protein 43 kDa (TDP-43; SEQ ID NO: 1) comprising amino acids 391-414 of SEQ ID NO: 1, wherein the serine residues at positions 403 and 404 of SEQ ID NO: 1 are phosphorylated.

The present inventors surprisingly identified human antibodies, which specifically bind to pathological, but not to physiological (non-pathological), TDP-43 with high affinity. In particular, these antibodies (specifically) bind to a phosphorylated C-terminal domain or fragment of TAR-DNA-binding protein 43 kDa (TDP-43; SEQ ID NO: 1) comprising amino acids 391-414 of SEQ ID NO: 1, wherein the serine residues at positions 403 and 404 of SEQ ID NO: 1 are phosphorylated. The antibodies were found to bind to high-molecular weight aggregates and C-terminal fragments of TDP-43, which characterize TDP-43 proteinopathies. The pathological forms of TDP-43, to which the antibodies bind to, are found in various diseases including FTLD, ALS, Alzheimer's disease and limbic-predominant age-related TDP-43 encephalopathy (LATE). A bottleneck in the development of effective therapies against ALS and FTLD is the lack of models with pathological features faithfully resembling those present in patient brains for preclinical evaluation of candidate antibodies. In view thereof, the inventors established a new cellular model of TDP-43 aggregation via the introduction of pathological forms extracted directly from patient brains, leading to accumulation and propagation of pathology in cell lines via a mechanism mimicking the molecular events leading to disease progression. The antibodies according to the invention were found to have protective properties in this newly established model, resulting in decreased TDP-43 aggregates.

The antibodies, or an antigen-binding fragment thereof, of the invention (specifically) bind to a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated. Moreover, the antibodies, or an antigen-binding fragment thereof, of the invention (specifically) bind to full-length TDP-43 (SEQ ID NO: 1), wherein the serine residues at positions 403 and 404 of SEQ ID NO: 1 are phosphorylated. In other words, the antibodies bind to (a C-terminal domain of) full-length TDP-43 or to C-terminal fragments thereof, which comprises amino acids 391-414 of SEQ ID NO: 1, wherein the serine residues at positions 403 and 404 (but not the serine residues at positions 409 and 410) of SEQ ID NO: 1 are phosphorylated. SEQ ID NO: 1 shows the sequence of human TDP-43. In particular, the antibodies of the present invention (specifically) bind to (an epitope contained in) amino acids 391-414 of SEQ ID NO: 1, wherein the serine residues at positions 403 and 404 (but not the serine residues at positions 409 and 410) of SEQ ID NO: 1 are phosphorylated.

In particular, phosphorylation of the serine residues at positions 403 and 404 of SEQ ID NO: 1 may result in increased binding (e.g., increased binding affinity) of the antibody as compared to C-terminal domains or fragments of TDP-43, wherein the serine residues at positions 403 and 404 of SEQ ID NO: 1 are not phosphorylated. In some embodiments, the antibodies, or an antigen-binding fragment thereof, do not specifically bind to a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are not phosphorylated. In some instances, the antibody or the antigen-binding fragment does not bind to non-phosphorylated TDP-43.

Accordingly, the antibodies, or an antigen-binding fragment thereof, of the invention (specifically) bind to and recognize TDP-43 (SEQ ID NO: 1) phosphorylated at the serine residues at positions 403 and 404 of SEQ ID NO: 1. In particular, this specific binding of the antibodies is independent from phosphorylation of serine residues at positions 409 and 410 of SEQ ID NO: 1. In some embodiments, the antibodies of the present invention recognize (bind to) TDP-43 phosphorylated at the serine residues at positions 403 and 404, but not phosphorylated at the serine residues at positions 409 and 410. In some embodiments, the antibodies of the present invention recognize (bind to) TDP-43 phosphorylated at the serine residues at positions 403, 404, 409 and 410.

In some embodiments, the antibodies, or an antigen-binding fragment thereof, do not bind to a phosphorylated C-terminal domain or fragment of TDP-43 (SEQ ID NO: 1) comprising amino acids 391-414 of SEQ ID NO: 1, wherein the serine residues at positions 409 and 410 of SEQ ID NO: 1, but not at positions 403 and 404, are phosphorylated.

In some embodiments, the antibodies, or an antigen-binding fragment thereof, of the invention can bind to TDP-43 phosphorylated at the serine residues at positions 403, 404, 409 and 410 simultaneously/concomitantly with an antibody binding to the phosphorylated serine residues at positions 409 and 410. Examples of antibodies binding to the phosphorylated serine residues at positions 409 and 410 include, but are not limited to, anti pS409/410 TDP-43 antibody 66318-1-Ig (Proteintech®). Accordingly, the antibodies, or an antigen-binding fragment thereof, of the invention may bind to a distinct, non-overlapping epitope of pTDP-43 as compared to antibodies binding to the phosphorylated serine residues at positions 409 and 410, such as anti pS409/410 TDP-43 antibody 66318-1-Ig (Proteintech®). Therefore, the antibodies, or an antigen-binding fragment thereof, of the invention may be useful for a combination with an antibody binding to the phosphorylated serine residues at positions 409 and 410. Such a combination enables, for example, the (simultaneous) detection of different phosphorylation patterns of TDP-43.

In some embodiments, the antibodies, or antigen-binding fragments thereof, according to the invention (specifically) bind to a C-terminal domain or fragment of TDP-43 comprising SEQ ID NO: 2 (which corresponds to amino acids from position 391 to 414 of full-length human TDP-43 of SEQ ID NO: 1), which is phosphorylated at the serine residues at positions 14 and 15 (but not at positions 20 and 21) of SEQ ID NO: 2, which correspond to the serine residues at positions 403 and 404 of SEQ ID NO: 1. The antibodies, or antigen-binding fragments thereof, may not (specifically) bind to a C-terminal domain or fragment of TDP-43 comprising SEQ ID NO: 2, which is phosphorylated at the serine residues at positions 20 and 21 (but not at positions 14 and 15) of SEQ ID NO: 2, which correspond to the serine residues at positions 409 and 410 of SEQ ID NO: 1.

Phosphorylation of TDP-43 at the serine residues at positions 403 and 404 has been previously linked with TDP-43 proteinopathies, including FTLD-TDP and ALS. In particular, phosphorylation of TDP-43 at the serine residues at positions 403 and 404 may cause structural changes of full-length TDP-43 that promote intracellular aggregation of TDP-43 (Nonaka T, Suzuki G, Tanaka Y, et al. Phosphorylation of TAR DNA-binding Protein of 43 kDa (TDP-43) by Truncated Casein Kinase 16 Triggers Mislocalization and Accumulation of TDP-43. J Biol Chem. 2016; 291(11):5473-5483. doi:10.1074/jbc.M115.695379), which is observed in TDP-43 proteinopathies.

Accordingly, the antibody, or the antigen-binding fragment, of the present invention preferably binds (specifically) to pathological TDP-43, while it preferably does not (specifically) bind to non-pathological TDP-43. Accordingly, the antibody may be selective for pathological TDP-43. In particular, the antibody or the antigen-binding fragment binds to cytoplasmic or neuritic TDP-43 in human central nervous system (CNS) tissue—but preferably not to nuclear (non-pathological) TDP-43. Accordingly, the antibody or the antigen-binding fragment preferably binds to cytoplasmic or neuritic TDP-43 in human brain or spinal cord tissue. Thus, the antibody, or the antigen-binding fragment, generally specifically recognizes (pathological) TDP-43. For example, the antibodies of the invention specifically recognize pathologic human TDP-43 in a Western Blot, in an ELISA and/or in immunohistochemistry, e.g. of human brain or spinal cord tissue.

As used herein, the term “pathologic TDP-43” refers to extracellular, cytoplasmic and neuritic TDP-43, as well as to “TDP-43 oligomers”, “TDP-43 inclusion bodies” and “TDP-43 (high molecular weight) aggregates” wherein TDP-43 forms fibril-like clumps. Abnormal species of TDP-43 (pathological TDP-43) often migrates with a higher molecular mass at approximately 45 kDa and/or a smear of high-molecular-mass proteins. Moreover, pathologic TDP-43 is usually hyperphosphorylated and ubiquitinated. TDP-43 has been found to exhibit multiple phosphorylation sites in carboxyl-terminal regions of deposited TDP-43 and it is suggested that phosphorylation leads to increased oligomerization and fibrillization of TDP-43. Pathological TDP-43 also includes (phosphorylated)C-terminal fragments (CTFs).

Under pathological conditions abnormal cleavage of TDP-43 results in phosphorylated C-terminal fragments (CTFs). Accumulated CTFs can be found in neural tissue of ALS and FTLD patients. In particular, in the brain of patients with ALS and FTLD, usually TDP-43 CTFs of 25 kDa (CTF-25) can be detected, although—less frequently—also CTFs of approximately 15 and 35 kDa (CTF-35) can be found. The antibody or the antigen-binding fragment may thus (specifically) bind to C-terminal fragments of TDP-43, such as CTF-25.

Aggregation of TDP-43 and spreading of the TDP-43 aggregates are suggested to account for the pathogenesis and progression of TDP-43 proteinopathies (and the related neurodegenerative diseases including FTLD and ALS. Accordingly, the antibody, or the antigen-binding fragment, preferably binds (specifically) to high-molecular weight aggregates of TDP-43. Moreover, the antibody or the antigen-binding fragment may decrease TDP-43 aggregation, in particular TDP-43 aggregation induced by human pathological TDP-43 (from human CNS tissue of patients suffering from a TDP-43 proteinopathy). The antibody can decrease the level, amount or concentration of TDP-43 aggregation by at least about 5%, 10%, 20%, 30%, 40%, 50%, 60%, 70%, 80%, 90% or more. In some embodiments, the antibody or the antigen-binding fragment of the present invention may be capable of reducing the amount or concentration of cytoplasmic TDP-43 protein in the brain or spinal cord, for example, by at least about 5%, 10%, 20%, 30%, 40%, 50%, 60%, 70%, 80%, 90% or more. The % reduction or decrease may be relative compared to the level, number, frequency, amount or concentration that existed before treatment, or to the level, number, frequency, amount or concentration that, exist in an untreated or control-treated subject (e.g., a placebo-treated subject).

In some embodiments, the binding affinity of the antibody, or the antigen-binding fragment thereof, to pS403/404-TDP-43 is very high. In some embodiments, the affinity of the antibody of the present invention, or the antigen-binding fragment thereof, to pS403/404-TDP-43 is higher than the affinity of a polyclonal rabbit antibody obtained as described in EP 2 189 526 A1 or in Hasegawa et al., 2008 (Hasegawa M, Arai T, Nonaka T, Kametani F, Yoshida M, Hashizume Y, Beach T G, Buratti E, Baralle F, Morita M, Nakano I, Oda T, Tsuchiya K, Akiyama H. Phosphorylated TDP-43 in frontotemporal lobar degeneration and amyotrophic lateral sclerosis. Ann Neurol. 2008 Jun. 10; 64(1):60-70). In some embodiments, the affinity of the antibody of the present invention, or the antigen-binding fragment thereof, to pS403/404-TDP-43 is higher than the affinity of the polyclonal rabbit antibody CosmoBio, Catalog No. TIP-PTDP-P05.

Standard methods to assess binding of the antibody according to the present invention, or the antigen-binding fragment thereof, are known to those skilled in the art and include, for example, ELISA (enzyme-linked immunosorbent assay). Thereby, the relative affinities of antibody binding may be determined by measuring the concentration of the antibody (EC₅₀) required to achieve 50% maximal binding at saturation.

An exemplary standard ELISA may be performed as follows: ELISA plates may be coated with a sufficient amount (e.g., 0.1-10 μg/ml) of the peptide (or compound/particle) to which binding of the antibody is to be tested (e.g., TDP-43). Plates may then be incubated with the antibody to be tested. After washing, antibody binding can be revealed, e.g. using a labelled antibody recognizing the test antibody, such as anti-human IgG coupled to HRP (or another label). Plates may then be washed, the required substrate (e.g., a tetramethylbenzidine substrate solution) may be added and plates may be read, e.g. at 450 nm. The relative affinities of antibody binding may be determined by measuring the concentration of the antibody (EC₅₀) required to achieve 50% maximal binding at saturation. The EC₅₀ values may be calculated by interpolation of binding curves fitted with a four-parameter nonlinear regression with a variable slope. To compare the affinity of different antibodies, their EC₅₀ values may be compared.

In some embodiments, the EC₅₀ value obtained with a standard ELISA as described herein for the antibody, or the antigen-binding fragment thereof, of the present invention to pS403/404-TDP-43 may be lower (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% lower) as compared to the EC₅₀ value obtained with a polyclonal rabbit antibody obtained as described in EP 2 189 526 A1 or in Hasegawa et al., 2008 (or as compared to the EC₅₀ value obtained with the polyclonal rabbit antibody CosmoBio, Catalog No. TIP-PTDP-P05).

The present invention also provides an (isolated) antibody, or an antigen-binding fragment thereof, which (specifically) binds to a phosphorylated C-terminal domain or fragment of TDP-43 comprising amino acids 391-414 of SEQ ID NO: 110, wherein the serine residues at positions 403 and 404 of SEQ ID NO: 110 are phosphorylated. In some embodiments, the antibodies of the present invention (specifically) bind to (an epitope contained in) amino acids 391-414 of SEQ ID NO: 110, wherein the serine residues at positions 403 and 404 (but not the serine residues at positions 409 and 410) of SEQ ID NO: 110 are phosphorylated. In some embodiments, the antibodies, or an antigen-binding fragment thereof, do not specifically bind to a phosphorylated C-terminal domain or fragment of TDP-43 (SEQ ID NO: 1) comprising amino acids 391-414 of SEQ ID NO: 110, wherein the serine residues at positions 403 and 404 of SEQ ID NO: 110 are not phosphorylated. The detailed embodiments of the antibodies of the invention as described above and in the following apply accordingly to these antibodies.

In general, the antibodies, or antigen-binding fragments thereof, may comprise three complementarity determining regions (CDRs) in a heavy chain variable region (VH), e.g. on a heavy chain; and three CDRs in a light chain variable region (VL), e.g. on a light chain. In general, complementarity determining regions (CDRs) are the hypervariable regions present in heavy chain variable domains and light chain variable domains. Typically, the CDRs of a heavy chain and the connected light chain variable region of an antibody together form the antigen receptor. Usually, the three CDRs (CDR1, CDR2, and CDR3) are arranged non-consecutively in the variable domain. Since antigen receptors are typically composed of two variable domains (e.g., on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classic IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's.

The sequences of the heavy chains and light chains of exemplary antibodies comprising three different heavy chain CDRs and three different light chain CDRs were determined. The position of the CDR amino acids were defined according to the Kabat numbering system.

In some embodiments, the antibody or the antigen-binding fragment comprises:
(i) a CDRH1 according to General Formula I:

X₁YYX₂H (I)

wherein
X₁ may be any amino acid; preferably X₁ is a small amino acid selected from the group consisting of G, A, S, C, N, D, T, V and P; more preferably X₁ is G or D; and
X₂ may be any amino acid; preferably X₂ is a nonpolar amino acid selected from the group consisting of A, C, G, I, L, M, F, P, W and V; more preferably X₂ is M or L; even more preferably X₂ is L;
(ii) a CDRH2 according to General Formula II:

RINPX₁X₂GX₃X₄X₅YAQX₆FQG (II)

wherein
X₁ may be any amino acid; preferably X₁ is N, K or G;
X₂ may be any amino acid; preferably X₂ is a small amino acid selected from the group consisting of G, A, S, C, N, D, T, V and P; more preferably X₂ is S or A; particularly preferably X₂ is S;
X₃ may be any amino acid; preferably X₃ is a small amino acid selected from the group consisting of G, A, S, C, N, D, T, V and P; more preferably X₃ is G or N;
X₄ may be any amino acid; preferably X₄ is a small amino acid selected from the group consisting of G, A, S, C, N, D, T, V and P, or a hydrophobic amino acid selected from the group consisting of A, T, P, C, V, R, F, Y, W, M, I and L; more preferably X₄ is a small hydrophobic amino acid selected from the group consisting of A, T, P, C, and V; even more preferably X₄ is T, V or A; particularly preferably X₄ is T;
X₅ may be any amino acid; preferably X₅ is a polar amino acid selected from the group consisting of N, K, R, Q, S, T, D, E, H, Y and W; more preferably X₅ is N, K, R or Q; particularly preferably X₅ is K or N; and
X₆ may be any amino acid; preferably X₆ is a polar amino acid selected from the group consisting of N, K, R, Q, S, T, D, E, H, Y and W; more preferably X₆ is K, R or Q; particularly preferably X₆ is K;
(iii) a CDRH3 according to SEQ ID NO: 5 or according to General Formula III:

VX₁IVVLRSTPTLYYX₂DY (III)

wherein
X₁ may be any amino acid; preferably X₁ is M, K or R; more preferably X₁ is K or R; even more preferably X₁ is K; and
X₂ may be any amino acid; preferably X₂ is a hydrophobic amino acid selected from the group consisting of A, T, P, C, V, R, F, Y, W, M, I and L; more preferably X₂ is F or L;
(iv) a CDRL1 according to General Formula IV:

TGX₁SSDVGX₂YX₃LVS (IV)

wherein
X₁ may be any amino acid; preferably X₁ is a small amino acid selected from the group consisting of G, A, S, C, N, D, T, V and P or a polar amino acid selected from the group consisting of N, K, R, Q, S, T, D, E, H, Y and W; more preferably
X₁ is a small polar amino acid selected from the group consisting of S, T, D or N; even more preferably X₁ is S or T;
X₂ may be any amino acid; preferably X₂ is a small amino acid selected from the group consisting of G, A, S, C, N, D, T, V and P or a polar amino acid selected from the group consisting of N, K, R, Q, S, T, D, E, H, Y and W; more preferably
X₂ is a small polar amino acid selected from the group consisting of S, T, D or N; even more preferably X₂ is T or S; particularly preferably X₂ is T; and
X₃ may be any amino acid; preferably X₃ is a small amino acid selected from the group consisting of G, A, S, C, N, D, T, V and P or a polar amino acid selected from the group consisting of N, K, R, Q, S, T, D, E, H, Y and W; more preferably
X₃ is a small polar amino acid selected from the group consisting of S, T, D or N; even more preferably X₃ is D or N; particularly preferably X₃ is D;
(v) a CDRL2 according to General Formula V:

EX₁X₂X₃RPS (V)

wherein
X₁ may be any amino acid; preferably X₁ is D, L, Q, A or G or a small amino acid selected from the group consisting of G, A, S, C, N, D, T, V and P; more preferably
X₁ is D or G;
X₂ may be any amino acid; preferably X₂ is a polar amino acid selected from the group consisting of N, K, R, Q, S, T, D, E, H, Y and W; more preferably X₂ is N, S or H; and
X₃ may be any amino acid; preferably X₃ is a polar amino acid selected from the group consisting of N, K, R, Q, S, T, D, E, H, Y and W; more preferably X₃ is K, R or Q; particularly preferably X₃ is K;
and
(vi) a CDRL3 according to General Formula VI:

X₁SYAX₂X₃STX₄X₅ (VI)

wherein
X₁ may be any amino acid; preferably X₁ is C, S, Q, or G or a small amino acid selected from the group consisting of G, A, S, C, N, D, T, V and P; more preferably X₁ is C;
X₂ may be any amino acid; preferably X₂ is a small amino acid selected from the group consisting of G, A, S, C, N, D, T, V and P or an aliphatic non-polar amino acid selected from the group consisting of A, G, I, L and V; more preferably X₂ is a small aliphatic non-polar amino acid selected from the group consisting of A, G and V; even more preferably X₂ is A or G;
X₃ may be any amino acid; preferably X₃ is I, S or T; more preferably X₃ is I or T;
X₄ may be any amino acid; preferably X₄ is a hydrophobic amino acid selected from the group consisting of A, T, P, C, V, R, F, Y, W, M, I and L; more preferably X₄ is L or W; even more preferably X₄ is L; and
X₅ may be any amino acid; preferably X₅ is an aliphatic non-polar amino acid selected from the group consisting of A, G, I, L and V; more preferably X₅ is I or V.

Accordingly, the present invention also provides an antibody, or an antigen-binding fragment thereof, comprising:
(i) a CDRH1 according to General Formula I:

X₁YYX₂H (I)

wherein
X₁ may be any amino acid; preferably X₁ is a small amino acid selected from the group consisting of G, A, S, C, N, D, T, V and P; more preferably X₁ is G or D; and
X₂ may be any amino acid; preferably X₂ is a nonpolar amino acid selected from the group consisting of A, C, G, I, L, M, F, P, W and V; more preferably X₂ is M or L; even more preferably X₂ is L;
(ii) a CDRH2 according to General Formula II:

RINPX₁X₂GX₃X₄X₅YAQX₆FQG (II)

wherein
X₁ may be any amino acid; preferably X₁ is N, K or G;
X₂ may be any amino acid; preferably X₂ is a small amino acid selected from the group consisting of G, A, S, C, N, D, T, V and P; more preferably X₂ is S or A; particularly preferably X₂ is S;
X₃ may be any amino acid; preferably X₃ is a small amino acid selected from the group consisting of G, A, S, C, N, D, T, V and P; more preferably X₃ is G or N;
X₄ may be any amino acid; preferably X₄ is a small amino acid selected from the group consisting of G, A, S, C, N, D, T, V and P, or a hydrophobic amino acid selected from the group consisting of A, T, P, C, V, R, F, Y, W, M, I and L; more preferably X₄ is a small hydrophobic amino acid selected from the group consisting of A, T, P, C, and V; even more preferably X₄ is T, V or A; particularly preferably X₄ is T;
X₅ may be any amino acid; preferably X₅ is a polar amino acid selected from the group consisting of N, K, R, Q, S, T, D, E, H, Y and W; more preferably X₅ is N, K, R or Q; particularly preferably X₅ is K or N; and
X₆ may be any amino acid; preferably X₆ is a polar amino acid selected from the group consisting of N, K, R, Q, S, T, D, E, H, Y and W; more preferably X₆ is K, R or Q; particularly preferably X₆ is K;
(iii) a CDRH3 according to SEQ ID NO: 5 or according to General Formula III:

VX₁IVVLRSTPTLYYX₂DY (III)

wherein
X₁ may be any amino acid; preferably X₁ is M, K or R; more preferably X₁ is K or R; even more preferably X₁ is K; and
X₂ may be any amino acid; preferably X₂ is a hydrophobic amino acid selected from the group consisting of A, T, P, C, V, R, F, Y, W, M, I and L; more preferably X₂ is F or L;
(iv) a CDRL1 according to General Formula IV:

TGX₁SSDVGX₂YX₃LVS (IV)

wherein
X₁ may be any amino acid; preferably X₁ is a small amino acid selected from the group consisting of G, A, S, C, N, D, T, V and P or a polar amino acid selected from the group consisting of N, K, R, Q, S, T, D, E, H, Y and W; more preferably
X₁ is a small polar amino acid selected from the group consisting of S, T, D or N; even more preferably X₁ is S or T;
X₂ may be any amino acid; preferably X₂ is a small amino acid selected from the group consisting of G, A, S, C, N, D, T, V and P or a polar amino acid selected from the group consisting of N, K, R, Q, S, T, D, E, H, Y and W; more preferably X₂ is a small polar amino acid selected from the group consisting of S, T, D or N; even more preferably X₂ is T or S; particularly preferably X₂ is T; and
X₃ may be any amino acid; preferably X₃ is a small amino acid selected from the group consisting of G, A, S, C, N, D, T, V and P or a polar amino acid selected from the group consisting of N, K, R, Q, S, T, D, E, H, Y and W; more preferably X₃ is a small polar amino acid selected from the group consisting of S, T, D or N; even more preferably X₃ is D or N; particularly preferably X₃ is D;
(v) a CDRL2 according to General Formula V:

EX₁X₂X₃RPS (V)

wherein
X₁ may be any amino acid; preferably X₁ is D, L, Q, A or G or a small amino acid selected from the group consisting of G, A, S, C, N, D, T, V and P; more preferably
X₁ is D or G;
X₂ may be any amino acid; preferably X₂ is a polar amino acid selected from the group consisting of N, K, R, Q, S, T, D, E, H, Y and W; more preferably X₂ is N, S or H; and
X₃ may be any amino acid; preferably X₃ is a polar amino acid selected from the group consisting of N, K, R, Q, S, T, D, E, H, Y and W; more preferably X₃ is K, R or Q; particularly preferably X₃ is K;
and
(vi) a CDRL3 according to General Formula VI:

X₁SYAX₂X₃STX₄X₅ (VI)

wherein
X₁ may be any amino acid; preferably X₁ is C, S, Q, or G or a small amino acid selected from the group consisting of G, A, S, C, N, D, T, V and P; more preferably X₁ is C;
X₂ may be any amino acid; preferably X₂ is a small amino acid selected from the group consisting of G, A, S, C, N, D, T, V and P or an aliphatic non-polar amino acid selected from the group consisting of A, G, I, L and V; more preferably X₂ is a small aliphatic non-polar amino acid selected from the group consisting of A, G and V; even more preferably X₂ is A or G;
X₃ may be any amino acid; preferably X₃ is I, S or T; more preferably X₃ is I or T;
X₄ may be any amino acid; preferably X₄ is a hydrophobic amino acid selected from the group consisting of A, T, P, C, V, R, F, Y, W, M, I and L; more preferably X₄ is L or W; even more preferably X₄ is L; and
X₅ may be any amino acid; preferably X₅ is an aliphatic non-polar amino acid selected from the group consisting of A, G, I, L and V; more preferably X₅ is I or V.

Such an antibody typically binds to a phosphorylated C-terminal domain or fragment of TDP-43 comprising amino acids 391-414 of SEQ ID NO: 1, wherein the serine residues at positions 403 and 404 of SEQ ID NO: 1 are phosphorylated, as described above. Moreover, such an antibody, or an antigen-binding fragment thereof, may exhibit further features (one or more thereof) as described above for the antibody binding (e.g., specifically binding) to a phosphorylated C-terminal domain or fragment of TDP-43 comprising amino acids 391-414 of SEQ ID NO: 1, wherein the serine residues at positions 403 and 404 of SEQ ID NO: 1 are phosphorylated.

Preferably, the antibody, or the antigen-binding fragment thereof, according to the present invention comprises:
(i) a CDRH1 according to General Formula I:

X₁YYX₂H (I)

wherein
X₁ is G or D; and
X₂ is L;
(ii) a CDRH2 according to General Formula II:

RINPX₁X₂GX₃X₄X₅YAQX₆FQG (II)

wherein
X₁ is N, K or G;
X₂ is S;
X₃ is G or N;
X₄ is T;
X₅ is K or N; and
X₆ is K;
(iii) a CDRH3 according to SEQ ID NO: 5 or according to General Formula III:

VX₁IVVLRSTPTLYYX₂DY (III)

wherein
X₁ is K; and
X₂ is F or L;
(iv) a CDRL1 according to General Formula IV:

TGX₁SSDVGX₂YX₃LVS (IV)

wherein
X₁ is S or T;
X₂ is T; and
X₃ is D;
(v) a CDRL2 according to General Formula V:

EX₁X₂X₃RPS (V)

wherein
X₁ is D or G;
X₂ is N, S or H; and
X₃ is K;
and
(vi) a CDRL3 according to General Formula VI:

X₁SYAX₂X₃STX₄X₅ (VI)

wherein
X₁ is C;
X₂ is A or G;
X₃ is I or T;
X₄ is L; and
X₅ is I or V.

In some embodiments, the antibody or the antigen-binding fragment comprises:
(i) a CDRH1 according to SEQ ID NO: 13;
(ii) a CDRH2 according to General Formula II:

RINPX₁X₂GX₃X₄X₅YAQX₆FQG (II)

wherein
X₁ is N or K;
X₂ is S;
X₃ is N;
X₄ is T;
X₅ is N; and
X₆ is K.
(iii) a CDRH3 according to General Formula III:

VX₁IVVLRSTPTLYYX₂DY (III)

wherein
X₁ is K; and
X₂ is F or L.
(iv) a CDRL1 according to General Formula IV:

TGX₁SSDVGX₂YX₃LVS (IV)

wherein
X₁ is S or T;
X₂ is T; and
X₃ is D;
(v) a CDRL2 according to General Formula V:

EX₁X₂X₃RPS (V)

wherein
X₁ is G;
X₂ is S or H; and
X₃ is K;
and
(vi) a CDRL3 according to SEQ ID NO: 17.

Exemplary amino acid sequences according to General Formula I include SEQ ID NOs 3 and 13. Exemplary amino acid sequences according to General Formula II include SEQ ID NOs 4, 14, 22 and 36-50. Exemplary amino acid sequences according to General Formula III include SEQ ID NOs 5, 15, 23, 51 and 52. Exemplary amino acid sequences according to General Formula IV include SEQ ID NOs 6 and 24. Exemplary amino acid sequences according to General Formula V include SEQ ID NOs 7, 16, 25, and 53-61. Exemplary amino acid sequences according to General Formula VI include SEQ ID NOs 8, 17 and 62-67.

In some embodiments, the antibody, or an antigen-binding fragment thereof, comprises (i) heavy chain CDR1, CDR2, and CDR3 sequences having at least 70% sequence identity with the amino acid sequences of SEQ ID NO: 3, SEQ ID NO: 4, and SEQ ID NO: 5, respectively, and light chain CDR1, CDR2, and CDR3 sequences having at least 70% sequence identity with the amino acid sequences of SEQ ID NO: 6, SEQ ID NO: 7, and SEQ ID NO: 8, respectively; or (ii) heavy chain CDR1, CDR2, and CDR3 sequences having at least 70% sequence identity with the amino acid sequences of SEQ ID NO: 13, SEQ ID NO: 14, and SEQ ID NO: 15, respectively, and light chain CDR1, CDR2, and CDR3 sequences having at least 70% sequence identity with the amino acid sequences of SEQ ID NO: 6, SEQ ID NO: 16, and SEQ ID NO: 17, respectively; or (iii) heavy chain CDR1, CDR2, and CDR3 sequences having at least 70% sequence identity with the amino acid sequences of SEQ ID NO: 13, SEQ ID NO: 22, and SEQ ID NO: 23, respectively, and light chain CDR1, CDR2, and CDR3 sequences having at least 70% sequence identity with the amino acid sequences of SEQ ID NO: 24, SEQ ID NO: 25, and SEQ ID NO: 17, respectively; or (iv) heavy chain CDR1, CDR2, and CDR3 sequences having at least 70% sequence identity with the amino acid sequences of SEQ ID NO: 28, SEQ ID NO: 29, and SEQ ID NO: 30, respectively, and light chain CDR1, CDR2, and CDR3 sequences having at least 70% sequence identity with the amino acid sequences of SEQ ID NO: 31, SEQ ID NO: 32, and SEQ ID NO: 33, respectively.

Accordingly, the present invention also provides an antibody, or an antigen-binding fragment thereof, comprising (i) heavy chain CDR1, CDR2, and CDR3 sequences having at least 70% sequence identity with the amino acid sequences of SEQ ID NO: 3, SEQ ID NO: 4, and SEQ ID NO: 5, respectively, and light chain CDR1, CDR2, and CDR3 sequences having at least 70% sequence identity with the amino acid sequences of SEQ ID NO: 6, SEQ ID NO: 7, and SEQ ID NO: 8, respectively; or (ii) heavy chain CDR1, CDR2, and CDR3 sequences having at least 70% sequence identity with the amino acid sequences of SEQ ID NO: 13, SEQ ID NO: 14, and SEQ ID NO: 15, respectively, and light chain CDR1, CDR2, and CDR3 sequences having at least 70% sequence identity with the amino acid sequences of SEQ ID NO: 6, SEQ ID NO: 16, and SEQ ID NO: 17, respectively; or (iii) heavy chain CDR1, CDR2, and CDR3 sequences having at least 70% sequence identity with the amino acid sequences of SEQ ID NO: 13, SEQ ID NO: 22, and SEQ ID NO: 23, respectively, and light chain CDR1, CDR2, and CDR3 sequences having at least 70% sequence identity with the amino acid sequences of SEQ ID NO: 24, SEQ ID NO: 25, and SEQ ID NO: 17, respectively; or (iv) heavy chain CDR1, CDR2, and CDR3 sequences having at least 70% sequence identity with the amino acid sequences of SEQ ID NO: 28, SEQ ID NO: 29, and SEQ ID NO: 30, respectively, and light chain CDR1, CDR2, and CDR3 sequences having at least 70% sequence identity with the amino acid sequences of SEQ ID NO: 31, SEQ ID NO: 32, and SEQ ID NO: 33, respectively.

Such an antibody typically binds to a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated, as described above. Moreover, such an antibody, or an antigen-binding fragment thereof, may exhibit further features (one or more thereof) as described above for the antibody binding (e.g., specifically binding) to a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated.

In some embodiments, the antibodies, or antigen-binding fragments thereof, as described herein comprise (i) heavy chain CDR1, CDR2, and CDR3 sequences having at least 80% sequence identity with the amino acid sequences of SEQ ID NO: 3, SEQ ID NO: 4, and SEQ ID NO: 5, respectively, and light chain CDR1, CDR2, and CDR3 sequences having at least 80% sequence identity with the amino acid sequences of SEQ ID NO: 6, SEQ ID NO: 7, and SEQ ID NO: 8, respectively; or (ii) heavy chain CDR1, CDR2, and CDR3 sequences having at least 80% sequence identity with the amino acid sequences of SEQ ID NO: 13, SEQ ID NO: 14, and SEQ ID NO: 15, respectively, and light chain CDR1, CDR2, and CDR3 sequences having at least 80% sequence identity with the amino acid sequences of SEQ ID NO: 6, SEQ ID NO: 16, and SEQ ID NO: 17, respectively; or (iii) heavy chain CDR1, CDR2, and CDR3 sequences having at least 80% sequence identity with the amino acid sequences of SEQ ID NO: 13, SEQ ID NO: 22, and SEQ ID NO: 23, respectively, and light chain CDR1, CDR2, and CDR3 sequences having at least 80% sequence identity with the amino acid sequences of SEQ ID NO: 24, SEQ ID NO: 25, and SEQ ID NO: 17, respectively; or (iv) heavy chain CDR1, CDR2, and CDR3 sequences having at least 80% sequence identity with the amino acid sequences of SEQ ID NO: 28, SEQ ID NO: 29, and SEQ ID NO: 30, respectively, and light chain CDR1, CDR2, and CDR3 sequences having at least 80% sequence identity with the amino acid sequences of SEQ ID NO: 31, SEQ ID NO: 32, and SEQ ID NO: 33, respectively.

The antibody, or an antigen-binding fragment thereof, of the present invention may comprise (i) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 3, SEQ ID NO: 4, and SEQ ID NO: 5, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 6, SEQ ID NO: 7, and SEQ ID NO: 8, respectively; or (ii) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 13, SEQ ID NO: 14, and SEQ ID NO: 15, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 6, SEQ ID NO: 16, and SEQ ID NO: 17, respectively; or (iii) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 13, SEQ ID NO: 22, and SEQ ID NO: 23, respectively, and light chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 24, SEQ ID NO: 25, and SEQ ID NO: 17, respectively; or (iv) heavy chain CDR1, CDR2, and CDR3 sequences having at least 90% sequence identity (e.g., 91%, 92%, 93%, 94%, 95%, 96%, 97%, 98%, 99% or more) with the amino acid sequences of SEQ ID NO: 28, SEQ ID NO: 29, and SEQ ID NO: 30, respectively, and light chain CDR1, CDR2, and CDR3 sequences having at least 90% sequence identity (e.g., 91%, 92%, 93%, 94%, 95%, 96%, 97%. 98%, 99% or more) with the amino acid sequences of SEQ ID NO: 31, SEQ ID NO: 32, and SEQ ID NO: 33, respectively.

As used throughout the present specification, “sequence identity” is usually calculated with regard to the full length of the reference sequence (i.e. the sequence recited in the application). Percentage identity, as referred to herein, can be determined, for example, by methods known in the art, such as BLAST using the default parameters specified by the NCBI (the National Center for Biotechnology Information; http://www.ncbi.nlm.nih.gov/) [Blosum 62 matrix; gap open penalty=11 and gap extension penalty=1].

For antibodies, percentage sequence identities can be determined when the subject and reference antibody sequences are maximally aligned using an appropriate alignment system for antibodies, e.g. IMGT or Kabat numbering convention (wherein the same system is applied to the distinct antibodies for comparison). After alignment, a subject antibody region (e.g., a specific CDR or the entire variable region of a heavy or light chain) can be compared with the corresponding region of the reference antibody. Thereby, gaps are typically not counted.

As used herein, a “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70% identical to the reference sequence. Variant sequences which are at least 70% identical have no more than 30 alterations, i.e. any combination of deletions, insertions or substitutions, per 100 amino acids of the reference sequence. In a “sequence variant” the functionality of the reference sequence (e.g., in the present case binding to the p(S403/404) C-terminal fragment or domain of TDP-43) may be maintained.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, cysteine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In general, in the antibody, or the antigen-binding fragment thereof, one or more, e.g., 1, 2, 3, 4, 5 or all 6 CDR sequences may carry one or more mutations, e.g. 1, 2, 3, 4, 5, 6 or more mutations. Preferred sequence alterations for the CDR-sequences in the antibodies, or antigen-binding fragments, of the present invention are those described above in General Formula (I)-(IV).

In some embodiments, the antibody, or an antigen-binding fragment thereof, of the present invention comprises:
(i) a CDH1 sequence according to SEQ ID NO: 3 or 13;
(ii) a CDRH2 sequence according to any one of SEQ ID NOs 4, 14, 22, 36-50;
(iii) a CDRH3 sequence according to any one of SEQ ID NOs 5, 15, 23, 51, and 52;
(iv) a CDRL1 sequence according to SEQ ID NO: 6 or 24;
(v) a CDRL2 sequence according to any one of SEQ ID NOs 7, 16, 25, 53-61; and
(vi) a CDRL3 sequence according to any one of SEQ ID NOs 8, 17, 62-67.

In some embodiments, the antibody, or an antigen-binding fragment thereof, may comprise heavy chain CDR1, CDR2, and CDR3 sequences according to SEQ ID NOs 3, 4 and 5, respectively, and light chain CDR1, CDR2, and CDR3 sequences according to SEQ ID NOs 6, 7 or 8, respectively. In some embodiments, the antibody, or an antigen-binding fragment thereof, may comprise heavy chain CDR1, CDR2, and CDR3 sequences according to SEQ ID NOs 3, 4 and 5, respectively, and light chain CDR1, CDR2, and CDR3 sequences according to SEQ ID NOs 6, 16 or 17, respectively. In some embodiments, the antibody, or an antigen-binding fragment thereof, may comprise heavy chain CDR1, CDR2, and CDR3 sequences according to SEQ ID NOs 3, 4 and 5, respectively, and light chain CDR1, CDR2, and CDR3 sequences according to SEQ ID NOs 24, 25 or 17, respectively. In some embodiments, the antibody, or an antigen-binding fragment thereof, may comprise heavy chain CDR1, CDR2, and CDR3 sequences according to SEQ ID NOs 3, 4 and 5, respectively, and light chain CDR1, CDR2, and CDR3 sequences according to SEQ ID NOs 31, 32 or 33, respectively.

In some embodiments, the antibody, or an antigen-binding fragment thereof, may comprise heavy chain CDR1, CDR2, and CDR3 sequences according to SEQ ID NOs 13, 14 and 15, respectively, and light chain CDR1, CDR2, and CDR3 sequences according to SEQ ID NOs 6, 7 or 8, respectively. In some embodiments, the antibody, or an antigen-binding fragment thereof, may comprise heavy chain CDR1, CDR2, and CDR3 sequences according to SEQ ID NOs 13, 14 and 15, respectively, and light chain CDR1, CDR2, and CDR3 sequences according to SEQ ID NOs 6, 16 or 17, respectively. In some embodiments, the antibody, or an antigen-binding fragment thereof, may comprise heavy chain CDR1, CDR2, and CDR3 sequences according to SEQ ID NOs 13, 14 and 15, respectively, and light chain CDR1, CDR2, and CDR3 sequences according to SEQ ID NOs 24, 25 or 17, respectively. In some embodiments, the antibody, or an antigen-binding fragment thereof, may comprise heavy chain CDR1, CDR2, and CDR3 sequences according to SEQ ID NOs 13, 14 and 15, respectively, and light chain CDR1, CDR2, and CDR3 sequences according to SEQ ID NOs 31, 32 or 33, respectively.

In some embodiments, the antibody, or an antigen-binding fragment thereof, may comprise heavy chain CDR1, CDR2, and CDR3 sequences according to SEQ ID NOs 13, 22 and 23, respectively, and light chain CDR1, CDR2, and CDR3 sequences according to SEQ ID NOs 6, 7 or 8, respectively. In some embodiments, the antibody, or an antigen-binding fragment thereof, may comprise heavy chain CDR1, CDR2, and CDR3 sequences according to SEQ ID NOs 13, 22 and 23, respectively, and light chain CDR1, CDR2, and CDR3 sequences according to SEQ ID NOs 6, 16 or 17, respectively. In some embodiments, the antibody, or an antigen-binding fragment thereof, may comprise heavy chain CDR1, CDR2, and CDR3 sequences according to SEQ ID NOs 13, 22 and 23, respectively, and light chain CDR1, CDR2, and CDR3 sequences according to SEQ ID NOs 24, 25 or 17, respectively. In some embodiments, the antibody, or an antigen-binding fragment thereof, may comprise heavy chain CDR1, CDR2, and CDR3 sequences according to SEQ ID NOs 13, 22 and 23, respectively, and light chain CDR1, CDR2, and CDR3 sequences according to SEQ ID NOs 31, 32 or 33, respectively.

In some embodiments, the antibody, or an antigen-binding fragment thereof, may comprise heavy chain CDR1, CDR2, and CDR3 sequences according to SEQ ID NOs 28, 29 and 30, respectively, and light chain CDR1, CDR2, and CDR3 sequences according to SEQ ID NOs 6, 7 or 8, respectively. In some embodiments, the antibody, or an antigen-binding fragment thereof, may comprise heavy chain CDR1, CDR2, and CDR3 sequences according to SEQ ID NOs 28, 29 and 30, respectively, and light chain CDR1, CDR2, and CDR3 sequences according to SEQ ID NOs 6, 16 or 17, respectively. In some embodiments, the antibody, or an antigen-binding fragment thereof, may comprise heavy chain CDR1, CDR2, and CDR3 sequences according to SEQ ID NOs 28, 29 and 30, respectively, and light chain CDR1, CDR2, and CDR3 sequences according to SEQ ID NOs 24, 25 or 17, respectively. In some embodiments, the antibody, or an antigen-binding fragment thereof, may comprise heavy chain CDR1, CDR2, and CDR3 sequences according to SEQ ID NOs 28, 29 and 30, respectively, and light chain CDR1, CDR2, and CDR3 sequences according to SEQ ID NOs 31, 32 or 33, respectively.

Preferably, the antibody or the antigen-binding fragment comprises:
(i) CDRH1, CDRH2, CDRH3, CDRL1, CDRL2 and CDRL3 sequences according to SEQ ID NOs 3-8, respectively;
(ii) CDRH1, CDRH2, CDRH3, CDRL1, CDRL2 and CDRL3 sequences according to SEQ ID NOs 13, 14, 15, 6, 16 and 17, respectively;
(iii) CDRH1, CDRH2, CDRH3, CDRL1, CDRL2 and CDRL3 sequences according to SEQ ID NOs 13, 22, 23, 24, 25 and 17, respectively; or
(iv) CDRH1, CDRH2, CDRH3, CDRL1, CDRL2 and CDRL3 sequences according to SEQ ID NOs 28-33, respectively.

In some embodiments, the CDRH1 of the antibody may comprise a sequence variant (e.g. having one or more (e.g. 1, 2, 3 or more) mutations compared to the antibody as occurring in nature). For example, the antibody may comprise a CDRH1 according to General Formula I, as described above, and, optionally, CDRH2, CDRH3, CDRL1, CDRL2 and CDRL3 sequences according to (i) SEQ ID NOs 4-8, respectively; (ii) SEQ ID NOs 14, 15, 6, 16 and 17, respectively; or (iii) SEQ ID NOs 22, 23, 24, 25 and 17, respectively. For example, the antibody may comprise a CDRH1 according to SEQ ID NO: 13 and, optionally, CDRH2, CDRH3, CDRL1, CDRL2 and CDRL3 sequences according to SEQ ID NOs 4-8, respectively. In some embodiments, the antibody may comprise a CDRH1 according to SEQ ID NO: 3 and, optionally, CDRH2, CDRH3, CDRL1, CDRL2 and CDRL3 sequences according to SEQ ID NOs 14, 15, 6, 16 and 17, respectively; or according to SEQ ID NOs 22, 23, 24, 25 and 17, respectively.

In some embodiments, the CDRH2 of the antibody may comprise a sequence variant (e.g. having one or more (e.g. 1, 2, 3, 4, 5, 6 or more) mutations compared to the antibody as occurring in nature). For example, the antibody may comprise a CDRH2 according to General Formula II, as described above, and, optionally, CDRH1, CDRH3, CDRL1, CDRL2 and CDRL3 sequences according to (i) SEQ ID NOs 3 and 5-8, respectively; (ii) SEQ ID NOs 13, 15, 6, 16 and 17, respectively; or (iii) SEQ ID NOs 13, 23, 24, 25 and 17, respectively. For example, the antibody may comprise a CDRH2 according to SEQ ID NO: 14 or 22 and, optionally, CDRH1, CDRH3, CDRL1, CDRL2 and CDRL3 sequences according to SEQ ID NOs 3 and 5-8, respectively. In other embodiments, the antibody may comprise a CDRH2 according to any one of SEQ ID NOs 42 to 46 and, optionally, CDRH1, CDRH3, CDRL1, CDRL2 and CDRL3 sequences according to SEQ ID NOs 3 and 5-8, respectively. In some embodiments, the antibody may comprise a CDRH2 according to SEQ ID NO: 4 or 22 and, optionally, CDRH1, CDRH3, CDRL1, CDRL2 and CDRL3 sequences according to SEQ ID NOs 13, 15, 6, 16 and 17, respectively. In other embodiments, the antibody may comprise a CDRH2 according to any one of SEQ ID NOs 36, 38, 39, 47 and 48 and, optionally, CDRH1, CDRH3, CDRL1, CDRL2 and CDRL3 sequences according to SEQ ID NOs 13, 15, 6, 16 and 17, respectively. In some embodiments, the antibody may comprise a CDRH2 according to SEQ ID NO: 4 or 14 and, optionally, CDRH1, CDRH3, CDRL1, CDRL2 and CDRL3 sequences according to SEQ ID NOs 13, 23, 24, 25 and 17, respectively. In other embodiments, the antibody may comprise a CDRH2 according to any one of SEQ ID NOs 37, 40, 41, 49 and 50 and, optionally, CDRH1, CDRH3, CDRL1, CDRL2 and CDRL3 sequences according to SEQ ID NOs 13, 23, 24, 25 and 17, respectively.

In some embodiments, the CDRH3 of the antibody may comprise a sequence variant (e.g. having one or more (e.g. 1, 2, 3, 4, 5, 6 or more) mutations compared to the antibody as occurring in nature). For example, the antibody may comprise a CDRH3 according to General Formula III, as described above, and, optionally, CDRH1, CDRH2, CDRL1, CDRL2 and CDRL3 sequences according to (i) SEQ ID NOs 3, 4, 6, 7 and 8, respectively; (ii) SEQ ID NOs 13, 14, 6, 16 and 17, respectively; or (iii) SEQ ID NOs 13, 22, 24, 25 and 17, respectively. In some embodiments, the antibody may comprise a CDRH3 according to SEQ ID NO: 5 or 23 and, optionally, CDRH1, CDRH2, CDRL1, CDRL2 and CDRL3 sequences according to SEQ ID NOs 13, 14, 6, 16 and 17, respectively. In other embodiments, the antibody may comprise a CDRH3 according to SEQ ID NO: 51 and, optionally, CDRH1, CDRH2, CDRL1, CDRL2 and CDRL3 sequences according to SEQ ID NOs 13, 14, 6, 16 and 17, respectively. In some embodiments, the antibody may comprise a CDRH3 according to SEQ ID NO: 5 or 15 and, optionally, CDRH1, CDRH2, CDRL1, CDRL2 and CDRL3 sequences according to SEQ ID NOs 13, 22, 24, 25 and 17, respectively. In other embodiments, the antibody may comprise a CDRH3 according to SEQ ID NO: 52 and, optionally, CDRH1, CDRH2, CDRL1, CDRL2 and CDRL3 sequences according to SEQ ID NOs 13, 22, 24, 25 and 17, respectively.

In some embodiments, the CDRL1 of the antibody may comprise a sequence variant (e.g. having one or more (e.g. 1, 2, 3, 4, 5, 6 or more) mutations compared to the antibody as occurring in nature). For example, the antibody may comprise a CDRL1 according to General Formula IV, as described above, and, optionally, CDRH1, CDRH2, CDRH3, CDRL2 and CDRL3 sequences according to (i) SEQ ID NOs 3, 4, 5, 7, and 8, respectively; (ii) SEQ ID NOs 13, 14, 15, 16 and 17, respectively; or (iii) SEQ ID NOs 13, 22, 23, 25 and 17, respectively. For example, the antibody may comprise a CDRL1 according to SEQ ID NO: 6 and, optionally, CDRH1, CDRH2, CDRH3, CDRL2 and CDRL3 sequences according to SEQ ID NOs 13, 22, 23, 25 and 17. In some embodiments, the antibody may comprise a CDRL1 according to SEQ ID NO: 24 and, optionally, CDRH1, CDRH2, CDRH3, CDRL2 and CDRL3 sequences according to SEQ ID NOs 3, 4, 5, 7, and 8, respectively; or according to SEQ ID NOs 13, 14, 15, 16 and 17, respectively.

In some embodiments, the CDRL2 of the antibody may comprise a sequence variant (e.g. having one or more (e.g. 1, 2, 3, 4 or more) mutations compared to the antibody as occurring in nature). For example, the antibody may comprise a CDRL2 according to General Formula V, as described above, and, optionally, CDRH1, CDRH2, CDRH3, CDRL1 and CDRL3 sequences according to (i) SEQ ID NOs 3, 4, 5, 6 and 8, respectively; (ii) SEQ ID NOs 13, 14, 15, 6 and 17, respectively; or (iii) SEQ ID NOs 13, 22, 23, 24 and 17, respectively. For example, the antibody may comprise a CDRL2 according to SEQ ID NO: 16 or 25 and, optionally CDRH1, CDRH2, CDRH3, CDRL1 and CDRL3 sequences according to SEQ ID NOs 3, 4, 5, 6 and 8, respectively. In other embodiments, the antibody may comprise a CDRL2 according to any one of SEQ ID NOs 53-57 and, optionally CDRH1, CDRH2, CDRH3, CDRL1 and CDRL3 sequences according to SEQ ID NOs 3, 4, 5, 6 and 8, respectively. In some embodiments, the antibody may comprise a CDRL2 according to SEQ ID NO: 7 or 25 and, optionally CDRH1, CDRH2, CDRH3, CDRL1 and CDRL3 sequences according to SEQ ID NOs 13, 14, 15, 6 and 17, respectively. In other embodiments, the antibody may comprise a CDRL2 according to SEQ ID NO: 58 or 59 and, optionally CDRH1, CDRH2, CDRH3, CDRL1 and CDRL3 sequences according to SEQ ID NOs 13, 14, 15, 6 and 17, respectively. In some embodiments, the antibody may comprise a CDRL2 according to SEQ ID NO: 16 or 25 and, optionally CDRH1, CDRH2, CDRH3, CDRL1 and CDRL3 sequences according to SEQ ID NOs 13, 22, 23, 24 and 17, respectively. In other embodiments, the antibody may comprise a CDRL2 according to SEQ ID NO: 60 or 61 and, optionally CDRH1, CDRH2, CDRH3, CDRL1 and CDRL3 sequences according to SEQ ID NOs 13, 22, 23, 24 and 17, respectively.

In some embodiments, the CDRL3 of the antibody may comprise a sequence variant (e.g. having one or more (e.g. 1, 2, 3, 4, 5, 6 or more) mutations compared to the antibody as occurring in nature). For example, the antibody may comprise a CDRL3 according to General Formula VI, as described above, and, optionally, CDRH1, CDRH2, CDRH3, CDRL1 and CDRL2 sequences according to (i) SEQ ID NOs 3-7, respectively; (ii) SEQ ID NOs 13, 14, 15, 6 and 16, respectively; or (iii) SEQ ID NOs 13, 22, 23, 24 and 25, respectively. For example, the antibody may comprise a CDRL3 according to SEQ ID NO: 17 and, optionally CDRH1, CDRH2, CDRH3, CDRL1 and CDRL2 sequences according to SEQ ID NOs 3-7, respectively. In other embodiments, the antibody may comprise a CDRL3 according to any one of SEQ ID NOs 62, 64 and 66 and, optionally CDRH1, CDRH2, CDRH3, CDRL1 and CDRL2 sequences according to SEQ ID NOs 3-7, respectively. In some embodiments, the antibody may comprise a CDRL3 according to SEQ ID NO: 8 and, optionally CDRH1, CDRH2, CDRH3, CDRL1 and CDRL2 sequences according to SEQ ID NOs 13, 14, 15, 6 and 16, respectively. In other embodiments, the antibody may comprise a CDRL3 according to any one of SEQ ID NOs 63, 65 and 67 and, optionally CDRH1, CDRH2, CDRH3, CDRL1 and CDRL2 sequences according to SEQ ID NOs 13, 14, 15, 6 and 16, respectively. In some embodiments, the antibody may comprise a CDRL3 according to SEQ ID NO: 8 and, optionally CDRH1, CDRH2, CDRH3, CDRL1 and CDRL2 sequences according to SEQ ID NOs 13, 22, 23, 24 and 25, respectively. In other embodiments, the antibody may comprise a CDRL3 according to any one of SEQ ID NOs 63, 65 and 67 and, optionally CDRH1, CDRH2, CDRH3, CDRL1 and CDRL2 sequences according to SEQ ID NOs 13, 22, 23, 24 and 25, respectively.

In some embodiments, the antibody, or the antigen-binding fragment thereof, comprises (i) a heavy chain variable region (VH) comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9 and a light chain variable region (VL) comprising the amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 10. Thereby, the CDR sequences as defined above (in particular heavy chain CDR1, CDR2, and CDR3 sequences as set forth in SEQ ID NO: 3, SEQ ID NO: 4, and SEQ ID NO: 5, respectively; and light chain CDR1, CDR2, and CDR3 sequences as set forth in SEQ ID NO: 6, SEQ ID NO: 7, and SEQ ID NO: 8, respectively; or the sequence variants thereof as described above) may be maintained.

In some embodiments, the antibody, or the antigen-binding fragment thereof, comprises (i) a heavy chain variable region (VH) comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 18 and a light chain variable region (VL) comprising the amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 19. Thereby, the CDR sequences as defined above (in particular heavy chain CDR1, CDR2, and CDR3 sequences as set forth in SEQ ID NO: 13, SEQ ID NO: 14, and SEQ ID NO: 15, respectively; and light chain CDR1, CDR2, and CDR3 sequences as set forth in SEQ ID NO: 6, SEQ ID NO: 16, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 26 and a light chain variable region (VL) comprising the amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 27. Thereby, the CDR sequences as defined above (in particular heavy chain CDR1, CDR2, and CDR3 sequences as set forth in SEQ ID NO: 13, SEQ ID NO: 22, and SEQ ID NO: 23, respectively; and light chain CDR1, CDR2, and CDR3 sequences as set forth in SEQ ID NO: 24, SEQ ID NO: 25, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 70% or more (e.g., 71%, 72%, 73%, 74%, 75%, 766%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 34 and a light chain variable region (VL) comprising the amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 35. Thereby, the CDR sequences as defined above (in particular heavy chain CDR1, CDR2, and CDR3 sequences as set forth in SEQ ID NO: 28, SEQ ID NO: 29, and SEQ ID NO: 30, respectively; and light chain CDR1, CDR2, and CDR3 sequences as set forth in SEQ ID NO: 31, SEQ ID NO: 32, and SEQ ID NO: 33, respectively; or the sequence variants thereof as described above) may be maintained.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 19. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 27. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 35.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 112. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 114.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 111 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 111 and a light chain variable region comprising an amino acid sequence having at least 70%, 75%, 80%, 85%, 90% or 95% identity to SEQ ID NO: 112. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 111 and a light chain variable region comprising an amino acid sequence having at least 70%, 75%, 80%, 85%, 90% or 95% identity to SEQ ID NO: 114.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 113 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 113 and a light chain variable region comprising an amino acid sequence having at least 70%, 75%, 80%, 85%, 90% or 95% identity to SEQ ID NO: 112. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 113 and a light chain variable region comprising an amino acid sequence having at least 70%, 75%, 80%, 85%, 90% or 95% identity to SEQ ID NO: 114.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 111 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 113 and a light chain variable region comprising an amino acid sequence having at least 70%, 75%, 80%, 85%, 90% or 95% identity to SEQ ID NO: 10.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 112. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 111 and a light chain variable region comprising an amino acid sequence having at least 70%, 75%, 80%, 85%, 90% or 95% identity to SEQ ID NO: 112. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 113 and a light chain variable region comprising an amino acid sequence having at least 70%, 75%, 80%, 85%, 90% or 95% identity to SEQ ID NO: 112.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 9 and a light chain variable region comprising an amino acid sequence having at least 70%, 75%, 80%, 85%, 90% or 95% identity to SEQ ID NO: 114. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 111 and a light chain variable region comprising an amino acid sequence having at least 70%, 75%, 80%, 85%, 90% or 95% identity to SEQ ID NO: 114. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 113 and a light chain variable region comprising an amino acid sequence having at least 70%, 75%, 80%, 85%, 90% or 95% identity to SEQ ID NO: 114.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 18 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 18 and a light chain variable region comprising an amino acid sequence having at least 70%, 75%, 80%, 85%, 90% or 95% identity to SEQ ID NO: 19. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 18 and a light chain variable region comprising an amino acid sequence having at least 70%, 75%, 80%, 85%, 90% or 95% identity to SEQ ID NO: 27. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 18 and a light chain variable region comprising an amino acid sequence having at least 70%, 75%, 80%, 85%, 90% or 95% identity to SEQ ID NO: 35.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 26 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 26 and a light chain variable region comprising an amino acid sequence having at least 70%, 75%, 80%, 85%, 90% or 95% identity to SEQ ID NO: 19. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 26 and a light chain variable region comprising an amino acid sequence having at least 70%, 75%, 80%, 85%, 90% or 95% identity to SEQ ID NO: 27. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 26 and a light chain variable region comprising an amino acid sequence having at least 70%, 75%, 80%, 85%, 90% or 95% identity to SEQ ID NO: 35.

In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 34 and a light chain variable region comprising an amino acid sequence having at least 70%, 75%, 80%, 85%, 90% or 95% identity to SEQ ID NO: 10. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 34 and a light chain variable region comprising an amino acid sequence having at least 70%, 75%, 80%, 85%, 90% or 95% identity to SEQ ID NO: 19. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 34 and a light chain variable region comprising an amino acid sequence having at least 70%, 75%, 80%, 85%, 90% or 95% identity to SEQ ID NO: 27. In some embodiments, the antibody, or an antigen-binding fragment thereof, may comprise a heavy chain variable region comprising an amino acid sequence having at least 70%, 75%, 80%, 85%, 90% or 95% identity to SEQ ID NO: 34 and a light chain variable region comprising an amino acid sequence having at least 70%, 75%, 80%, 85%, 90% or 95% identity to SEQ ID NO: 35.

Accordingly, the antibody, or the antigen-binding fragment thereof, preferably comprises (i) a heavy chain variable region comprising an amino acid sequence having at least 70% identity to SEQ ID NO: 9 and a light chain variable region comprising the amino acid sequence having at least 70% identity to SEQ ID NO: 10; or (ii) a heavy chain variable region comprising an amino acid sequence having at least 70% identity to SEQ ID NO: 18 and a light chain variable region comprising the amino acid sequence having at least 70% identity to SEQ ID NO: 19; or (iii) a heavy chain variable region comprising an amino acid sequence having at least 70% identity to SEQ ID NO: 26 and a light chain variable region comprising the amino acid sequence having at least 70% identity to SEQ ID NO: 27; or (iv) a heavy chain variable region comprising an amino acid sequence having at least 70% identity to SEQ ID NO: 34 and a light chain variable region comprising the amino acid sequence having at least 70% identity to SEQ ID NO: 35.

In some embodiments, the antibody, or the antigen-binding fragment thereof, comprises (i) a heavy chain variable region (VH) comprising an amino acid sequence having 75% or more identity to SEQ ID NO: 9 and a light chain variable region (VL) comprising the amino acid sequence having 75% or more identity to SEQ ID NO: 10. Thereby, the CDR sequences as defined above (in particular heavy chain CDR1, CDR2, and CDR3 sequences as set forth in SEQ ID NO: 3, SEQ ID NO: 4, and SEQ ID NO: 5, respectively; and light chain CDR1, CDR2, and CDR3 sequences as set forth in SEQ ID NO: 6, SEQ ID NO: 7, and SEQ ID NO: 8, respectively; or the sequence variants thereof as described above) may be maintained.

In some embodiments, the antibody, or the antigen-binding fragment thereof, comprises (i) a heavy chain variable region (VH) comprising an amino acid sequence having 75% or more identity to SEQ ID NO: 18 and a light chain variable region (VL) comprising the amino acid sequence having 75% or more identity to SEQ ID NO: 19. Thereby, the CDR sequences as defined above (in particular heavy chain CDR1, CDR2, and CDR3 sequences as set forth in SEQ ID NO: 13, SEQ ID NO: 14, and SEQ ID NO: 15, respectively; and light chain CDR1, CDR2, and CDR3 sequences as set forth in SEQ ID NO: 6, SEQ ID NO: 16, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 75% or more identity to SEQ ID NO: 26 and a light chain variable region (VL) comprising the amino acid sequence having 75% or more identity to SEQ ID NO: 27. Thereby, the CDR sequences as defined above (in particular heavy chain CDR1, CDR2, and CDR3 sequences as set forth in SEQ ID NO: 13, SEQ ID NO: 22, and SEQ ID NO: 23, respectively; and light chain CDR1, CDR2, and CDR3 sequences as set forth in SEQ ID NO: 24, SEQ ID NO: 25, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 75% or more identity to SEQ ID NO: 34 and a light chain variable region (VL) comprising the amino acid sequence having 75% or more identity to SEQ ID NO: 35. Thereby, the CDR sequences as defined above (in particular heavy chain CDR1, CDR2, and CDR3 sequences as set forth in SEQ ID NO: 28, SEQ ID NO: 29, and SEQ ID NO: 30, respectively; and light chain CDR1, CDR2, and CDR3 sequences as set forth in SEQ ID NO: 31, SEQ ID NO: 32, and SEQ ID NO: 33, respectively; or the sequence variants thereof as described above) may be maintained.

In some embodiments, the antibody, or the antigen-binding fragment thereof, comprises (i) a heavy chain variable region (VH) comprising an amino acid sequence having 80% or more identity to SEQ ID NO: 9 and a light chain variable region (VL) comprising the amino acid sequence having 80% or more identity to SEQ ID NO: 10. Thereby, the CDR sequences as defined above (in particular heavy chain CDR1, CDR2, and CDR3 sequences as set forth in SEQ ID NO: 3, SEQ ID NO: 4, and SEQ ID NO: 5, respectively; and light chain CDR1, CDR2, and CDR3 sequences as set forth in SEQ ID NO: 6, SEQ ID NO: 7, and SEQ ID NO: 8, respectively; or the sequence variants thereof as described above) may be maintained.

In some embodiments, the antibody, or the antigen-binding fragment thereof, comprises (i) a heavy chain variable region (VH) comprising an amino acid sequence having 80% or more identity to SEQ ID NO: 18 and a light chain variable region (VL) comprising the amino acid sequence having 80% or more identity to SEQ ID NO: 19. Thereby, the CDR sequences as defined above (in particular heavy chain CDR1, CDR2, and CDR3 sequences as set forth in SEQ ID NO: 13, SEQ ID NO: 14, and SEQ ID NO: 15, respectively; and light chain CDR1, CDR2, and CDR3 sequences as set forth in SEQ ID NO: 6, SEQ ID NO: 16, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 80% or more identity to SEQ ID NO: 26 and a light chain variable region (VL) comprising the amino acid sequence having 80% or more identity to SEQ ID NO: 27. Thereby, the CDR sequences as defined above (in particular heavy chain CDR1, CDR2, and CDR3 sequences as set forth in SEQ ID NO: 13, SEQ ID NO: 22, and SEQ ID NO: 23, respectively; and light chain CDR1, CDR2, and CDR3 sequences as set forth in SEQ ID NO: 24, SEQ ID NO: 25, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 80% or more identity to SEQ ID NO: 34 and a light chain variable region (VL) comprising the amino acid sequence having 80% or more identity to SEQ ID NO: 35. Thereby, the CDR sequences as defined above (in particular heavy chain CDR1, CDR2, and CDR3 sequences as set forth in SEQ ID NO: 28, SEQ ID NO: 29, and SEQ ID NO: 30, respectively; and light chain CDR1, CDR2, and CDR3 sequences as set forth in SEQ ID NO: 31, SEQ ID NO: 32, and SEQ ID NO: 33, respectively; or the sequence variants thereof as described above) may be maintained.

In some embodiments, the antibody, or the antigen-binding fragment thereof, comprises (i) a heavy chain variable region (VH) comprising an amino acid sequence having 85% or more identity to SEQ ID NO: 9 and a light chain variable region (VL) comprising the amino acid sequence having 85% or more identity to SEQ ID NO: 10. Thereby, the CDR sequences as defined above (in particular heavy chain CDR1, CDR2, and CDR3 sequences as set forth in SEQ ID NO: 3, SEQ ID NO: 4, and SEQ ID NO: 5, respectively; and light chain CDR1, CDR2, and CDR3 sequences as set forth in SEQ ID NO: 6, SEQ ID NO: 7, and SEQ ID NO: 8, respectively; or the sequence variants thereof as described above) may be maintained.

In some embodiments, the antibody, or the antigen-binding fragment thereof, comprises (i) a heavy chain variable region (VH) comprising an amino acid sequence having 85% or more identity to SEQ ID NO: 18 and a light chain variable region (VL) comprising the amino acid sequence having 85% or more identity to SEQ ID NO: 19. Thereby, the CDR sequences as defined above (in particular heavy chain CDR1, CDR2, and CDR3 sequences as set forth in SEQ ID NO: 13, SEQ ID NO: 14, and SEQ ID NO: 15, respectively; and light chain CDR1, CDR2, and CDR3 sequences as set forth in SEQ ID NO: 6, SEQ ID NO: 16, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 85% or more identity to SEQ ID NO: 26 and a light chain variable region (VL) comprising the amino acid sequence having 85% or more identity to SEQ ID NO: 27. Thereby, the CDR sequences as defined above (in particular heavy chain CDR1, CDR2, and CDR3 sequences as set forth in SEQ ID NO: 13, SEQ ID NO: 22, and SEQ ID NO: 23, respectively; and light chain CDR1, CDR2, and CDR3 sequences as set forth in SEQ ID NO: 24, SEQ ID NO: 25, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 85% or more identity to SEQ ID NO: 34 and a light chain variable region (VL) comprising the amino acid sequence having 85% or more identity to SEQ ID NO: 35. Thereby, the CDR sequences as defined above (in particular heavy chain CDR1, CDR2, and CDR3 sequences as set forth in SEQ ID NO: 28, SEQ ID NO: 29, and SEQ ID NO: 30, respectively; and light chain CDR1, CDR2, and CDR3 sequences as set forth in SEQ ID NO: 31, SEQ ID NO: 32, and SEQ ID NO: 33, respectively; or the sequence variants thereof as described above) may be maintained.

In some embodiments, the antibody, or the antigen-binding fragment thereof, comprises (i) a heavy chain variable region (VH) comprising an amino acid sequence having 90% or more identity to SEQ ID NO: 9 and a light chain variable region (VL) comprising the amino acid sequence having 90% or more identity to SEQ ID NO: 10. Thereby, the CDR sequences as defined above (in particular heavy chain CDR1, CDR2, and CDR3 sequences as set forth in SEQ ID NO: 3, SEQ ID NO: 4, and SEQ ID NO: 5, respectively; and light chain CDR1, CDR2, and CDR3 sequences as set forth in SEQ ID NO: 6, SEQ ID NO: 7, and SEQ ID NO: 8, respectively; or the sequence variants thereof as described above) may be maintained.

In some embodiments, the antibody, or the antigen-binding fragment thereof, comprises (i) a heavy chain variable region (VH) comprising an amino acid sequence having 90% or more identity to SEQ ID NO: 18 and a light chain variable region (VL) comprising the amino acid sequence having 90% or more identity to SEQ ID NO: 19. Thereby, the CDR sequences as defined above (in particular heavy chain CDR1, CDR2, and CDR3 sequences as set forth in SEQ ID NO: 13, SEQ ID NO: 14, and SEQ ID NO: 15, respectively; and light chain CDR1, CDR2, and CDR3 sequences as set forth in SEQ ID NO: 6, SEQ ID NO: 16, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 90% or more identity to SEQ ID NO: 26 and a light chain variable region (VL) comprising the amino acid sequence having 90% or more identity to SEQ ID NO: 27. Thereby, the CDR sequences as defined above (in particular heavy chain CDR1, CDR2, and CDR3 sequences as set forth in SEQ ID NO: 13, SEQ ID NO: 22, and SEQ ID NO: 23, respectively; and light chain CDR1, CDR2, and CDR3 sequences as set forth in SEQ ID NO: 24, SEQ ID NO: 25, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 90% or more identity to SEQ ID NO: 34 and a light chain variable region (VL) comprising the amino acid sequence having 90% or more identity to SEQ ID NO: 35. Thereby, the CDR sequences as defined above (in particular heavy chain CDR1, CDR2, and CDR3 sequences as set forth in SEQ ID NO: 28, SEQ ID NO: 29, and SEQ ID NO: 30, respectively; and light chain CDR1, CDR2, and CDR3 sequences as set forth in SEQ ID NO: 31, SEQ ID NO: 32, and SEQ ID NO: 33, respectively; or the sequence variants thereof as described above) may be maintained.

In some embodiments, the antibody, or the antigen-binding fragment thereof, comprises (i) a heavy chain variable region (VH) comprising an amino acid sequence having 95% or more identity to SEQ ID NO: 9 and a light chain variable region (VL) comprising the amino acid sequence having 95% or more identity to SEQ ID NO: 10. Thereby, the CDR sequences as defined above (in particular heavy chain CDR1, CDR2, and CDR3 sequences as set forth in SEQ ID NO: 3, SEQ ID NO: 4, and SEQ ID NO: 5, respectively; and light chain CDR1, CDR2, and CDR3 sequences as set forth in SEQ ID NO: 6, SEQ ID NO: 7, and SEQ ID NO: 8, respectively; or the sequence variants thereof as described above) may be maintained.

In some embodiments, the antibody, or the antigen-binding fragment thereof, comprises (i) a heavy chain variable region (VH) comprising an amino acid sequence having 95% or more identity to SEQ ID NO: 18 and a light chain variable region (VL) comprising the amino acid sequence having 95% or more identity to SEQ ID NO: 19. Thereby, the CDR sequences as defined above (in particular heavy chain CDR1, CDR2, and CDR3 sequences as set forth in SEQ ID NO: 13, SEQ ID NO: 14, and SEQ ID NO: 15, respectively; and light chain CDR1, CDR2, and CDR3 sequences as set forth in SEQ ID NO: 6, SEQ ID NO: 16, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 95% or more identity to SEQ ID NO: 26 and a light chain variable region (VL) comprising the amino acid sequence having 95% or more identity to SEQ ID NO: 27. Thereby, the CDR sequences as defined above (in particular heavy chain CDR1, CDR2, and CDR3 sequences as set forth in SEQ ID NO: 13, SEQ ID NO: 22, and SEQ ID NO: 23, respectively; and light chain CDR1, CDR2, and CDR3 sequences as set forth in SEQ ID NO: 24, SEQ ID NO: 25, and SEQ ID NO: 17, respectively; or the sequence variants thereof as described above) may be maintained.

In some embodiments, the antibody, or the antigen-binding fragment thereof, comprises (i) a heavy chain variable region (VH) comprising an amino acid sequence having 95% or more identity to SEQ ID NO: 34 and a light chain variable region (VL) comprising the amino acid sequence having 95% or more identity to SEQ ID NO: 35. Thereby, the CDR sequences as defined above (in particular heavy chain CDR1, CDR2, and CDR3 sequences as set forth in SEQ ID NO: 28, SEQ ID NO: 29, and SEQ ID NO: 30, respectively; and light chain CDR1, CDR2, and CDR3 sequences as set forth in SEQ ID NO: 31, SEQ ID NO: 32, and SEQ ID NO: 33, respectively; or the sequence variants thereof as described above) may be maintained.

In some embodiments, the antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 10. The antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 19. The antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 27. The antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 35. The antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 112. The antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 114.

In some embodiments, the antibody, or an antigen-binding fragment thereof, may comprise a heavy chain variable region comprising an amino acid sequence as set forth in SEQ ID NO: 111 and a light chain variable region comprising an amino acid sequence as set forth in SEQ ID NO: 10. The antibody, or an antigen-binding fragment thereof, may comprise a heavy chain variable region comprising an amino acid sequence as set forth in SEQ ID NO: 111 and a light chain variable region comprising an amino acid sequence as set forth in SEQ ID NO: 19. The antibody, or an antigen-binding fragment thereof, may comprise a heavy chain variable region comprising an amino acid sequence as set forth in SEQ ID NO: 111 and a light chain variable region comprising an amino acid sequence as set forth in SEQ ID NO: 27. The antibody, or an antigen-binding fragment thereof, may comprise a heavy chain variable region comprising an amino acid sequence as set forth in SEQ ID NO: 111 and a light chain variable region comprising an amino acid sequence as set forth in SEQ ID NO: 35. The antibody, or an antigen-binding fragment thereof, may comprise a heavy chain variable region comprising an amino acid sequence as set forth in SEQ ID NO: 111 and a light chain variable region comprising an amino acid sequence as set forth in SEQ ID NO: 112. The antibody, or an antigen-binding fragment thereof, may comprise a heavy chain variable region comprising an amino acid sequence as set forth in SEQ ID NO: 111 and a light chain variable region comprising an amino acid sequence as set forth in SEQ ID NO: 114.

In some embodiments, the antibody, or an antigen-binding fragment thereof, may comprise a heavy chain variable region comprising an amino acid sequence as set forth in SEQ ID NO: 113 and a light chain variable region comprising an amino acid sequence as set forth in SEQ ID NO: 10. The antibody, or an antigen-binding fragment thereof, may comprise a heavy chain variable region comprising an amino acid sequence as set forth in SEQ ID NO: 113 and a light chain variable region comprising an amino acid sequence as set forth in SEQ ID NO: 19. The antibody, or an antigen-binding fragment thereof, may comprise a heavy chain variable region comprising an amino acid sequence as set forth in SEQ ID NO: 113 and a light chain variable region comprising an amino acid sequence as set forth in SEQ ID NO: 27. The antibody, or an antigen-binding fragment thereof, may comprise a heavy chain variable region comprising an amino acid sequence as set forth in SEQ ID NO: 113 and a light chain variable region comprising an amino acid sequence as set forth in SEQ ID NO: 35. The antibody, or an antigen-binding fragment thereof, may comprise a heavy chain variable region comprising an amino acid sequence as set forth in SEQ ID NO: 113 and a light chain variable region comprising an amino acid sequence as set forth in SEQ ID NO: 112. The antibody, or an antigen-binding fragment thereof, may comprise a heavy chain variable region comprising an amino acid sequence as set forth in SEQ ID NO: 113 and a light chain variable region comprising an amino acid sequence as set forth in SEQ ID NO: 114.

In some embodiments, the antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 10. The antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 19. The antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 27. The antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 35. The antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 112. The antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 114.

In some embodiments, the antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 10. The antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 19. The antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 27. The antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 35. The antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 112. The antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 114.

In some embodiments, the antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 10. The antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 19. The antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 27. The antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 35. The antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 112. The antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 114.

Preferably, the antibody, or an antigen-binding fragment thereof, may comprise a heavy chain variable region comprising an amino acid sequence as set forth in SEQ ID NO: 9 and a light chain variable region comprising an amino acid sequence as set forth in SEQ ID NO: 10.

It is also preferred that the antibody, or an antigen-binding fragment thereof, may comprise a heavy chain variable region comprising an amino acid sequence as set forth in SEQ ID NO: 18 and a light chain variable region comprising an amino acid sequence as set forth in SEQ ID NO: 19.

It is also preferred that the antibody, or an antigen-binding fragment thereof, may comprise a heavy chain variable region comprising an amino acid sequence as set forth in SEQ ID NO: 26 and a light chain variable region comprising an amino acid sequence as set forth in SEQ ID NO: 27.

It is also preferred that the antibody, or an antigen-binding fragment thereof, may comprise a heavy chain variable region comprising an amino acid sequence as set forth in SEQ ID NO: 34 and a light chain variable region comprising an amino acid sequence as set forth in SEQ ID NO: 35.

The CDR and VH/VL sequences of exemplified antibodies of the invention, namely antibodies 31F3, 30E3 (also referred to herein as “30E1”), 9F11 and 15D7 are shown in Table 1 below. Table 1 also includes exemplified variant antibodies of 31F3.

TABLE 1
CDR and VH/VL sequences (SEQ ID NOs) of
exemplified antibodies of the invention.

| | CDRH1 | CDRH2 | CDRH3 | CDRL1 | CDRL2 | CDRL3 | VH | VL |
|---|---|---|---|---|---|---|---|---|
| 31F3 | 3 | 4 | 5 | 6 | 7 | 8 | 9 | 10 |
| 31F3 HfLf | 3 | 4 | 5 | 6 | 7 | 8 | 111 | 112 |
| 31F3HfLg | 3 | 4 | 5 | 6 | 7 | 8 | 111 | 114 |
| 31F3HgLf | 3 | 4 | 5 | 6 | 7 | 8 | 113 | 112 |
| 31F3HgLg | 3 | 4 | 5 | 6 | 7 | 8 | 113 | 114 |
| 30E1 | 13 | 14 | 15 | 6 | 16 | 17 | 18 | 19 |
| 9F11 | 13 | 22 | 23 | 24 | 25 | 17 | 26 | 27 |
| 15D7 | 28 | 29 | 30 | 31 | 32 | 33 | 34 | 35 |

Antibodies, and antigen-binding fragments thereof, comprising the CDR sequences (in particular the sets of CDRH1, CDRH2, CDRH3, CDRL1, CDRL2 and CDRL3) and/or the VH/VL sequence pairs are particularly preferred. Antibodies, and antigen-binding fragments thereof, comprising sequence variants thereof, in particular as described above (e.g., according to General Formulas (I)-(IV)), are also included in the scope of the present invention.

In some embodiments, the antibody, or the antigen-binding fragment thereof, comprises the (complete) set of six CDRs of any one of the exemplified antibodies shown in Table 1. Preferably, the antibody also comprises the respective VH/VL sequences having at least 70%, at least 75%, at least 80%, at least 85%, at least 90%, at least 95% sequence identity, wherein the CDR sequences are preferably maintained (i.e. any mutations compared to the reference sequence occurs in the framework regions). In some embodiments, the VH and/or VL sequences may be those as shown in Table 1; or they may differ in 0, 1, 2, 3, 4, 5, 6, 7, 8, 9, 10 or more mutations.

In some embodiments, the VH sequences of Table 1, e.g. SEQ ID NO: 9, may include up to six mutations. For example, the amino acids at any one or more of the positions 5, 13, 69, 83, 84 and 88 of SEQ ID NO: 9 may be substituted, e.g. by a conservative substitution. For example, a variant of SEQ ID NO: 9 may include any one of the following substitutions: E5V, R13K, S69T, V83L, N84V and/or P88S.

In some embodiments, the VL sequences of Table 1, e.g. SEQ ID NO: 10, may include up to four mutations. For example, the amino acids at any one or more of the positions 49, 60, 83 and 103 of SEQ ID NO: 10 may be substituted, e.g. by a conservative substitution. For example, a variant of SEQ ID NO: 10 may include any one of the following substitutions: 149M, L60V, D83E and/or G103S.

Preferably, the antibody of the invention is a human antibody. In some embodiments, the antibody of the invention is a monoclonal antibody. For example, the antibody of the invention may be a human monoclonal antibody.

The exemplary antibodies of the invention (as shown in Table 1) were identified in humans. Accordingly, those antibodies are fully human antibodies. Human antibodies are advantageous as compared to antibodies of non-human origin, because non-human antibodies, including chimeric and humanized antibodies, can trigger an adverse immune response, which can lead to nausea, diarrhea and flu-like symptoms. In more severe cases, these side-effects can even be lethal. Non-human antibody segments often trigger immune responses in humans (anti-drug antibodies (ADA)), thereby not only eliciting undesired side effects, but also reducing the efficacy of the non-human antibody in humans. In contrast thereto, antibodies retrieved from humans have a higher safety profile, as the antibodies have proven tolerability in the human body, which is combined with the outstanding affinity maturation typical of the human immune system. As used herein, the term “human antibodies” not only includes antibodies originally found in humans, but also sequence variants thereof, wherein specific amino acid residues (but not entire antibody segments) are mutated. In contrast to non-human and humanized antibodies, which usually contain entire antibody segments (such as entire sets of CDR sequences) of non-human origin, sequence variants of human antibodies typically contain only selective/specific mutations within select antibody segments (e.g., within a CDR or framework region and/or within a constant region; e.g. to modify the antibodies' affinity, functionality, half-life, etc.).

For example, a human antibody according to the present invention may comprise only a limited number of mutations per CDR (e.g. no more than 6, preferably no more than 5, more preferably no more than 4, even more preferably no more than 3, still more preferably no more than 2 and particularly preferably only a single mutation per CDR), as compared to the sequences shown in Table 1. In case of more than two mutations they may not occur in a consecutive manner (to avoid creating a non-human sequence segment). The same applies to the framework regions (or the entire VH/VL sequences) as well to the constant regions. The latter may carry, for example, specific modifications known in the art to modify the antibody's (Fc-related) functionality, as described herein below.

Antibodies of the invention can be of any isotype (e.g., IgA, IgG, IgM i.e. an α, γ or μ heavy chain). For example, the antibody may be of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a lambda or kappa light chain.

In some embodiments, the antibody according to the present invention, or an antigen binding fragment thereof, comprises an Fc moiety. The Fc moiety may be derived from human origin, e.g. from human IgG1, IgG2, IgG3, and/or IgG4, such as human IgG1 or IgG4.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

Each of the amino acid positions within an Fc moiety have been numbered herein according to the art-recognized EU numbering system of Kabat, see e.g., by Kabat et al., in “Sequences of Proteins of Immunological Interest”, U.S. Dept. Health and Human Services, 1983 and 1987. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E. A., National Institutes of Health (U.S.) Office of the Director, “Sequences of Proteins of Immunological Interest”, 5^{th} edition, Bethesda, MD: U.S. Dept. of Health and Human Services, Public Health Service, National Institutes of Health, 1991, hereby entirely incorporated by reference).

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprise one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art.

In some embodiments, the antibody according to the present invention comprises a (complete) Fc moiety/Fc region, wherein the interaction/binding with FcR is not compromised. In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren PWHI, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of GM-CSF by autoantibodies in pulmonary alveolar proteinosis. Nat Commun 2015, 6:1-9).

The antibody according to the present invention can be modified by introducing amino acid mutations, e.g. into the CH2 or CH3 domain of the heavy chain, in order to alter the binding affinity for FcR and/or their serum half-life in comparison to unmodified antibodies. Examples of such modifications include, but are not limited to, the Fc modifications as described in Saxena A, Wu D. Advances in Therapeutic Fc Engineering—Modulation of IgG-Associated Effector Functions and Serum Half-life. Front Immunol. 2016; 7:580, which is incorporated herein by reference. For example, the antibody may comprise the “YTE” mutations (M252Y/S254T/T256E; EU numbering). In some embodiments, the antibody may comprise the mutations M428L and/or N434S in the heavy chain constant region (EU numbering).

In general, FcR binding can be mediated by the interaction of the Fc moiety (of an antibody) with Fc receptors (FcRs), which are specialized cell surface receptors on hematopoietic cells. Fc receptors belong to the immunoglobulin superfamily, and were shown to mediate both the removal of antibody-coated pathogens by phagocytosis of immune complexes, and the lysis of erythrocytes and various other cellular targets coated with the corresponding antibody, via antibody dependent cell mediated cytotoxicity (ADCC).

In some embodiments, the Fc moiety of the antibody may comprise or consist of at least the portion of an Fc moiety that is known in the art to be required for FcRn binding or extended half-life. Alternatively or additionally, the Fc moiety of the antibody comprises at least the portion of known in the art to be required for Protein A binding and/or the Fc moiety of the antibody of the invention comprises at least the portion of an Fc molecule known in the art to be required for protein G binding. The Fc moiety may comprise at least the portion known in the art to be required for FcγR binding. Accordingly, an Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

Regarding FcγRI binding, modification in native IgG of at least one of E233-G236, P238, D265, N297, A327 and P329 is known to decrease binding to FcγRI. IgG2 residues at positions 233-236, substituted into IgG1 and IgG4, decreases binding to FcγRI by 10³-fold and eliminated the human monocyte response to antibody-sensitized red blood cells (Armour, K. L., et al. Eur. J. Immunol. 29 (1999) 2613-2624).

Regarding binding to FcγRII, two regions of native IgG Fc appear to be critical for interactions of FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Decreased binding for FcγRIIA is found e.g. for IgG mutation of at least one of E233-G236, P238, D265, N297, A327, P329, D270, Q295, A327, R292 and K414. Mapping of the binding sites on human IgG1 for Fc receptors, the above-mentioned mutation sites and methods for measuring binding to FcγRI and FcγRIIA are described in Shields, R. L., et al., J. Biol. Chem. 276 (2001) 6591-6604. Moreover, antibodies, and antigen binding fragments thereof, of the invention may be able to bind to FcγRIIb. Antibodies, or antigen binding fragments thereof, of the invention may comprise an engineered Fc moiety with the mutations S267E and L328F, in particular as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933. Thereby, the clearance of immune complexes can be enhanced.

Regarding FcγRIII binding, decreased binding to FcγRIIIA is found e.g. for mutation of at least one of E233-G236, P238, D265, N297, A327, P329, D270, Q295, A327, S239, E269, E293, Y296, V303, A327, K338 and D376. For example, a single (S239D or I332E), double (S239D/I332E), and triple mutations (S239D/I332E/A330L) improved the affinity for human FcγRIIIa. Furthermore, the addition of the mutation G236A to S239D/I332E improved not only the FcγRIIa:FcγRIIb ratio, but also enhanced binding to FcγRIIIa. Accordingly, the mutations G236A/S239D/A330L/I332E may be introduced, e.g. to enhance engagement of FcγRIIa and FcγRilla.

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. While the Fc region often contains Fc moieties of distinct antibody (heavy) chains, in some embodiments, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

In some embodiments, the Fc moiety, or the Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

As outlined above, an antibody according to the present invention may comprise a (complete) Fc region derived from human IgG1 or IgG4. In some embodiments, the antibody according to the present invention comprises, in particular in addition to a (complete) Fc region derived from human IgG1 or IgG4 also all other parts of the constant regions of IgG, such as all other parts of the constant regions of (human) IgG1 or IgG4. In some embodiments, the antibody according to the present invention comprises, one or more constant region(s), in particular constant region(s) of IgG, such as (a) constant region(s) of (human) IgG1 or IgG4. The antibody according to the present invention may comprise, for example, all constant regions of IgG (such as (human) IgG1 or IgG4).

Example sequences of constant regions are the amino acid sequences according to SEQ ID NOs: 68-70. For example, the amino acid sequence of IgG1 CH1-CH2-CH3 is according to SEQ ID NO: 68 or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity.

Example sequences of entire heavy and light chains (including the variable and constant regions) include the amino acid sequences according to SEQ ID NO: 11 and 12 for the heavy and light chain, respectively, of antibody 31F3 and the amino acid sequences according to SEQ ID NO: 20 and 21 for the heavy and light chain, respectively, of antibody 30E3 (also referred to herein as “30E1”). For example, the antibody may comprise a heavy chain comprising an amino acid sequence according to SEQ ID NO: 11 or 20, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity; and/or a light chain comprising an amino acid sequence according to SEQ ID NO: 12 or 21, or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity.

Antibodies of the invention also include hybrid antibody molecules that comprise the six CDRs from an antibody as disclosed herein and one or more CDRs from another antibody to an antigen. For example, the antibody may be bispecific. A bispecific (or multispecific) antibody, or antigen-binding fragment thereof, according to the present invention may comprise at least one specificity (antigen-binding site of an antibody) as described herein. In some embodiments, the bispecific (or multispecific) antibody, or antigen-binding fragment thereof, binds to two distinct epitopes of (pathological) TDP-43.

Variant antibodies are also included within the scope of the invention. Thus, variants of the sequences recited in the application are also included within the scope of the invention. Such variants include natural variants generated by somatic mutation in vivo during the immune response or in vitro upon culture of B cell clones. Variants may also arise due to the degeneracy of the genetic code or may be produced due to errors in transcription or translation. Moreover, variants may be produced by targeted introduction of mutation(s), e.g. using site-directed mutagenesis, as known in the art.

Antibodies of the invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

In general, the antibody may be of any antibody format known in the art, including antigen-binding fragments (which retain the antigen-binding activity of the antibodies). To this end, the antigen-binding fragments may comprise the CDRs from an antibody of the invention. Antigen-binding fragments include, but are not limited to, single chain antibodies, Fab, Fab′, F(ab′)2 or Fv. Also included are heavy or light chain monomers and dimers, single domain heavy chain antibodies, single domain light chain antibodies as well as V_{HH}/V_{H}.

The invention encompasses, in particular, single chain antibodies, such as single-chain Fv fragments (scFv), which are typically derived from the heavy and light chains of an antibody of the invention, e.g. in which the heavy and light chain variable domains are joined by a peptide linker. As peptide linker, for example the 15 amino acid linker GGGGS (SEQ ID NO: 71) (3) or the 20 amino acid linker GGGGS(4) may be used between the heavy (VH) and light (VL) chain variable regions. Fv fragments, such as scFv, may not comprise constant regions, but only the variable regions. Thereby, a soluble and flexible amino acid peptide linker may be used to connect the V regions to a scFv (single chain fragment variable) fragment for stabilization of the molecule. Further examples of antibody formats include, but are not limited to, disulfide-bond stabilized scFv (ds-scFv), single chain Fab (scFab), scFv-zipper, scFv-Fc, di- and multimeric antibody formats like dia-, tria- and tetra-bodies, and minibodies (miniAbs; scFv-CH3) that comprise different formats consisting of scFvs linked to oligomerization domains.

Furthermore, also V_{HH}/V_{H} of camelid heavy chain antibodies and single domain antibodies (sdAb) are included in the scope of the invention. A single-domain antibody (sdAb), also known as a nanobody, is an antibody fragment comprising only a single monomeric variable antibody domain. Single domain antibodies may be polypeptides comprising one variable domain (VH) of a heavy-chain antibody, or of a common IgG. They may be of about 110 amino acids in length. They may be obtained by splitting the dimeric variable domains from common immunoglobulin G (IgG) from humans into monomers. Although mostly heavy chain variable domains are used in this approach, also nanobodies derived from light chain variable domains were shown to bind specifically to target epitopes.

Antibodies and antibody fragments of the invention may be contained in a variety of structures known to the person skilled in the art. In the context of the present invention, intrabodies may be useful, e.g. to target cytoplasmic TDP-43 aggregation. Accordingly, the antibody (or the antigen-binding fragment thereof) may be an intrabody.

As used herein, an “intrabody” is an intracellular antibody (or antigen-binding fragment thereof), in particular an antibody (or antigen-binding fragment thereof) that has been designed to be expressed (and act) intracellularly. Typically, intrabodies target intracellular antigens; in the present case for example intracellular, such as cytoplasmic, TDP-43. To obtain intrabodies, they may be delivered into and/or expressed in the target cell. This may be achieved, e.g., by using/administering (recombinant) nucleic acid molecules encoding the intrabody. For delivery, nucleic acids may be administered directly or by using a vector, such as viral delivery vectors known in the art (e.g., adenoviral or adeno-associated viral vectors) or non-viral delivery systems (e.g., nanoparticles, protein transduction domain peptides or modified liposomes).

In general, intrabodies may be of any antibody format. Preferably single-chain antibody formats are used, such as scFv or a single-domain antibody. Intrabody approaches are well-established for single-chain antibody formats, which do not require assembly from two polypeptide chains like classical IgG antibodies, thereby eliminating the need for bicistronic vectors and the associated problems to achieve the correct heavy chain/light chain ratio upon expression.

While naturally occurring antibodies are optimized to be secreted from the cell, an intrabody can be directed to a specific target antigen inside a cell, in particular to a specific subcellular locations including the cytosol, nucleus, endoplasmic reticulum (ER), mitochondria, peroxisomes, plasma membrane and trans-Golgi network (TGN). To this end, in-frame fusion with intracellular trafficking/localization peptide sequences may be used. Classical antibodies typically contain a leader sequence at the N terminus for secretion of the immunoglobulin, which may be modified for targeting of subcellular localizations. For example, ER-retained intrabodies may be designed with a leader sequence at the N-terminus and a retention peptide, KDEL (SEQ ID NO: 72), at the C-terminus. Protein retention in the trans-Golgi can be achieved with a trans-Golgi retention signal (e.g., as described in Zhou P, Goldstein S, Devadas K, Tewari D, Notkins A L (1998) Cells transfected with a non-neutralizing antibody gene are resistant to HIV infection: targeting the endoplasmic reticulum and trans-Golgi network. J Immunol 160:1489-96). In order to target the plasma membrane a receptor transmembrane domain may be used (e.g., as described in Chesnut J D, Baytan A R, Russell M, Chang M P, Bernard A, Maxwell I H, Hoeffler J P (1996) Selective isolation of transiently transfected cells from a mammalian cell population with vectors expressing a membrane anchored single-chain antibody. J Immunol Methods 193:17-27). To allow cytoplasmic expression, the leader sequence of variable heavy (VH) and variable light (VL) domains, which target antibody fragments to the lumen of the endoplasmic reticulum, may be removed (leader-less). Nuclear targeting can be achieved by adding one or more nuclear localization sequences (NLS) to the leader-less antibody fragments, such as the PKKKRKV (SEQ ID NO: 73) sequence of the large T antigen of SV40, either at the N- and C-terminus. Preferably, however, the intrabody is not localized in the nucleus. To this end, the sequence of the antibody/intrabody may contain a nuclear export signal (NES). Nuclear export signals are known in the art. Non-limiting examples of nuclear export signals can be found in the database NESdb, as described in Xu et al. NESdb: a database of NES-containing CRM1 cargoes; Molecular Biology of the Cell 2012 23:18, pp. 3673-3676. Nuclear export signals are usually small peptides (e.g., 8-15 amino acids) with regularly spaced hydrophobic residues.

In particular for cytoplasmic location (e.g., to target cytoplasmic TDP-43 aggregates), the antibody may be modified to improve its stability and structure for cytoplasmic expression.

Examples of modifications for cytoplasmic expression include, but are not limited to, modifications of immunoglobulin VL domains for hyperstability, selection of antibodies resistant to the more reducing cytosolic environment, expression as a fusion protein with maltose binding protein or other stable intracellular proteins (e.g., as described in Shelly Shaki-Loewenstein, Rahely Zfania, Stephen Hyland, Winfried S. Wels, Itai Benhar, A universal strategy for stable intracellular antibodies, Journal of Immunological Methods, Volume 303, Issues 1-2, 2005, pages 19-39) and the use of scaffolds that are known to be particularly suitable for folding correctly in the cytosol and onto which the antigen binding features of other antibodies are grafted (e.g. as described in Donini M., Morea V., Desiderio A., Pashkoulov D., Villani M. E., Tramontano A., et al. Engineering stable cytoplasmic intrabodies with designed specificity, J Mol Biol, 330 (2) (2003), pp. 323-332). Preferred immunoglobulin frameworks with enhanced stability and methods of obtaining them are disclosed, for example, in WO 03/097697 A2 and in Wörn A, Auf der Maur A, Escher D, Honegger A, Barberis A, Plückthun A. Correlation between in vitro stability and in vivo performance of anti-GCN4 intrabodies as cytoplasmic inhibitors. J Biol Chem. 2000 Jan. 28; 275(4):2795-803. doi: 10.1074/jbc.275.4.2795, which are incorporated herein by reference.

More recently, a method for engineering an ultra-stable cytoplasmic antibody has been established, which uses peptide tags with a highly negative charge and a low isoelectric point. This method (also referred to as the “STAND” method) is described in Kabayama et al., 2020 (Kabayama, H., Takeuchi, M., Tokushige, N. et al. An ultra-stable cytoplasmic antibody engineered for in vivo applications. Nat Commun 11, 336 (2020). https://doi.org/10.1038/s41 467-019-13654-9; which is incorporated herein by reference) and may be used to obtain stable cytoplasmic intrabodies. Accordingly, the antibody (in particular an scFv) of the present invention may include an s3Flag (DYKDHDGDYKDHDI-DYKDDDDK; SEQ ID NO: 74) and/or a human influenza hemagglutinin (HA) peptide tag (YPYDVPDYA; SEQ ID NO: 75). For example, the antibody may be a polypeptide comprising (in N- to C-terminal direction) s3Flag-scFv-HA.

Nucleic Acids

In another aspect, the invention also provides a nucleic acid molecule comprising a polynucleotide encoding the antibody according to the present invention, or an antigen-binding fragment thereof, as described above.

In some embodiments, the nucleic acid molecule comprises one or more polynucleotide(s) encoding the exemplified antibodies of the invention (e.g., as described above, in particular in Table 1), or a sequence variant thereof as described herein (e.g., having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity as described above).

Exemplified nucleic acid sequences encoding the CDR and VH/VL sequences of exemplified antibodies of the invention are shown in Table 2 below.

TABLE 2
Exemplified nucleic acid CDR and VH/VL sequences (SEQ
ID NOs) of exemplified antibodies of the invention.

| | CDRH1 | CDRH2 | CDRH3 | CDRL1 | CDRL2 | CDRL3 | VH | VL |
|---|---|---|---|---|---|---|---|---|
| 31F3 | 76 | 77 | 78 | 79 | 80 | 81 | 82 | 83 |
| 30E1 | 86 | 87 | 88 | 89 | 90 | 91 | 92 | 93 |
| 9F11 | 86 | 96 | 97 | 98 | 99 | 91 | 100 | 101 |
| 15D7 | 102 | 103 | 104 | 105 | 106 | 107 | 108 | 109 |

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody (or a single chain antibody). In other words, the light chain and the heavy chain of the antibody may be encoded by the same nucleic acid molecule (e.g., for single chain antibodies or for antibodies with separate heavy and light chains in bicistronic manner or an expression cassette containing more than one ribosome entry site such as IRES). Alternatively, the light chain and the heavy chain of the antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, heterologous UTR (e.g., for optimal translation/expression), a heterologous poly-A-tail, heterologous DNA insulator elements and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33 (Web Server issue):W526-31; or, for example, Genscript's OptimumGene™ algorithm (as described in US 2011/0081708 A1).

For example, the nucleic acid molecule of the invention may comprise a nucleic acid sequence as set forth in any one of SEQ ID NOs 76-109; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

In some embodiments, the encoded antibody, or an antigen-binding fragment thereof, may be an intrabody. As described above, intrabodies may be expressed in the target cell and, therefore, nucleic acid molecules encoding the intrabody may be delivered to the target cell (e.g., by administration to a patient either directly or using a viral or non-viral delivery vector).

The present invention also provides a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof. The above description regarding the (general) features of the nucleic acid molecule of the invention applies accordingly to the first and second nucleic acid molecule of the combination. Accordingly, one or both of the polynucleotides encoding the heavy and/or light chain(s) of the antibody, or an antigen-binding fragment thereof, may be codon-optimized. For example, the combination may comprise a nucleic acid sequence as set forth in any one of SEQ ID NOs 76-109; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

Vectors

Further included within the scope of the invention are vectors, for example, expression vectors, comprising a nucleic acid molecule according to the present invention. Usually, a vector comprises a nucleic acid molecule as described above.

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multiple cloning site, a heterologous promotor, a heterologous enhancer, a heterologous selection marker (to identify cells comprising said vector in comparison to cells not comprising said vector), heterologous origin of replications, heterologous DNA insulator elements and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

For the expression of intrabodies, specific expression vectors may be used, which facilitate the expression of scFv fragments as secreted or intracellular proteins, such as the integrated system of scFvexpress vectors (as described in: Persic L, Righi M, Roberts A, Hoogenboom H R, Cattaneo A, Bradbury A (1997) Targeting vectors for intracellular immunization. Gene 187:1-8). The scFvexpress plasmids may contain an N- and/or C-terminal localization signal for targeting of the antibody to different cell compartments, such as the endoplasmic reticulum (scFvex-ER), the cytoplasm (scFvex-cyt), the nucleus (scFvex-nuc) and the mitochondria (scFvex-mit). Accordingly, a specific expression vector may be selected according to the desired intracellular localization of the intrabody.

As used herein, the term “vector” may also refer to a delivery vector, e.g. for viral or non-viral delivery of a nucleic acid of the invention. Alternatively, it may be referred to viral or non-viral delivery systems. Accordingly, the present invention also provides a delivery vector/system comprising the nucleic acid molecule as described above (or comprising an expression vector as described above). The delivery vector/system may be viral or non-viral. Various examples of viral and non-viral delivery vectors/systems are known in the art and described, for example, in Nayerossadat N, Maedeh T, Ali P A. Viral and nonviral delivery systems for gene delivery. Adv Biomed Res. 2012; 1:27. doi:10.4103/2277-9175.98152, which is incorporated herein by reference. Non-limiting examples of viral delivery vectors/systems include retroviral vectors; adenoviral vectors; adeno-associated viral (AAV) vectors, including helper-dependent adenoviral vectors and hybrid adenoviral vectors; herpes simplex virus vectors; lentivirus vectors; poxvirus vectors and Epstein-Barr virus vectors. Among the viral vectors, adenoviral vectors and adeno-associated viral (AAV) vectors are preferred. Non-limiting examples of non-viral delivery vectors/systems include chemical and non-chemical methods. Non-chemical delivery includes physical methods, such as electroporation and other methods for transient penetration of the cell membrane by mechanical, electrical, ultrasonic, hydrodynamic, or laser-based energy; naked DNA or RNA delivery; gene gun; hydrodynamic delivery; ultrasound delivery and magnetofection. Chemical non-viral delivery systems include cationic particles, in particular cationic lipids/liposomes, cationic polymers and lipid/polymer systems. Among non-viral vectors/systems, cationic liposomes are preferred.

Cells

In a further aspect, the present invention also provides a (host) cell expressing the antibody according to the present invention, or an antigen-binding fragment thereof; and/or comprising the vector (or the combination of vectors) according the present invention. The (host) cell may be an isolated cell, which is not part of a human or animal body, e.g. a cell line or an engineered cell. The cell may express the nucleic acid(s) or vector(s) of the invention in a recombinant manner, e.g. in a heterologous manner (i.e., the cell/cell type does not express the antibody or the antigen-binding fragment in nature).

Examples of such cells include, but are not limited to, eukaryotic cells, e.g., yeast cells, animal cells or plant cells. Other examples of such cells include, but are not limited to, prokaryotic cells, e.g. E. coli. In some embodiments, the cells are mammalian cells, such as a mammalian cell line. Examples include human cells, CHO cells, HEK293 cells, PER.C6 cells, NS0 cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., decreased immunogenicity). Accordingly, the antibody of the invention, or the antigen-binding fragment thereof, may have a post-translational modification, which is distinct from the naturally produced antibody (e.g., an antibody of an immune response in a human).

Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983).

Standard techniques of molecular biology may be used to prepare DNA sequences encoding the antibodies or antigen-binding fragments of the present invention. Desired DNA sequences may be synthesized completely or in part, e.g., using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293, PER.C6, NS0, myeloma or hybridoma cells. Also, prokaryotic, e.g. bacterial host cell expression systems may be used for the production of antibody molecules, such as complete antibody molecules. Suitable bacterial host cells include, but are not limited to, E. coli cells.

Accordingly, the present invention provides a method for preparing the antibody, or an antigen-binding fragment or an immunoglobulin chain(s) thereof, according to the present invention, said method comprising
(i) culturing the host cell as described above; and
(ii) isolating the antibody or immunoglobulin chain(s) thereof from the culture.

In other words, the present invention also provides a process for the production of an antibody molecule according to the present invention comprising culturing a (heterologous) host cell comprising a vector encoding a nucleic acid of the present invention, in particular under conditions suitable for expression of protein from DNA encoding the antibody molecule of the present invention, and isolating the antibody molecule.

For production of the antibody comprising both heavy and light chains, a host cell, such as a cell line, may be transfected with two vectors, a first vector encoding a light chain polypeptide and a second vector encoding a heavy chain polypeptide, e.g. as described above. Alternatively, a single vector may be used, the vector including sequences encoding light chain and heavy chain polypeptides (e.g. for single chain antibodies or in a bicistronic manner).

Thus, the invention also provides a method for preparing a recombinant cell, comprising the steps of: (i) providing one or more nucleic acids that encode(s) the antibody of the invention; (ii) inserting the nucleic acid into an expression vector and (iii) transfecting the vector into a (heterologous) host cell in order to permit expression of the antibody of interest in that host cell. The nucleic acid of step (i) may, but need not, be manipulated to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

The transfected host cell may be a eukaryotic cell, including yeast and animal cells, particularly mammalian cells (e.g., CHO cells, NS0 cells, human cells such as PER.C6, HEK293 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest. The transfected host cell population may be prepared by (i) providing nucleic acid(s) encoding a selected antibody of interest, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest.

In some embodiments, antibodies according to the invention may be produced by (i) expressing a nucleic acid sequence according to the invention in a host cell, e.g. by use of a vector (or host cell) according to the present invention, and (ii) isolating the expressed antibody product. Additionally, the method may include (iii) purifying the isolated antibody.

Accordingly, after production, the antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Fusion Proteins and Immunoconjugates

In a further aspect, the present invention also provides a fusion protein or an immunoconjugate comprising the antibody, or an antigen-binding fragment thereof, according to the present invention as described above.

In general, the antibody, or an antigen-binding fragment thereof, may be fused to a variety of moieties, in particular to add or increase certain functionalities of the antibody, as known in the art. For example, antibodies may be fused to markers to facilitate their detection, such as GFP. Other fusions, in particular with tags, may be useful for purification, such as a His tag. Accordingly, the present invention also provides a fusion protein comprising (i) the antibody according to the present invention and (ii) a distinct peptide or a protein, such as label, e.g. a fluorescent peptide or protein, e.g. EGFP. Further examples of fusion proteins include fusion proteins, which lead to degradation of the target, such as ubiquitin ligases and other proteasome/autophagy targeting/degradation inducing enzymes/tags, for example as described in Gao H, Sun X, Rao Y. PROTAC Technology: Opportunities and Challenges. ACS Med Chem Lett. 2020 Mar. 12; 11(3):237-240. doi: 10.1021/acsmedchemlett.9b00597; or in Chassin, H., Müller, M., Tigges, M. et al. A modular degron library for synthetic circuits in mammalian cells. Nat Commun 10, 2013 (2019). https://doi.org/10.1038/s41467-019-09974-5; which are incorporated herein by reference.

As used herein, the term “immunoconjugate” refers to the antibody, or antigen binding fragment thereof, conjugated to a further moiety. Antibodies of the invention can be conjugated to a variety of moieties, e.g., to improve the therapeutic/diagnostic properties of the antibody, to facilitate detection, or for imaging or treatment purposes. For example, antibodies of the invention may be conjugated to therapeutic agents, prodrugs, peptides, proteins, enzymes, viruses, lipids, biological response modifiers, pharmaceutical agents, PEG, a detectable label and/or transport moieties.

Antibody conjugates (i.e. antibodies conjugated to other molecules) are known in the art. In particular, the molecule conjugated to the antibody may be linked to the antibody by a cleavable or non-cleavable linker (e.g., as described in: Thomas H. Pillow. Novel linkers and connections for antibody-drug conjugates to treat cancer and infectious disease. Pharmaceutical Patent Analyst Vol. 6, No. 1, Feb. 3, 2017, https://doi.org/10.4155/ppa-2016-0032; or in: Beck A, Goetsch L, Dumontet C, Corvaïa N. Strategies and challenges for the next generation of antibody-drug conjugates. Nat Rev Drug Discov. 2017 May; 16(5):315-337). Examples of such linkers, which may be used to link the molecule to the antibody or antigen binding fragment, are described, for example in EP 2927227 and in Thomas H. Pillow. Novel linkers and connections for antibody-drug conjugates to treat cancer and infectious disease. Pharmaceutical Patent Analyst Vol. 6, No. 1, Feb. 3, 2017, https://doi.org/10.4155/ppa-2016-0032. Further examples of domains for conjugation include genetically modified cross-reacting material (CRM) of diphtheria toxin, tetanus toxoid (T), meningococcal outer membrane protein complex (OMPC), diphtheria toxoid (D), and H. influenzae protein D (HiD), for example as described in Pichichero M E: Protein carriers of conjugate vaccines: characteristics, development, and clinical trials, Hum Vaccin Immunother. 2013 December; 9(12):2505-23. Optionally, linkers may be used between the antibodies of the invention and the molecules to be conjugated to the antibodies (such as a label), e.g., as described in U.S. Pat. No. 4,831,175. In some embodiments, antibodies or, antigen-binding fragments thereof may be directly labeled with radioactive iodine, indium, yttrium, or other radioactive particle known in the art, e.g., as described in U.S. Pat. No. 5,595,721.

For example, antibodies of the invention, or the antigen binding fragments thereof, may be coupled to a detectable label, for example to provide measurability, e.g. for quantification or to facilitate imaging. Labeled antibodies may be employed in a wide variety of assays, in particular in immunoassays, employing a wide variety of labels. Preferred labels include radionuclides, enzymes, coenzymes, fluorescers, chemiluminescers, chromogens, enzyme substrates or co-factors, enzyme inhibitors, prosthetic group complexes, free radicals, particles, dyes (e.g., fluorescent dyes, tandem dyes), and the like. Examples of suitable enzymes include horseradish peroxidase (HRP), alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or ³H. Such labeled reagents may be used in a variety of well-known assays, such as radioimmunoassays, enzyme immunoassays, e.g., ELISA, fluorescent immunoassays, and the like, preferably in ELISA. Labeled antibodies according to the present invention may be thus be used in such assays for example as described in U.S. Pat. Nos. 3,766,162; 3,791,932; 3,817,837; and 4,233,402.

Preferred labels include (i) enzymes as described above, e.g. horseradish peroxidase (HRP) or alkaline phosphatase, in particular in Blockade-of-binding assay, Western Blotting, ELISA and immunohistochemistry; (ii) prosthetic group complexes, e.g. streptavidin/biotin and avidin/biotin, in particular in ELISA and immunohistochemistry; (iii) fluorescers as described above, such as fluorescent dyes and fluorescent proteins (e.g., (enhanced) green fluorescent protein (EGFP); mTagBFP, mTurquoise, mVenus, mKO2, mCherry, mApple, mKate2), in particular in immunofluorescence and flow cytometry; and (iv) tandem dyes in flow cytometry.

Accordingly, the present invention also provides an immunoconjugate comprising the antibody, or an antigen-binding fragment thereof, according to the present invention as described above and a detectable label and/or a transport moiety.

As used herein, the term “detectable label” refers to moieties such as peptide sequences, fluorescent proteins or other molecules, which are capable of producing, either directly or indirectly, a detectable signal and which can be appended or introduced into the antibody as described herein. Examples of detectable labels include various enzymes, prosthetic groups, fluorescent materials, luminescent materials, bioluminescent materials, chemiluminescent (chromophore) compounds, radioactive materials, positron emitting metals for use in positron emission tomography, and nonradioactive paramagnetic metal ions. Moreover, a second antibody, or antibody fragment thereof, may also be used as label. In this case, the antibody, or the antigen binding fragment thereof, according to the present invention is conjugated to a second antibody, or antibody fragment thereof, to form an antibody heteroconjugate, e.g. as described in U.S. Pat. No. 4,676,980. In this case, the second antibody may optionally be labelled as described herein. In some embodiments, the detectable label may be detectable indirectly, for example using secondary antibody.

Methods for coupling antibodies to labels are well known in the art. For example, in the antibody or in the antigen binding fragment thereof the side chain of lysine, which terminates in a primary amine (—NH₂), may be used to link labels covalently to the antibody or in the antigen binding fragment thereof. Many variant labeling procedures are described in the literature. For example, the labelling approach may be selected from the group consisting of NHS esters, heterobifunctional reagents, carbodiimides and sodium periodate.

In some embodiments, the label may be radio-opaque, positron-emitting radionuclide (for example for use in PET imaging), or a radioisotope, such as ³H, ¹³N, ¹⁴C, ¹⁸F, ³²P, ³⁵S, ⁹⁹Tc, ¹¹¹In, ¹²³I, ¹²⁵I, and ¹³¹I. Further examples of radioisotopes are described in Schubert M, Bergmann R, Förster C, Sihver W, Vonhoff S, Klussmann S, Bethge L, Walther M, Schlesinger J, Pietzsch J, Steinbach J, Pietzsch H J; Novel Tumor Pretargeting System Based on Complementary I-Configured Oligonucleotides; Bioconjug Chem. 2017 Apr. 19; 28(4):1176-1188 and in Bhusari P, Vatsa R, Singh G, Parmar M, Bal A, Dhawan D K, Mittal B R, Shukla J; Development of Lu-177-trastuzumab for radioimmunotherapy of HER2 expressing breast cancer and its feasibility assessment in breast cancer patients; Int J Cancer. 2017 Feb. 15; 140(4):938-947. Preferred examples of radioisotopes include ⁹⁰Y, ¹³¹I, and ¹⁷⁷Lu.

In some embodiments, the label may be a metal ion. Examples of metal ions useful to be conjugated to antibodies, e.g. for use as diagnostics, can be found, e.g., in U.S. Pat. No. 4,741,900.

In some embodiments, the label may be a fluorescent (fluorophore) or chemiluminescent (chromophore) compound. Examples of suitable fluorescent materials include umbelliferone, fluorescein, fluorescein isothiocyanate, rhodamine, dichlorotriazinyiamine fluorescein, dansyl chloride or phycoerythrin; examples of bioluminescent materials include luciferase, luciferin and aequorin. In some embodiments, the antibody may be detectably labeled by coupling it to a chemiluminescent compound. The presence of the chemiluminescent-tagged antibody may then be determined by detecting the presence of luminescence that arises during the course of a chemical reaction. Examples of particularly useful chemiluminescent labeling compounds are luminol, isoluminol, theromatic acridmium ester, imidazole, aeridinium salt and oxalate ester.

Further examples of labels, which may be conjugated to the antibody or antigen binding fragment of the present invention, include quantum dots, and iron oxide. An example of iron oxide nanoparticles is described in Hengyi Xu, Zoraida P. Aguilar, Lily Yang, Min Kuang, Hongwei Duan, Yonghua Xiong, Hua Wei, and Andrew Wang: Antibody Conjugated Magnetic Iron Oxide Nanoparticles for Cancer Cell Separation in Fresh Whole Blood. Biomaterials. 2011 December; 32(36): 9758-9765.

In some embodiments, the label may be a prosthetic group complex, e.g. streptavidin/biotin or avidin/'biotin. In some embodiments, the antibody, or the antigen binding fragment thereof, according to the present invention may be biotinylated. Biotinylation is rapid, specific and is unlikely to perturb the natural function of the molecule due to the small size of biotin (MW=244.31 g/mol). Biotin binds to streptavidin and avidin with an extremely high affinity, fast on-rate, and high specificity. Biotin-binding to streptavidin and avidin is resistant to extremes of heat, pH and proteolysis, making capture of biotinylated molecules possible in a wide variety of environments. The antibody, or the antigen binding fragment thereof, according to the present invention may be biotinylated chemically or enzymatically. Chemical biotinylation utilizes various conjugation chemistries to yield nonspecific biotinylation of amines (e.g., NHS-coupling gives biotinylation of any primary amines in the antibody, see below). Enzymatic biotinylation results in biotinylation of a specific lysine within a certain sequence by use of a bacterial biotin ligase.

In some embodiments, the antibody, or the antigen-binding fragment may be conjugated to an enzyme. The immunoconjugate comprising the antibody and the enzyme may be used, for example, in an enzyme immunoassay (EIA) (Voller, A, “The Enzyme Linked Immunosorbent Assay (ELISA)” Microbiological Associates Quarterly Publication, Walkersville, Md., Diagnostic Horizons 2 (1978), 1-7; Voller et al, J. Clin. Pathol. 31 (1978), 507-520; Butler, Meth. Enzymol. 73 (1981), 482-523; Maggio, E. (ed.). Enzyme Immunoassay, CRC Press, Boca Raton, Fla., (1980); Ishikawa, E. et al, (eds.), Enzyme Immunoassay, Kgaku Shoin, Tokyo (1981)). Thereby, the enzyme conjugated to the antibody may react with an appropriate substrate, such as a chromogenic substrate. Thereby, a chemical moiety which can be detected, for example, by spectrophotometric, fluorimetric or by visual means, may be produced. In some embodiments, the detection can be accomplished by colorimetric methods which employ a chromogenic substrate for the enzyme. Detection can also be accomplished by visual comparison of the extent of enzymatic reaction of a substrate in comparison with similarly prepared standards.

Examples of suitable enzymes include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, giucose-6-phosphate dehydrogenase, glucoamylase and acetylcholinesterase. In some embodiments, the enzyme is horseradish peroxidase (HRP). Conjugation of antibodies to HRP are, for example, described in Wisdom G B. Conjugation of antibodies to horseradish peroxidase. Methods Mol Biol. 2005; 295:127-30 or in Antibodies—a laboratory manual. Edited by Edward A. Greenfield, Second edition 2012, Cold Spring Harbor Laboratory Press, ISBN: 9781936113811.

Alternatively or in addition to the label as described above, the antibody, or the antigen-binding fragment thereof, may be conjugated to a transport moiety.

A transport moiety typically mediates the transport of the antibody to a target, e.g. a specific part in the (human) body or a cell. Preferred transport moieties facilitate the transport across the blood brain barrier and/or into a cell.

In some embodiments, the antibody may be conjugated to a transport moiety that facilitates entry into a cell, e.g. for treatment of a TDP-43 proteinopathy or for diagnostic detection of intracellular aggregated TDP43. For example, the antibody can be chemically linked or recombinantly fused to a cell-penetrating peptide such as trans-activating transcriptional activator (TAT) and TAT derivatives, penetratin or transportan.

In some embodiments, the antibody may be conjugated to an agent facilitating the passage across the blood-brain barrier (BBB). Agents facilitating the passage across the blood-brain barrier include, but are not limited to, agents undergoing adsorptive mediated transport (AMT) and agents undergoing receptor-mediated transcytosis (RMT).

Non-limiting examples of agents undergoing adsorptive mediated transport (AMT) include sugar molecules (for example for glycation), diamines and polyamines (for example for polyamination), and cell penetrating peptides. Examples of diamines and polyamines include hexamethylenediamine, putrescine, spermidine and spermine. Examples of cell penetrating peptides include penetratin (derived from Antennapedia protein), TAT protein (HIV-1 trans-activating transcriptor), FBP (fusion sequence-based peptide), syn-B (derived from a natural mammalian antimicrobial peptide) and poly-arginine peptides.

Non-limiting examples of agents undergoing receptor-mediated transcytosis (RMT) include antibodies undergoing RMT, in particular antibody (fragments) specific for BBB receptors. Preferred examples of such antibodies include anti-transferrin receptor (TfR) antibodies, such as OX-26 (for example, as described in Friden P M, Walus L R, Musso G F, Taylor M A, Malfroy B, Starzyk R M. Anti-transferrin receptor antibody and antibody drug conjugates cross the blood-brain barrier. Proc Natl Acad Sci USA 1991; 88:4771-5); anti-insulin receptor (InsR) antibodies, such as 83-14 (for example as described in Pardridge W M, Kang Y S, Buciak J L, Yang J. Human insulin receptor monoclonal antibody undergoes high affinity binding to human brain capillaries in vitro and rapid transcytosis through the blood-brain barrier in vivo in the primate. Pharm Res 1995; 12: 807-16; Boado R J, Zhang Y, Zhang Y, Pardridge W M. Humanization of antihuman insulin receptor antibody for drug targeting across the human blood-brain barrier. Biotechnol Bioeng 2007; 96:381-91); anti-low-density lipoprotein receptor-related protein 1 (Lrp1) antibodies; anti-low-density lipoprotein receptor-related protein 2 (Lrp2) antibodies; antibodies against basigin; antibodies against Glutl; antibodies against CD98hc; and single domain (sd) antibodies directed against BBB surface receptors, such as FC5 and FC44 (for example, as described in Muruganandam A, Tanha J, Narang S, Stanimirovic D. Selection of phage-displayed llama single-domain antibodies that transmigrate across human blood-brain barrier endothelium. FASEB J Off Publ Fed Am Soc Exp Biol 2002; 16: 240-2; Abulrob A, Sprong H, Van Bergen en Henegouwen P, Stanimirovic D. The blood-brain barrier transmigrating single domain antibody: mechanisms of transport and antigenic epitopes in human brain endothelial cells. J Neurochem 2005; 95:1201-14).

Further preferred examples of agents undergoing receptor-mediated transcytosis (RMT) include transferrin, CRM197 (a non-toxic mutant of diphtheria toxin), and agents targeting low-density lipoprotein receptor related proteins, such as melanotransferrin, receptor-associated protein, p97 (for example as described in Karkan D, Pfeifer C, Vitalis T Z, Arthur G, Ujiie M, Chen Q, et al. A unique carrier for delivery of therapeutic compounds beyond the blood-brain barrier. PLoS One 2008; 3:e2469), LRP binding domain of the apolipoprotein B and angiopep-2 (AN-2) (for review see Yu Y. J. and Watts R. J. (2013) Developing therapeutic antibodies for neurodegenerative disease Neurotherapeutics 10:459-472).

In some embodiments, the agent facilitating the passage across the blood-brain barrier may be angiopep-2 (AN-2). AN-2 is a 19-mer peptide associating with the LRP1 receptor on blood-brain barrier capillary endothelial cells and enters the brain via RMT.

Conjugation of the antibody and the agent facilitating the passage across the blood-brain barrier is not particularly limited and may be achieved by any appropriate method known to the skilled person. Preferably conjugation is achieved directly or via one or more linker agents. Conjugation may be obtained by covalent linkage.

An example for conjugating an agent facilitating passage across the blood-brain barrier and an antibody is described in Regina A. et al. (2015) ANG4043, a Novel Brain-Penetrant Peptide-mAb Conjugate, Is Efficacious against HER2-Positive Intracranial Tumors in Mice. Mol Cancer Ther 14(1): 129-140. Accordingly, the antibody or the antigen-binding fragment thereof may be conjugated, for example to AN-2 as described by Regina et al., in particular as shown in FIG. 1A of Regina et al.

Compositions and Kits

The present invention also provides a composition comprising one or more of:
(i) the antibody of the present invention, or an antigen-binding fragment thereof;
(ii) the nucleic acid or the combination of nucleic acids of the present invention;
(iii) the vector or the combination of vectors of the present invention;
(iv) the cell expressing the antibody according to the present invention or comprising the vector according to the present invention; and/or
(v) the immunoconjugate of the present invention.

The composition may be used for treatment or diagnostic purposes. Accordingly, the composition may be a pharmaceutical composition or a diagnostic composition. The composition may comprise a (pharmaceutically acceptable) excipient, diluent or carrier.

Accordingly, the present invention also provides a pharmaceutical composition comprising an antibody according to the present invention, a nucleic acid according to the present invention, a vector according to the present invention, a cell according to the present invention, and/or an immunoconjugate according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition is not an active component in respect to TDP-43 proteinopathies.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody as described herein. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions may generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies as described herein. For example, the vehicle may be a physiologically acceptable liquid, which is suitable for storing, transporting, and/or administering a pharmaceutically active compound, in particular the antibodies as described herein. Once formulated, the compositions can be administered directly to the subject. In some embodiments the compositions are adapted for administration to mammalian, e.g., human subjects.

The pharmaceutical compositions may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. In some embodiments, the pharmaceutical composition may be administered into the central nervous system (CNS), e.g. the pharmaceutical composition may be administered via intracranial and intrathecal injections or using other administration routes for administration into the CNS, such as intracerebroventricular administration. Hyposprays may also be used to administer the pharmaceutical compositions. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound that is to be given to an individual, administration is usually in an “effective amount”, e.g. in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition may be provided for example in a pre-filled syringe.

The pharmaceutical composition may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the antibody as defined above, is combined with emulsifying and suspending agents. If desired, certain sweetening, flavoring or coloring agents may also be added.

The pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the pharmaceutical composition may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition can be formulated in a suitable lotion or cream. Suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as a single-dose product. In some embodiments, the amount of the antibody in the pharmaceutical composition—in particular if provided as a single-dose product—does not exceed 200 mg, for example it does not exceed 100 mg or 50 mg.

For a single dose, e.g. a daily, weekly or monthly dose, the amount of the antibody in the pharmaceutical composition, may not exceed 1 g or 500 mg. In some embodiments, for a single dose, the amount of the antibody in the pharmaceutical composition, may not exceed 200 mg, or 100 mg. For example, for a single dose, the amount of the antibody in the pharmaceutical composition, may not exceed 50 mg.

Pharmaceutical compositions typically include an “effective” amount of one or more antibodies as described herein, i.e. an amount that is sufficient to treat, ameliorate, attenuate, decrease or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. An effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody (e.g. amount of the antibody in the pharmaceutical composition) in relation to the bodyweight (e.g., in kg) of the individual to which it is administered.

Moreover, the pharmaceutical composition may also comprise an additional active component, which may be a further antibody or a component, which is not an antibody. Accordingly, the pharmaceutical composition may comprise one or more of the additional active components.

The antibody can be present either in the same pharmaceutical composition as the additional active component or, alternatively, the antibody may be comprised by a first pharmaceutical composition and the additional active component may be comprised by a second pharmaceutical composition different from the first pharmaceutical composition. Accordingly, if more than one additional active component is envisaged, each additional active component and the antibody may be comprised in a different pharmaceutical composition. Such different pharmaceutical compositions may be administered either combined/simultaneously or at separate times or at separate locations (e.g. separate parts of the body), optionally by different routes of administration.

The antibody and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In other embodiments, the pharmaceutical composition may not comprise an additional active component (in addition to the antibody of the invention or respective nucleic acids, vectors or cells as described above).

In some embodiments, the composition may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition, the antibodies may be in purified form.

The present invention also provides a method of preparing a pharmaceutical composition comprising the steps of: (i) preparing an antibody of the invention; and (ii) admixing the purified antibody with one or more pharmaceutically acceptable excipients, diluents or carriers.

In other embodiments, a method of preparing a pharmaceutical composition comprises the step of: admixing an antibody with one or more pharmaceutically-acceptable carriers, wherein the antibody is a monoclonal antibody.

As an alternative to delivering antibodies for therapeutic purposes, it is possible to deliver nucleic acid (typically DNA) that encodes the monoclonal antibody of interest to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions may also comprise one or more immunoregulatory agents. In some embodiments, one or more of the immunoregulatory agents include(s) an adjuvant.

The present invention also provides a diagnostic composition comprising an antibody according to the present invention, a nucleic acid(s) according to the present invention, a vector(s) according to the present invention, a cell according to the present invention, and/or an immunoconjugate according to the present invention. The diagnostic composition may optionally comprise suitable means for detection, such as reagents conventionally used in immuno- or nucleic acid based diagnostic methods.

The antibodies described herein are, for example, suited for diagnostic purposes. Accordingly, they may be used in immunoassays, in which they can be utilized in liquid phase or bound to a solid phase carrier. Such immunoassays may be competitive or non-competitive immunoassays; in either a direct or in an indirect format. Examples of such immunoassays include, but are not limited to, radioimmunoassay (RIA), enzyme-linked immunoassay (ELISA), sandwich (immunometric assay), immunohistochemistry, flow cytometry and Western blot assay. To this end, the antibody may be labelled, e.g. as described above.

In a further aspect the present invention also provides a kit comprising one or more containers containing one or more of
(i) the antibody according to the present invention, or an antigen-binding fragment thereof, as described above,
(ii) the nucleic acid molecule (or the combination of nucleic acid molecules) according to the present invention as described above,
(iii) the vector (or the combination of vectors) according to the present invention as described above,
(iv) the cell according to the present invention as described above,
(v) the immunoconjugate according to the present invention as described above; and/or
(vi) the composition according to the present invention as described above.

In addition, the kit may comprise means for administration of the antibody, or an antigen binding fragment thereof, according to the present invention, the nucleic acid according to the present invention, the vector according to the present invention, the cell according to the present invention or the pharmaceutical composition according to the present invention, such as a syringe or a vessel, a leaflet, and/or a co-agent to be administered as described herein. For example, the kit may contain a leaflet, e.g. comprising instructions for use. In addition or alternatively, the kit may comprise one or more reagents, e.g. for use in appropriate diagnostic assays. In some instances, the kit may contain a reference agent or control. In some embodiments, the composition of the invention may be provided in kit form, e.g., designed such that a combined composition is reconstituted just prior to administration to a subject. For example, a lyophilized antibody may be provided in kit form with sterile water or a sterile buffer (e.g., in a separate container).

Medical Treatments and Other Uses

In a further aspect, the present invention provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the immunoconjugate according to the present invention or the pharmaceutical composition according to the present invention as a medicament. In particular, the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the immunoconjugate according to the present invention, or the pharmaceutical composition according to the present invention may be used in prophylaxis and/or treatment of a TDP-43 proteinopathy; or in (ii) diagnosis of a TDP-43 proteinopathy.

Accordingly, the present invention also provides a method of treating, ameliorating or reducing a TDP-43 proteinopathy, or lowering the risk of (occurrence of) a TDP-43 proteinopathy, comprising: administering to a subject (in need thereof), (a therapeutically effective amount of) an antibody, or an antigen-binding fragment thereof, according to the present invention, a nucleic acid molecule (or the combination of nucleic acid molecules) according to the present invention, a vector (or the combination of vectors) according to the present invention, a cell according to the present invention, a immunoconjugate according to the present invention or a pharmaceutical composition according to the present invention. The present invention also provides a method for increasing soluble TDP-43 and/or decreasing phosphorylated TDP-43, e.g. in a TDP-43 pathology, the method comprising: administering to a subject (in need thereof), (a therapeutically effective amount of) an antibody, or an antigen-binding fragment thereof, according to the present invention, a nucleic acid molecule (or the combination of nucleic acid molecules) according to the present invention, a vector (or the combination of vectors) according to the present invention, a cell according to the present invention, a immunoconjugate according to the present invention or a pharmaceutical composition according to the present invention. Moreover, the present invention also provides the use of an antibody according to the present invention, or an antigen-binding fragment thereof, a nucleic acid molecule (or the combination of nucleic acid molecules) according to the present invention, a vector (or the combination of vectors) according to the present invention, a cell according to the present invention, an immunoconjugate according to the present invention, or a pharmaceutical composition according to the present invention in the manufacture of a medicament for prophylaxis, treatment or attenuation of a TDP-43 proteinopathy.

As used herein, the terms “treat” or “treatment” include therapeutic treatment and prophylactic or preventative measures. Prophylaxis of a TDP-43 proteinopathy refers in particular to prophylactic settings, wherein the subject was not diagnosed with a TDP-43 proteinopathy (either no diagnosis was performed or diagnosis results were negative) and/or the subject does not show symptoms of a TDP-43 proteinopathy. In therapeutic settings, in contrast, the subject is typically diagnosed with a TDP-43 proteinopathy and/or showing symptoms of a TDP-43 proteinopathy. Of note, the terms “treatment” and “therapy”/“therapeutic” of a TDP-43 proteinopathy include (complete) cure as well as attenuation/reduction of a TDP-43 proteinopathy and/or related symptoms.

In general, the object of the “treatment” may be to decrease, ameliorate, inhibit, prevent or slow down (lessen or delay) an undesired physiological change or disorder, such as the development of dementia. Beneficial or desired clinical results of a treatment include, but are not limited to, alleviation of symptoms, diminishment of the extent of a disease, stabilized (i.e., not worsening) state of disease, delay or slowing of disease progression, amelioration or palliation of the disease state, and remission (whether partial or total), whether detectable or undetectable. “Treatment” can also mean prolonging survival, e.g. as compared to expected survival if not receiving treatment. Those in need of treatment include those already with the condition or disorder as well as those prone to have the condition or disorder or those in which the manifestation of the condition or disorder or the risk thereof is to be decreased, delayed or prevented.

In some embodiments the subject may be a human. One way of checking efficacy of therapeutic treatment involves monitoring disease symptoms after administration of the antibody or of the composition. Treatment can be a single dose schedule or a multiple dose schedule. In some embodiments, an antibody, antibody fragment, nucleic acid, vector, cell, immunoconjugate or composition as described herein may be administered to a subject in need of such treatment. Such a subject includes, but is not limited to, one who is particularly at risk of, or susceptible to, a TDP-43 proteinopathy.

As used herein, the expression “TDP-43 proteinopathy” relates to a heterologous group of disorders characterized by the presence of pathological forms (abnormal species) of TDP-43, including extracellular, cytoplasmic and neuritic TDP-43, as well as to “TDP-43 oligomers”, “TDP-43 inclusion bodies” and “TDP-43 (high molecular weight) aggregates”, wherein TDP-43 forms fibril-like clumps. Diseases/disorders with TDP-43 proteinopathy are usually diseases of the nervous system diseases, in particular neurodegenerative diseases.

Examples of TDP-43 proteinopathies include, but are not limited to, frontotemporal lobar degeneration (FTLD), amyotrophic lateral sclerosis (ALS), argyrophilic grain disease, Alzheimer's disease, ALS-Parkinsonism dementia complex of Guam, corticobasal degeneration, dementia with Lewy bodies, Huntington's disease, Lewy body disease, motor neuron disease, frontotemporal dementia, hippocampal sclerosis, inclusion body myopathy, inclusion body myositis, Parkinson's disease, Parkinson's disease dementia, Parkinson-dementia complex in Kii peninsula, Pick's disease, Machado-Joseph disease and the like, as described, for example, in Lagier-Tourenne et al. Hum. Mol. Gen. 19 (2010), R46-64, which is herein incorporated by reference in its entirety. While TDP-43 predominantly localizes to the nucleus under normal physiological conditions, a substantial loss of nuclear TDP-43 is typically observed in neurons bearing aberrant cytoplasmic TDP-43 inclusions. TDP-43 exhibits a disease-specific biochemical signature; pathologically altered TDP-43. TDP-43 proteinopathies can usually be distinguished from most other neurodegenerative disorders in which protein misfolding leads to brain amyloidosis, because pathologic TDP-43 forms neuronal and glial inclusions lacking the features of brain amyloid deposits.

Therefore, the TDP-43 proteinopathy may be selected from the group consisting of frontotemporal lobar degeneration (FTLD), amyotrophic lateral sclerosis (ALS), argyrophilic grain disease, Alzheimer's disease, ALS-Parkinsonism dementia complex of Guam, corticobasal degeneration, dementia with Lewy bodies, Huntington's disease, Lewy body disease, motor neuron disease, frontotemporal dementia, hippocampal sclerosis, inclusion body myopathy, inclusion body myositis, Parkinson's disease, Parkinson's disease dementia, Parkinson-dementia complex in Kii peninsula, Pick's disease and Machado-Joseph disease. Preferably, the TDP-43 proteinopathy is FTLD or ALS.

Accordingly, an antibody according to the present invention, or an antigen-binding fragment thereof, a nucleic acid molecule (or the combination of nucleic acid molecules) according to the present invention, a vector (or the combination of vectors) according to the present invention, a cell according to the present invention, an immunoconjugate according to the present invention or the pharmaceutical composition according to the present invention may be used to treat a neurological disorder characterized by abnormal accumulation and/or deposition of TDP-43 in the brain and the central nervous system.

The antibody according to the present invention, or an antigen-binding fragment thereof, the nucleic acid molecule (or the combination of nucleic acid molecules) according to the present invention, the vector (or the combination of vectors) according to the present invention, the cell according to the present invention, the immunoconjugate according to the present invention or the pharmaceutical composition according to the present invention may be administered by any route of administration including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. In some embodiments, the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the immunoconjugate according to the present invention or the pharmaceutical composition according to the present invention may be administered into the central nervous system (CNS), e.g. via intracranial and intrathecal injections or using other administration routes for administration into the CNS, such as intracerebroventricular administration. In addition, any gene therapy approaches may be used, e.g. the antibody according to the present invention, or an antigen-binding fragment thereof, may be administered as nucleic acid or vector encoding said antibody, e.g. using viral or non-viral vectors as described above. In some embodiments, the antibody according to the present invention, or an antigen-binding fragment thereof, the nucleic acid molecule (or the combination of nucleic acid molecules) according to the present invention, the vector (or the combination of vectors) according to the present invention, the cell according to the present invention, the immunoconjugate according to the present invention or the pharmaceutical composition according to the present invention may be administered systemically, for example by intravenous or subcutaneous administration.

In some embodiments, co-administration or sequential administration of (i) an antibody according to the present invention, or an antigen-binding fragment thereof, a nucleic acid molecule (or the combination of nucleic acid molecules) according to the present invention, a vector (or the combination of vectors) according to the present invention, a cell according to the present invention, an immunoconjugate according to the present invention or the pharmaceutical composition according to the present invention and (ii) a co-agent, which may be a neuroprotective agent useful for treating a TDP-43 proteinopathy may be desirable. In some instances, the co-agent may be comprised in the pharmaceutical composition. Examples of neuroprotective agents useful in the context of the present invention include, but are not limited to, an acetylcholinesterase inhibitor, a glutamatergic receptor antagonist, kinase inhibitors, HDAC inhibitors, anti-inflammatory agents, divalproex sodium, or any combination thereof. In some embodiments, the co-agent is dopamine or a dopamine receptor agonist. Examples of other neuroprotective agents that can be used as co-agents are known and described in the art, for example in WO2007/011907, which is incorporated herein by reference.

Antibodies and fragments thereof as described herein, as well as the immunoconjugates as described above, may also be used for the (in-vitro) diagnosis of a TDP-43 proteinopathy. Methods of diagnosis may include contacting an antibody with a sample. Such samples may be isolated from a subject, for example an isolated tissue sample taken from, for example, nervous tissue, in particular brain or spinal cord tissue, cerebrospinal fluid (CSF), nasal passages, sinus cavities, salivary glands, lung, liver, pancreas, kidney, ear, eye, placenta, alimentary tract, heart, ovaries, pituitary, adrenals, thyroid, brain, skin or blood, such as whole blood, plasma or serum. In some embodiments, the antibody, or an antigen-binding fragment thereof, may be contacted with an (isolated) CSF sample or with an (isolated) nervous tissue sample. The methods of diagnosis may also include the detection of an antigen/antibody complex, in particular following the contacting of an antibody with a sample. Such a detection step is typically performed in vitro, i.e. without any contact to the human or animal body. Examples of detection methods are well-known to the person skilled in the art and include immunoassays such as flow cytometry, dot or slot blots, Western blots, ELISA (enzyme-linked immunosorbent assay), immunohistochemistry and immunoprecipitation followed by SDS-PAGE immunocytochemistry. The level of such antigen/antibody complexes may then be determined by methods known in the art, optionally, and may be compared to a control sample. A level (significantly) higher than that of the control may indicate the disease in the tested individual. Thus, the invention relates to an in vitro immunoassay comprising antibody or antigen-binding fragment thereof of the invention. In some embodiments, a labelled antibody, in particular an immunoconjugate as described above, may be used in such an approach. A labelled antibody, in particular an immunoconjugate, may also be useful to detect the location of TDP-43.

Accordingly, the diagnosis may be performed in vitro, for example by using an isolated sample as described above (and an in vitro detection step of an antigen/antibody complex). In summary, the antibody, or an antigen-binding fragment thereof, as well as the immunoconjugate as described above, may be used in (in vitro) diagnosis of a TDP-43 proteinopathy.

The present invention also provides a method of detecting whether a sample comprises pathological TDP-43, in particular a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated. Such a method may comprise the following steps:
a. contacting the sample with the antibody described herein under conditions permissive to produce an antibody/antigen complex; and
b. detecting the presence of the antibody/antigen complex.

Thereby, the presence of detectable antibody/antigen complex may be indicative that the sample may contain pathological TDP-43, in particular a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated.

In some embodiments, in step (b) the amount of the antigen/antibody complex may be determined (e.g., measured) in the test sample. Thereafter, said amount may be compared to a control.

Accordingly, the antibody of the present invention, or an antigen-binding fragment thereof, as well as the immunoconjugate as described above, may be used in an (in vitro) method for detecting pathological TDP-43. Likewise, the antibody of the present invention, or an antigen-binding fragment thereof, may be used in an (in vitro) method for binding pathological TDP-43, such as (phosphorylated aggregates and/or C-terminal fragments of TDP-43, for example in the cytoplasm or neurites of nervous tissue (e.g. from the brain or spinal cord). Due to its specificity, the antibody of the present invention, or an antigen-binding fragment thereof, specifically recognizes pathological TDP-43, e.g. as observed in various TDP-43 proteinopathies. For detecting pathological TDP-43, the antibody may be brought in contact with a (isolated) sample (i.e., a sample to be tested for the presence of the antigen). By the specific binding of the antibody to its antigen (pathological TDP-43), an antibody/antigen complex is formed, which can be easily detected by methods known in the art. In some embodiments, (in vitro) detection of pathological TDP-43 may be performed post mortem (e.g. on an isolated brain or spinal cord sample).

Such a detection method may be used in the context of (in vitro) diagnosis (with samples isolated from a human or animal body), but also for testing other (e.g., production/manufacture) samples, such as immunogenic composition or vaccine samples. Accordingly, antibodies, antibody fragment, or variants thereof, as described in the present invention, as well as the immunoconjugates as described above, may also be used in a non-therapeutic/non-diagnostic context, e.g. in development or manufacture of immunogenic compositions (vaccines). The present invention therefore also provides the use of the antibody of the present invention, or an antigen-binding fragment thereof, as well as the immunoconjugates as described above, for testing immunogenic compositions/vaccines, in particular of an immunogenic composition/vaccine comprising a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated. The specific binding of the antibody to TDP-43 and its C-terminal fragments phosphorylated at S403/S404 enables testing of respective TDP-43 vaccines, whether they are phosphorylated at S403/S404 (e.g., to mimic pathological TDP-43). Accordingly, the antibodies may be used for monitoring immunogenic composition/vaccine manufacture with the desired immunogenicity. To this end, the antibody may be brought in contact with the immunogenic composition/vaccine, e.g. as described above.

Accordingly, the present invention also provides a method for testing immunogenic compositions (vaccines) based on pathological TDP-43, wherein the immunogenic composition/vaccine is contacted with the antibody, or an antigen-binding fragment thereof, and, optionally, the presence of antibody/antigen complexes is determined. Furthermore, the present invention also encompasses the use of the antibody of the present invention, or an antigen-binding fragment thereof, for monitoring the quality of immunogenic compositions/vaccines based on pathological TDP-43 by checking whether the immunogenic composition/vaccine contains the desired antigen, e.g. a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated. More specifically, the antibody may be used to check the phosphorylation pattern of the antigen, or a fragment thereof, in an immunogenic composition/vaccine. As the antibodies of the present invention bind specifically to the phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated, detection of a significant amount of antibody/antigen complexes in a sample may imply that the sample contains pathological TDP-43. To assess conformation specific binding, for example surface plasmon resonance (SPR) may be used.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 the reactivity plot for the detection of pTDP-43-specific antibodies in sera of a large unselected donor population obtained by high-throughput ELISA. The box includes positive sera to TDP-43 pS403/404.

FIG. 2 shows for Example 2 EC50 ELISA determination of binding of exemplary anti-pS403/404 TDP-43 antibody 31F3 to pS403/404 TDP-43 and pS409/410 TDP-43 peptides (Schafer-N), recombinantly expressed full-length TDP-43 (OriGene Technologies, Rockville, MD, USA). Bovine serum albumin (BSA) serves as control. Similar binding was detected for the antibodies 30E1, 9F11, 15D7.

FIG. 3 shows for Example 3 surface plasmon resonance (SPR) affinity data for binding of antibodies 31F3, 30E1 and 9F11 to (A) pS403/404 TDP-43 (“peptide 2”); and (B) pS409/410 TDP-43 peptides (“peptide 1”).

FIG. 4 shows for Example 4 a Western Blot using SarkoSpin pellets from post-mortem brain tissue of FTLD patients and healthy controls, which shows binding of 31F3 antibody to insoluble TDP-43 aggregates and C terminal fragment of 25 kDa (CTF25).

FIG. 5 shows for Example 5 that immunofluorescence of motor neuron-like NSC-34 cells overexpressing GFP-TDP-43 reveals co-localization of 31F3 antibody with pathological cytoplasmic TDP-43 accumulations.

FIG. 6 shows for Example 6 that immunofluorescence of brain sections (frontal cortex) of FTLD patients with TDP-43 pathology shows co-localization of 31F3 antibody with neuronal cytoplasmic inclusions (large arrows; upper panel) and dystrophic neurites (large arrows; lower panel). Nuclear TDP-43 in neighboring non-affected cells is depicted by small arrows and is not detected by 31F3.

FIG. 7 shows for Example 7 the workflow of anti-pTDP-43 antibody testing in a HEK293 seeding model. 24 h after plating, TDP-43-HA expression was induced by addition of doxycycline (DOX). 48 h after plating, cell mitosis was blocked using AraC and cells were transfected with SarkoSpin (SKS) fraction of post-mortem brain samples obtained from patients with FTLD-TDP type A (FTLD-A) or C (FTLD-C) and non-neurodegenerative control. Before transfection, the SKS fraction was incubated with 31F3, IgG control or PBS control in a ratio 1:5 of SKS fraction to antibody for 2 h at room temperature (RT). Cells were then fixed 3 to 6 days post-transfection and used for western blot analysis.

FIG. 8 shows for Example 7 the characterization of the seeding properties of patient-derived pathological TDP-43 in HEK293 cells. HEK293 stable cells expressing TDP-43-HA under doxycycline induction were seeded with pathological TDP-43 from post-mortem brains obtained from patients with FTLD-TDP type A (FTLD-A) or C (FTLD-C) and non-neurodegenerative control. 24 hrs after doxycycline induction, cells were mitotically arrested (using AraC), inoculated with SarkoSpin pellets using Lipofectamine 2000 and monitored for up to 6 days. (A) Graph showing the quantification of neo-aggregates (NeoAgg) overtime, showing their increase upon incubation with FTLD-TDP-A and FTLD-TDP-C, compared to non-degenerative brain sample and non-transfected AraC control. 2-way ANOVA with post-hoc Fisher's LSD, F(4,308)=11.38, P<0.0001, NS difference for Day 3, 4 and 5. Day 6: AraC vs FTLD-TDP-A p=0.006, AraC vs FTLD-TDP-C p=0.0075, Control vs FTLD-TDP-A p=0.0009, Control vs FTLD-TDP-C p=0.0014. (B) Western blots showing increase of sarkosyl-insoluble TDP-43-HA aggregates (pellet) in seeded HEK293 cells with brain material from FTLD-TDP-A and FTLD-TDP-C, compared to non-degenerative brain sample control. The insoluble TDP-43-HA is increased overtime. Heading D3-6: indicates the number of days post-inoculation. Results are representative from several patients n=3 per group.

FIG. 9 shows for Example 7 that neo-aggregates (NeoAgg) of HEK293 cells transfected with FTLD-TDP-A or FTLD-TDP-C co-localize with antibody 31F3 detecting pTDP-43. Imaris surface masks of seeded HEK293 cells after 6 days shows DAPI (marked outline), TDP-43-HA (upper panel, light grey), pTDP-43 (detected with antibody 31F3, middle panel, light grey) and neo-aggregates (NeoAgg, co-localization between pTDP-43 and TDP-43-HA, lower panel, light grey). The high-resolution images reveal presence of large cytoplasmic NeoAgg in HEK293 cells seeded with FTLD-TDP-A and multiple, smaller cytoplasmic NeoAggs seeded with FTLD-TDP-C.

FIG. 10 shows for Example 7 that pre-treatment of patient-derived brain samples with 31F3 antibody decreased insoluble TDP-43-HA in seeded HEK cells. Western blot of HEK293 cells transfected with patient-derived pathological TDP-43 from FTLD-TDP-A (FTLD-A), FTLD-TDP type C (FTLD-C) and non-degenerative brain control. Before transfection, the SKS was incubated in a ratio 1:5 of SKS to antibody for 2 h at room temperature with 31F3, IgG control and PBS control (untreated) (See workflow in FIG. 7). Treatment with 31F3 antibody decreased insoluble TDP-43-HA in seeded HEK cells.

FIG. 11 shows for Example 8 specific detection of TDP-43 inclusions with antibody 31F3 in ALS, namely, thoracic spinal cord from 75-year-old female diagnosed with ALS 4 years prior to death. Arrow: glial intracytoplasmatic inclusion. Arrow head: dystrophic neurites. Asterisk: motor neurons.

FIG. 12 shows for Example 8 specific detection of TDP-43 inclusions with antibody 31F3 in Alzheimer's disease pathology with Granulovacuolar degeneration, namely, Granulovacuolar degeneration (GVD) subiculum of 58 year old male with incidental finding of Alzheimer's disease pathology with GVD (Stage 1 according to Thal D R et al. Stages of granulovacuolar degeneration: their relation to Alzheimer's disease and chronic stress response. Acta Neuropathol. 2011 November; 122(5):577-89. doi: 10.1007/s00401-011-0871-6). Arrow: granulovacuolar degeneration.

FIG. 13 shows for Example 8 8 specific detection of TDP-43 inclusions with antibody 31F3 in limbic predominant age-related TDP-43 encephalopathy (LATE), namely, 91 year old male with clinical suspicion of Alzheimer's disease vs. vascular dementia/diagnosis at autopsy: limbic-predominant age-related TDP-43 encephalopathy (LATE, Nelson P T, et al. Limbic-predominant age-related TDP-43 encephalopathy (LATE): consensus working group report. Brain. 2019 Jun. 1; 142(6):1503-1527). (A) Diseased area: subiculum. Arrow: neuronal intracytoplasmatic inclusion. Arrow head: dystrophic neurites. (B) Control area: dentate gyrus and CA4. Arrow: granule neurons, dentate gyrus. Arrow head: pyramidal cells, CA4.

FIG. 14 shows for Example 10 immunodepletion from brain samples. Brain lysates from FTLD patients were immunoprecipitated with 31F3, 30E1, 9F11 antibodies and an isotype control. Supernatants after immunoprecipitation (immunodepleted sample) were loaded on a gel and showed successful depletion of pTDP (S403/404) (A) as well as depletion of total TDP-43 (B) and pTDP-43 (S409/410) (C) showing that the antibodies are able to deplete all TDP-43 aggregated species from patient brain material. As a control of successful immunoprecipitation, the presence of TDP-43 in the bead immunoprecipitated sample with the anti-TDP-43 antibodies is shown (D).

FIG. 15 shows for Example 11 that antibodies 30E1 and 31F3 can bind in the presence of pTDP-43 (S409/410). Antibodies 30E1 (A) and 31F3 (B) were captured on anti-human fc sensors on an Octet (step “1”). In step “2” sensors were then exposed to the TDP-43 peptide phosphorylated on both pS403/404 and pS409/410. Then, in step “3”, sensors were loaded with an pS409/410 TDP-43 antibody (Proteintech).

FIG. 16 shows for Example 12 framework engineered versions of the 31F3 (“MY001-31F3”) antibody, which were tested for binding to pS403/404 TDP-43 peptide by ELISA.

FIG. 17 shows for Example 13 the comparison of the 31F3 (“MY001-31F3”) antibody to a pS403/404 TDP-43 rabbit polyclonal antibody. HEK cells seeded with patient material were stained with 31F3 (“MY001-31F3”) or a commercially available pS403/404 TDP-43 rabbit polyclonal antibody (CosmoBio, Catalog No. TIP-PTDP-P05). Analysis of neoaggregate formation showed a lower number of detected aggregates with the pS403/404 TDP-43 rabbit polyclonal antibody. The pS403/404 TDP-43 rabbit polyclonal antibody was raised by immunizing a rabbit with the peptide NGGFGS(p)S(p)MDSKC as described in Hasegawa et al 2008.

FIG. 18 shows for Example 14 the comparison of antibodies of the invention (31F3 and 30E1) with a pS403/404 TDP-43 rabbit polyclonal antibody and a pS403/404 TDP-43 mouse monoclonal antibody. 31F3 (MY001-31F3; A), 30E1 (MY001-30E1; B), a pS403/404 TDP-43 rabbit polyclonal antibody (C), and a pS403/404 TDP-43 mouse monoclonal antibody (D) were tested for binding to different TDP-43 antigens as indicated in the figure by ELISA.

FIG. 19 shows for Example 15 that antibodies 30E1 and 31F3 reach the brain after systemic (i.p.) injection in a mouse model of TDP-43 pathology and bind to TDP-43 aggregates. Brain lysates from TDP43-ΔNLS mice induced for 5 weeks and treated with a single injection of 50 mg/Kg of 30E1 or 31F3 were tested for presence of the antibodies in the brain by ELISA (A). N=4 Mean and standard error of the mean. Analysis of the brain lysate by Sarkospin method to enrich for aggregates, showed presence of the antibodies in the TDP-43 insoluble aggregate fraction only in animals treated with anti-TDP-43 antibodies 31F3 and 30E1, proving target engagement of the antibodies (B). Individual values and mean and standard deviation, p-values from 2 way anova with post-hoc Fisher's LSD, ** p<0.01 * p<0.05 ns=non significant.

FIG. 20 shows for Example 15 the effect of treatment with antibodies 30E1 and 31F3 on soluble and insoluble TDP-43 in the brain. Brain lysates from TDP43-ΔNLS mice induced for 5 weeks and treated with a single injection of 50 mg/kg of 30E1 or 31F3 were subjected to Sarkospin method to separate aggregates from soluble proteins. The amount of pTDP-43 (409/410) and soluble TDP-43 was quantified by Western Blot and normalized on SOD1. The data show significant increase over vehicle in soluble TDP-43 and a decrease in phosphorylated TDP-43, indicating that antibodies 31F3 and 30E1 are able to counteract pathological aggregation of TDP-43.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Identification and Production of Antibodies Binding to TDP-43 pS403/404

Patient Population

To detect individuals harboring antibodies directed against pTDP-43 peptides, a large unselected population, with a clinical history unknown at the time of conducting the test, was screened for seroreactivity to pTDP-43 peptides in a high-throughput ELISA (see below: High-throughput ELISA for the detection pTDP-43-specific antibodies in sera of large unselected donor population). Following the identification of target patients with seroreactivity to pTDP-43 peptides, their lymphocytes were obtained from surplus material or isolated from a whole blood sample obtained during an additional blood donation of said patients. All patients whose lymphocytes were used provided a hospital-wide general consent and/or gave their written informed consent. The study has been approved by the ethical Committee of the canton of Zurich.

High-Throughput ELISA for the Detection pTDP-43-Specific Antibodies in Sera of Large Unselected Donor Population

High-binding 1536 well microplates (SpectraPlate 1536 HB, Perkin Elmer, Waltham, MA, USA) were coated with 0.5 μg/ml human pS403/404 TDP-43 (SEQ ID NO: 2, which corresponds to amino acids from position 391 to 414 of full-length human TDP-43 of SEQ ID NO: 1; phosphorylated at serine residues at positions 14 and 15 (“pS403/404 TDP-43 peptide”; “peptide 2”) of SEQ ID NO: 2, which correspond to the serine residues at positions 403 and 404 of SEQ ID NO: 1) or with other TDP-43 peptides synthetically produced by Schafer-N(Copenhagen, Denmark) in PBS at 37° C., similarly to reports using different proteins (Senatore, Assunta, Karl Frontzek, Marc Emmenegger, Andra Chincisan, Marco Losa, Regina Reimann, Geraldine Horny, et al. 2020. “Protective Anti-prion Antibodies in Human Immunoglobulin Repertoires.” EMBO Molecular Medicine 12 (9). https://doi.org/10.15252/emmm.202012739). Plates were washed with PBS-Tween 0.1% and blocked 1 h at room temperature with PBS-Tween 0.1% containing 5% Milk (Rapilait, Migros, Zurich, Switzerland). Patient plasma (at dilutions ranging from 1:50 to 1:600) were dispensed using an ECHO 555 Acoustic Dispenser (Labcyte, San José, CA, USA) and were incubated for 2 h at room temperature. Binding of human total IgG to pS403/404 TDP-43 was determined using a HRP-conjugated goat anti human Fc-gamma-specific antibody (Jackson ImmunoResearch, West Grove, PA, USA) followed by measurement of the HRP activity using a tetramethylbenzidine substrate solution (TMB, ThermoFisher Scientific, Carlsbad, CA, USA). Absorbance at 450 nm was measured (EnVision, Perkin Elmer) and the inflection points of the sigmoidal binding curves were determined using a fitting algorithm previously described (Emmenegger, Marc, Elena De Cecco, David Lamparter, Raphael P. B. Jacquat, Daniel Ebner, Matthias M Schneider, Itzel Condado Morales, et al. 2020. “Early Peak and Rapid Decline of SARS-CoV-2 Seroprevalence in a Swiss Metropolitan Region.” MedRxiv). Samples reaching half-maximum saturation (shown as the inflection point of the logistic regression curve) at a concentration ≤1:100, i.e. at −log(EC50)≥2, and with a mean squared residual error <20% of the actual −log(EC50) were considered hits.

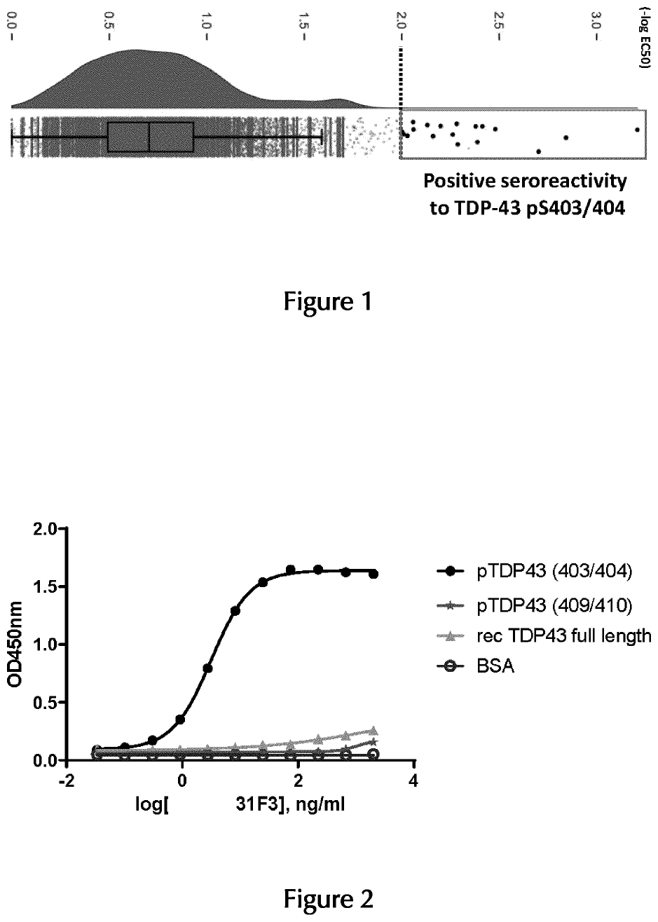

50′360 patient sera were screened as described above for the presence of autoantibodies against pS403/404 TDP-43. Overall, only a very low prevalence (0.04% for pS43 403/404 TDP-43) of positive sera has been detected, indicating the need to screen very large populations in order to identify functional antibodies. Results of the pS403/404 TDP-43 screen are shown in FIG. 1. FIG. 1 shows the reactivity plot for the detection of pTDP-43-specific antibodies in sera of a large unselected donor population obtained by high-throughput ELISA. The box includes positive sera to TDP-43 pS403/404.

Isolation of Peripheral Blood Mononuclear Cells (PBMC)

Lymphocytes from surplus material were collected from centrifuged whole blood. Therefore, the plasma fraction was removed and the lymphocyte fraction was collected and re-suspended in supplemented IMDM (Gibco, ThermoFisher Scientific, Waltham, MA, USA). In order to minimize erythrocyte contamination, lymphocytes were treated with erythrocyte lysis buffer. Afterwards, lymphocytes were frozen in FCS containing 10% dimethylsufloxide (DMSO, Sigma-Aldrich Chemie GmbH, Buchs, Switzerland). Peripheral blood mononuclear cells (PBMCs) were isolated from heparinized peripheral blood using Lympholyte H according to manufacturer's instruction (Cedarlane, Burlington, Ontario, Canada) and cryopreserved prior to use.

Isolation of Memory B Cells

Peripheral blood mononuclear cells (PBMCs) or lymphocytes were stained on ice with monoclonal antibodies phycoerythrin-conjugated anti-human IgD and IgA, APC-conjugated mAbs anti-human IgM, CD3, CD56, CD8, and FITC-conjugated mAb anti-human CD22 (Becton Dickinson, Basel, Switzerland). Cells sorting was carried out using a MoFlo XDP cell sorter (Beckman Coulter, Krefeld, Germany). CD22 positive and IgM, IgD, IgA negative B cells were seeded at 5-10 cells per well on irradiated CD40L-expressing feeder cells stimulated with a cytokine cocktail, as described in Huang et al., ‘Isolation of human monoclonal antibodies from peripheral blood cells’, Nature Protocols, 2012.

After 10-14 days of stimulation, culture supernatants were screened for the presence of IgG antibodies specific for the pTDP-43 peptides (pS403/404 TDP-43 and pS409/410 TDP-43 as described above) by ELISA. Detection of pTDP-43-specific IgG antibodies was performed using anti-human HRP-conjugated goat Fc-gamma-specific antibody (Jackson ImmunoResearch, West Grove, PA, USA) followed by measurement of the HRP activity using a tetramethylbenzidine substrate solution (TMB, Sigma-Aldrich Chemie GmbH, Buchs, Switzerland). Subsequently the antibody for which binding was detected or the cell producing said antibody was isolated.

Molecular Cloning of Human Antibodies Specific to pTDP-43 Peptides

Molecular cloning of human antibodies specific to pTDP-43 peptides was carried out according to Huang et al., ‘Isolation of human monoclonal antibodies from peripheral blood cells’, Nature Protocols, 2012. In particular, single cells obtained from pTDP43-reactive memory B cell cultures were sorted into a 96 well PCR plate, containing reverse-transcription buffer (Invitrogen, Carlsbad, CA, United States). cDNA preparation was done using Random Hexamer Primer (Invitrogen, Carlsbad, CA, United States) according to the supplier's protocol. Immunoglobulin heavy and light chain variable regions (V_{H} and V_{L}, respectively) were PCR amplified according to standard protocols (Wardemann et al, Science 301, 2003, 1374-1377). To increase the PCR efficiency, a nested PCR was performed. The first round PCR was performed with primers specific for the IgG constant region and primer mixes specific to all leader sequences from V_{H} and V_{L} families (Wardemann et al, Science 301, 2003, 1374-1377). Subsequently, a nested PCR was performed using primer mixes specific to the 5′ region of framework 1 of V_{H} and VL families (V-region) and the immunoglobulin J-regions. Sequence analysis was carried out to identify individual antibody clones present in the selected B cell culture. Afterwards, the VH and V_{L} of each unique antibody clone were cloned into expression vectors providing the constant regions of human IgG1, human Ig-Kappa or human Ig-Lambda. Upon co-transfection of the Ig-heavy- and light expression vectors into HEK293T cells, the respective antibody clones are produced. Identification of the antibody clone presumably responsible for the anti-pTDP-43 reactivity of the parental B cell culture was performed upon re-screening of the recombinant antibodies for reactivity to pTDP-43 peptides in an ELISA.

In order to identify and to correct primer encoded sequence mismatches in the Ig-variable regions, an additional PCR amplification on original cDNA of the reactive clone was performed using a semi-nested PCR. Therefore, primer mixes specific for the Ig heavy and light chain leader sequences (5′-primers) and two primer pairs specific for a conserved region of the Ig heavy and light chain constant regions (3′-primers) were used. Subsequently, PCR products were directly subjected to sequencing using internal primers specific to conserved regions of the constant domains of heavy and light chains. Sequence determination and annotation of the complete antibody was carried out and this information was used to design specific primers for the cloning of the authentic human V_{H} and V_{L} sequence into antibody (IgG) expression vectors. This approach also allows the identification of Ig isotype (and subclass) of each isolated, reactive monoclonal antibody. These V_{H} and V_{L} sequences were then used for the production of recombinant antibodies which were subsequently characterized in more detail.

Thereby, antibodies 31F3, 30E3 (also referred to herein as “30E1”), 9F11 and 15D7 were identified and selected for further characterization. V_{H} and VL sequences as well as CDR sequences of the exemplary antibodies identified as described above are shown in Table 3 below.

TABLE 3
SEQ ID NOs of sequences as well as CDR and V_{H}/V_{L}
sequences of exemplary antibodies of the invention.

| | CDRH1 | CDRH2 | CDRH3 | CDRL1 | CDRL2 | CDRL3 | VH | VL |
|---|---|---|---|---|---|---|---|---|
| 31F3 | 3 | 4 | 5 | 6 | 7 | 8 | 9 | 10 |
| 30E1 | 13 | 14 | 15 | 6 | 16 | 17 | 18 | 19 |
| 9F11 | 13 | 22 | 23 | 24 | 25 | 17 | 26 | 27 |
| 15D7 | 28 | 29 | 30 | 31 | 32 | 33 | 34 | 35 |

Antibody Production and Purification

Recombinant monoclonal human antibodies were expressed upon transfection of antibody-coding expression vectors into suspension HEK293 or Chinese Hamster Ovary (CHO) cells by the Polyethylenimine transfection method (PEI, Polyscience Warrington, USA). After transfection, cells were cultured 7 days. Subsequently, supernatants were collected and IgG-antibodies were purified using protein A columns (GE HealthCare, Sweden) on a fast protein liquid chromatography device (FPLC, ÄKTA, GE HealthCare, Sweden).

### Example 2: Binding of Selected Antibodies to Distinct TDP-43 Peptides

To investigate binding specificity, binding of the selected antibodies 31F3, 30E1, 9F11 and 15D7 to recombinant non-phosphorylated full-length TDP-43, to the phosphorylated TDP-43 peptide described in Example 1 (“peptide 2”: pS403/404 TDP-43), and to the phosphorylated TDP-43 “peptide 1”: pS409/410 TDP-43 (SEQ ID NO: 2, which corresponds to amino acids from position 391 to 414 of full-length human TDP-43 of SEQ ID NO: 1; phosphorylated at serine residues at positions 20 and 21 (“pS403/404 TDP-43 peptide”; “peptide 2”) of SEQ ID NO: 2, which correspond to the serine residues at positions 409 and 410 of SEQ ID NO: 1) was tested by ELISA.

To this end, 96 well microplates (Costar®, Corning Incorporated, Corning, NY, USA) were coated with synthetical pTDP-43 peptides (0.5 μg/ml pS403/404 TDP-43 or 0.5 μg/ml pS409/410 TDP-43, Schafer-N) or with non-phosphorylated full-length TDP-43 (SEQ ID NO: 1). Plates were washed with PBS-Tween 0.05% and blocked 1 h at room temperature with PBS containing 5% Milk (Rapilait, Migros, Zurich, Switzerland) or 2% bovine serum albumin (BSA, Sigma-Aldrich Chemie GmbH, Buchs, Switzerland). Patient sera, B cell conditioned medium, or recombinant antibody preparations were incubated for 2 h at room temperature. Binding of human IgG to the antigen of interest was determined using a HRP-conjugated anti human antibody (anti-IgG-HRP from Jackson ImmunoResearch, West Grove, PA, USA) followed by measurement of the HRP activity using a tetramethylbenzidine substrate solution (TMB, Sigma-Aldrich Chemie GmbH, Buchs, Switzerland).

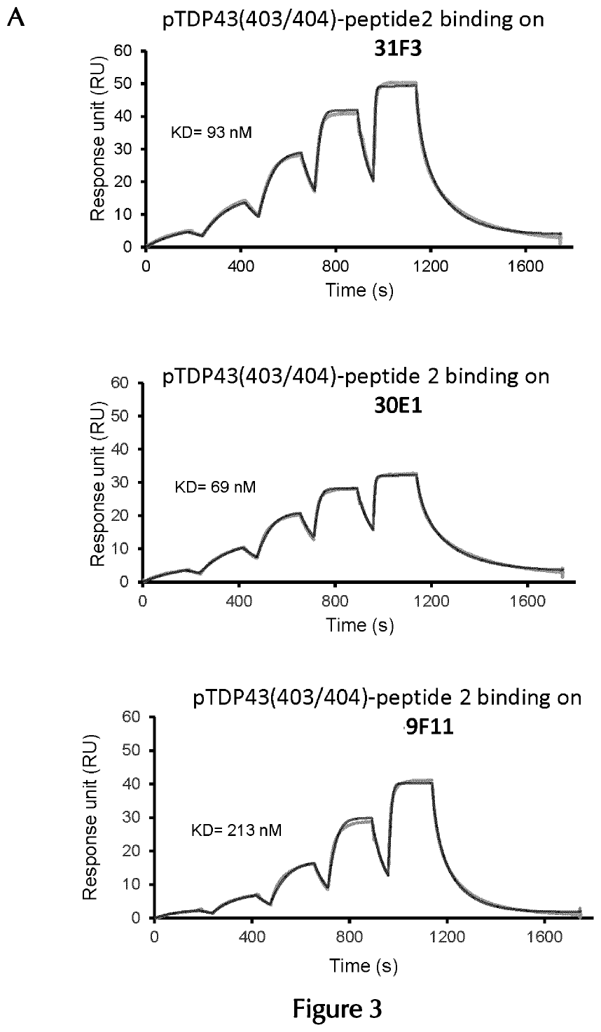

Results for the exemplary antibody 31F3 are shown in FIG. 2. Similar binding was also detected for the antibodies 30E1, 9F111 and 15D7. These data show that the antibodies are specific for pathological TDP-43 phosphorylated at positions S403 and S404. For non-pathological full-length TDP-43 and for TDP-43 phosphorylated at positions S409 and S410 no binding could be detected, indicating the high specificity and selectivity of the antibodies for pTDP-43 (403/404).

### Example 3: SPR Binding Study of Selected Antibodies

Next, the binding properties of antibodies 31F3, 30E1 and 9F11 was studied using surface plasmon resonance (SPR).

Briefly, binding properties (ka, kd and KD) of selected antibodies with respect to pTDP-43 were determined at 25° C. using a real-time biosensor surface plasmon resonance assay (BIACORE™ 8K), using 10 mM HEPES pH 7.4, 150 mM NaCl, 3 mM EDTA and 0.005% Tween-20 as running buffer. Antibodies were immobilized on the surface of CM5 sensor chip at 100 nM concentration through standard amine coupling. To test pTDP-43 binding, the two pTDP-43 peptides as described above (corresponding amino acids from position 391 to 414 of human TDP-43 of SEQ ID NO: 1) were synthesized (Schafer-N) with distinct phosphorylation at sites 409/410 (peptide 1) or sites 403/404 (peptide 2) (cf. above: pS403/404 TDP-43 and pS409/410 TDP-43). Increasing concentrations (12-37-111-333-1000 nM) of these peptides were injected over the captured antibody surface at 30 μl/min for 180 sec to measure association rates (ka). Dissociation rate (kd) was monitored for 600 sec. Analyte responses were corrected for unspecific binding and buffer responses. Curve fitting and data analysis to determine the kinetic parameters, were performed with Biacore™ Insight Evaluation Software v2.0.15.12933.

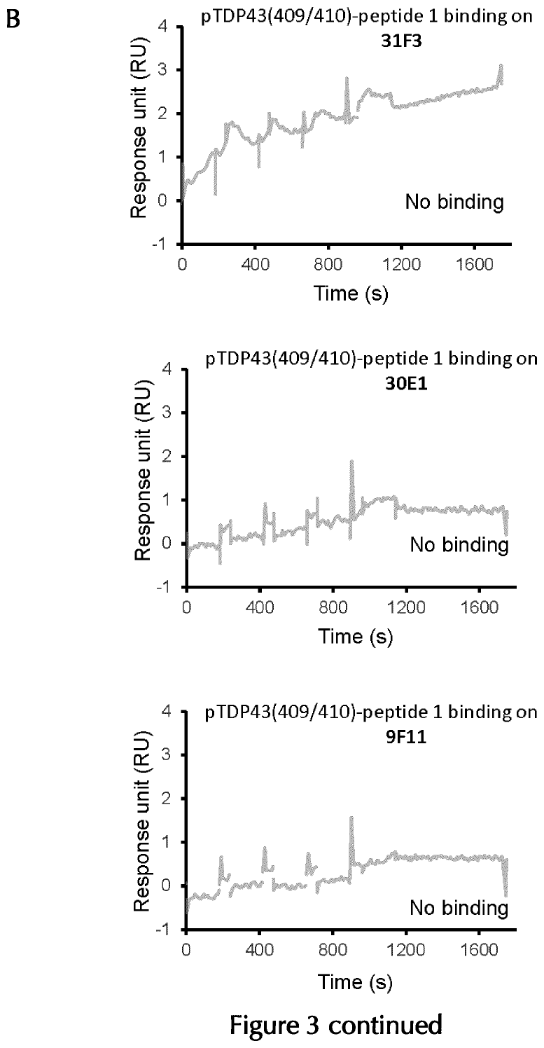

As shown in FIG. 3, specific binding was detected to pTDP-43 (403/404) (FIG. 3A), while no binding was found to pTDP-43 (409/410) (FIG. 3B), thereby confirming the results of Example 2. Table 4 below shows the binding parameters (ka, kd and KD) for binding to pTDP-43 (403/404).

TABLE 4
Binding parameters of exemplary antibodies
of the invention to pTDP-43 (403/404).

| Antibody | ka (1/Ms) | kd (1/s) | KD (nM) |
|---|---|---|---|
| 31F3 | 5.4e+5 | 5.1e−2 | 93 |
| 30E1 | 3.6e+5 | 2.5e−2 | 69 |
| 9F11 | 2.8e+5 | 6.0e−2 | 213 |

In summary, these data show that the antibodies provide high affinity and specificity to pTDP-43 (403/404).

### Example 4: Binding to High Molecular Weight Aggregates of TDP-43 and CTFs

Pathological inclusions contain full length TDP-43 and C-terminal fragments (CTF) of TDP-43, which are both phosphorylated. Most of the aggregates lack the N-terminal domain (NTD) of TDP-43. In view thereof, binding of antibody 31F3 to high molecular weight aggregates and C-terminal fragments of 25 kDa (CTF25) was investigated.

To this end, a western blot of brain samples from FTLD-TDP patients was performed using the 31F3 antibody. Briefly, homogenized brain tissue samples from selected patients were obtained. The samples were stored at −80° C. and transported on dry ice to avoid thawing of the tissue. For homogenization, the brain tissue was cut in a piece of approximately 300 mg with a sterile razor and placed into a 2-ml tube containing a mixture of ceramic beads with a diameter of 1.4 and 2.8 mm (Precellys, P000918LYSK0-A). 1× homogenization-solubilization buffer (HS buffer) (10 mM Tris-HCl pH 7.4, 150 mM NaCl, 0.5 mM EDTA, 1 mM Dithiotreitol, complete EDTA-free protease inhibitors (Roche), PhosSTOP phosphatase inhibitors (Roche)) was added in a ratio of 5:1 to the amount of tissue for a final concentration of 20%. The samples were then homogenized with a Minilys device (Bertin, P000673-MLYS0-A) or Precellys homogenizer (P000062-PEVO0-A) in three rounds for each 30 s, while cooling the samples on ice between the rounds. After homogenization, aliquots of 150 μL were produced in protein low-binding 1.5 ml tubes (Eppendorf, 0030108116). The aliquots were shock-frozen in dry ice and placed back into the −80° C. freezer.

SarkoSpin (Perez-Berlanga, M., Laferrière, F. and Polymenidou, M. (2019). SarkoSpin: A Technique for Biochemical Isolation and Characterization of Pathological TDP-43 Aggregates. Bio-protocol 9(22): e3424. doi: 10.21769/BioProtoc.3424) was performed on these samples. Briefly, 50 μL Benzonase mix, containing 14 mM MgCl2 and 250U benzonase (Merck Millipore, 71205-3) in 1×HS buffer was added to 150 μL of brain homogenate. After 5 minutes incubation at room temperature, 200 μL of 4% N-lauroyl-sarcosine (sarkosyl, Sigma, 61739) in 2×HS buffer (20 mM Tris-HCl pH 7.4, 300 mM NaCl, 1 mM EDTA, 2 mM Dithiotreitol, complete EDTA-free protease inhibitors (Roche), PhosSTOP phosphatase inhibitors (Roche)) was added to each sample. For solubilization, the samples were put on a heating block (Thermomixer, Eppendorf) for 45 minutes at 38° C. at 600 rpm. Afterwards, 200 μL of 0.5% sarkosyl in 1×HS buffer was added per sample and was centrifuged at 21200 g for 30 minutes at room temperature. The supernatant was discarded, and the pellet was cleaned twice with 100 μL phosphate buffered saline (Gibco, 10010015) to carefully remove lipids from the pellet. The pellet was then resuspended in 150 μL of 1×HS buffer.

For each sample, 20 μL were used and 7.5 μL of 4× Bolt™ LDS Sample Buffer (Life Technologies, B0007) and 3 μL of 10× Bolt™ Sample Reducing Agent (Life Technologies, B0009) were added. The samples were boiled at 90° C. for 10 minutes and loaded along with 5 μL of protein ladder (Thermofisher, 26616) in lanes (one sample/ladder per lane) of a Bolt 4-12% gel (Life Technologies, NW04125BOX). The ladder and samples were run for 10 min at 90 V and then for approximately 45 min at 130 V. The gel was transferred on a membrane (Life Technologies, 1B23001) using the iBlot2 gel transfer device (Life Technology, IB21001) with a 7 min transfer program at 20 V. The membrane was blocked for 1 h in 5% Milk (Coop, 7610800996958) in a PBS-Tween20 (PBS-T, PBS containing 0.025% Tween-20, Sigma, P1379) solution. The membrane was washed thrice with the PBS-T solution and the primary antibody phosphoTDP-43 (Mabylon, MY001-31F3 at 1:1000) was incubated over night at 4° C. in the PBS-T solution with 2.5% of BSA (Sigma Aldrich, A4503-100G). The membrane was washed thrice with PBS-T before incubation with secondary antibody Goat anti-Mouse HRP (Jackson Immuno Research, 115-035-146) for 1 h in PBS-T solution with 1% Milk. After washing thrice 10 min with PBS-T, images were taken with the Fusion FX imager (Vilber) using Femto substrate (SuperSignal™ West Femto Maximum Sensitivity Substrate, Life Technologies, 34095) following the manufacturer's instruction.

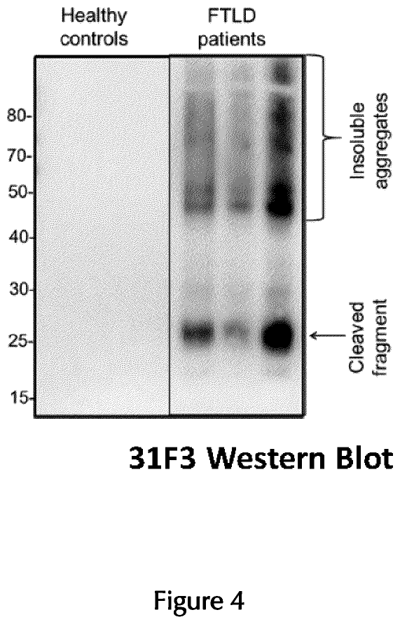

Results are shown in FIG. 4. The data show binding of antibody 31F3 to insoluble TDP-43 aggregates and CTF25 of FTLD patients, while no binding was detected for healthy controls. These data demonstrate the specificity of the antibody to pathological TDP-43.

### Example 5: Immunofluorescence in NSC-34 Cells Overexpressing EGFP-TDP-43

Next, immunofluorescence of motor-neuron-like NSC-34 cells overexpressing GFP-TDP-43 was studied. It is known that overexpression of TDP-43 leads to spontaneous cytoplasmic TDP-43 aggregates, therefore associated to TDP-43-related pathological phenotype. The inventors have previously shown that upon transient overexpression of human TDP-43, motor neuron-like hybrid cells (NSC-34, Cedarlane Laboratories, CLU140) develop cytoplasmic aggregates that become abnormally phosphorylated (Afroz et al. Functional and dynamic polymerization of the ALS-linked protein TDP-43 antagonizes its pathologic aggregation 8(1):45 (2017) doi: 10.1038/s41467-017-00062-0), reminiscent to the characteristic pathology found in sporadic ALS patient tissue.

To this end, 24-well plates (Ibidi, 82406) were pre-coated with Matrigel (Corning, 356255). Mouse motor neuron-like hybrid cells (NSC-34, Cedarlane Laboratories, CLU140) were plated at a density of 6*10⁴ cells per well and grown 48 h at 37° C. in an atmosphere of 5% CO2 in 500 μL of their appropriate culture medium (DMEM-F12 (Life Technologies, 21331-020) supplemented with 1× penicillin-streptomycin (Life Technologies, 15140-122), 1× GlutaMAX (Life Technologies, 35050-038), 1×N-2 Supplement (Life Technologies, 17502001), 2×B-27 Supplement (Life Technologies, 17504001), 1:1000 BDNF (Peprotech, 450-02-100UG) and 1:1000 GDNF (Alomone labs, G-240)). After that time, transfection (200 ng of DNA/well) for transient over-expression of GFP-TDP-43 (GFP-tagged wild type human TDP-43 plasmid, figure #5 from Afroz et al. Functional and dynamic polymerization of the ALS-linked protein TDP-43 antagonizes its pathologic aggregation 8(1):45 (2017) doi: 10.1038/s41467-017-00062-0 (which is incorporated herein by reference) was obtained with Lipofectamine 2000 (Life technologies, 11668-019) according to the manufacturer's instruction. After 24 h incubation with transfection reagents, the medium was replaced with normal growth medium. 48 h post transfection, medium was removed and cells were incubated 30 min with Matrigel at 37° C. Cells were successively washed with PBS (Life Technologies, 70011044), fixed for 10 min with 4% PFA, washed with quenching solution (100 mM Glycine (Sigma Aldrich, G7126-500G) in PBS) and stored in fresh PBS at 4° C.

For immunostaining, the cells were first permeabilized and blocked for 1 h at room temperature (RT) with 10% Donkey Serum (Sigma Aldrich, S30-100ML) and 0.1% Triton X-100 (Sigma Aldrich, T9284-500ML) in PBS. Primary antibodies (Mabylon, 31F3 at 1:250 and TDP-43 (Proteintech, 10782-2-AP) at 1:500) in PBS with 10% Donkey Serum, 0.1% Triton X-100 were added and incubated overnight at 4° C. Cells were washed thrice 5 min with PBS before incubating them with secondary antibodies (Donkey anti-Mouse Alexa 594 (Life Technologies, A-21203) and Donkey anti-Rabbit Alexa 647 (Life Technologies, A-31573) at 1:750) for 1 h at RT and subsequently washed thrice with PBS. DAPI (Life Technology, 62248, stock: 1 mg/ml, dilution 1:1000) staining was performed 5 min at RT. After washing thrice with PBS, coverslips were mounted with ProLong anti-fade medium with DAPI (Invitrogen, P36961) and the plate was kept for at least 24 h in the dark before imaging with a confocal microscope as described in Example 7 below.

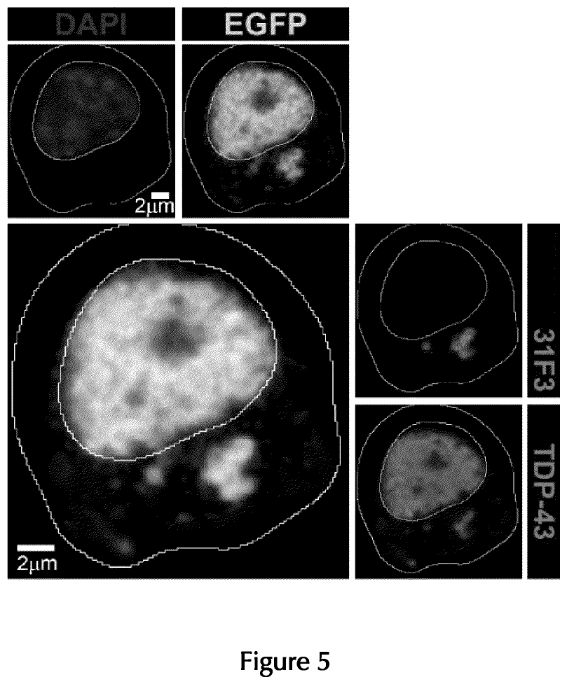

Results are shown in FIG. 5. Immunofluorescence of motor neuron-like NSC-34 cells overexpressing GFP-TDP-43 reveals co-localization of the 31F3 antibody with pathological cytoplasmic TDP-43 accumulations, but not with non-pathological nuclear TDP-43, again indicating the antibody's specificity for pathological TDP-43.

### Example 6: Immunofluorescence on Brain Sections of Patients with FTLD Pathology

Next, immunofluorescence of brain sections (frontal cortex) of FTLD patients with TDP-43 pathology was assessed.

To this end, 15 μm thick paraffin sections from fronto-parietal cortex of patients with FTLD pathology or non-neurodegenerative control were received from the bank tissue from University College of London. Sections were first deparaffinized by washing two times in xylene for 30 min at room temperature (RT), before being rehydrated by washing 2 times in 100% ethanol for 10 min at RT, 2 times in 95% ethanol for 5 min at RT, one time in 80% ethanol for 5 min at RT, one time in 70% ethanol for 5 min at RT, one time in 50% ethanol for 5 min at RT and one time in distilled water for 5 min at RT. Epitope retrieval was performed by incubating the section in citrate buffer-0.1% tween (pH 6.0) for 2 h at 80° C. After cooling down, sections were incubated in an autofluorescence quenching buffer (20 mM Glycine in PBS-0.1% triton) for 1 h at RT and then in the blocking buffer (3% BSA, 10% donkey serum in PBS-0.25% triton) for 1 h at RT. Antibody 31F3 was incubated (stock: 1.47 mg/mL, 1:500) in the blocking buffer for 48 h at 4° C. After three PBS washes, the secondary antibody was incubated in the blocking buffer for 48 h at 4° C. After three PBS washes, sections were post-fixed with cold 4% PFA-4% sucrose solution for 5 min followed by three PBS washes. For super-resolution microscopy (STED), no DAPI staining was performed. For confocal imaging, sections were incubated in DAPI solution (Life Technology 62248, stock: 1 mg/ml, dilution 1:1000) for 30 min before being washed three times in PBS. Sections were then mounted using prolong diamond anti-fade mounting medium (Invitrogen, P36961) and covered using Carl Zeiss glass coverslips high performance compatible with super resolution microscopy (Carl Zeiss, 10474379).

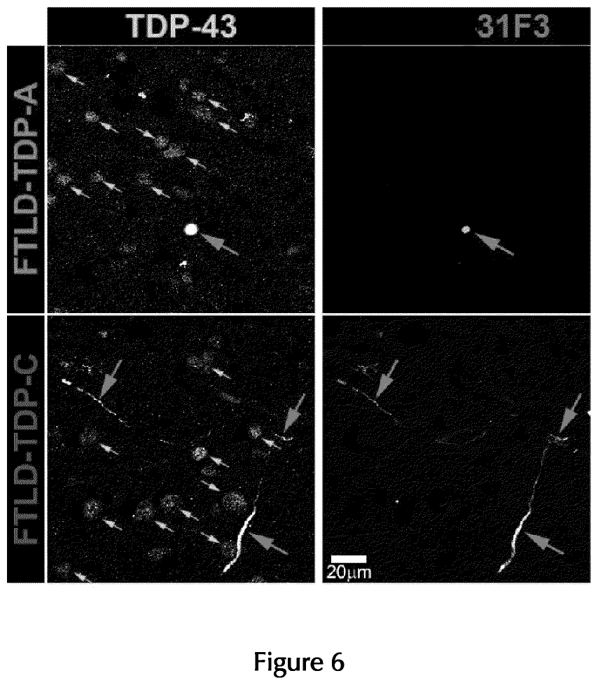

Results are shown in FIG. 6. Immunofluorescence of brain sections (frontal cortex) of FTLD patients with TDP-43 pathology shows co-localization of the 31F3 antibody with neuronal cytoplasmic inclusions and dystrophic neurites. Nuclear TDP-43 in neighboring non-affected cells is not detected by 31F3.

### Example 7: Functional Testing of Antibodies in HEK293 Seeding Model

A crucial hurdle in the development of therapeutics against TDP-43 proteinopathies is the lack of experimental models faithfully recreating the pathological features seen in human patients. Recently, a new cellular model with TDP-43 pathology was developed by employing a protein seeding paradigm in cells without strong overexpression of TDP-43 (Laferrière, F. et al. TDP-43 extracted from frontotemporal lobar degeneration subject brains displays distinct aggregate assemblies and neurotoxic effects reflecting disease progression rates. Nat. Neurosci. 22, 65-77 (2018)). By inoculating cells that inducibly express physiological levels of traceable TDP-43 (TDP-43-HA) with patient derived extracts, disease-like cytoplasmic TDP-43 aggregates with concomitant nuclear clearance can be generated. Importantly, this model allows to test different types of aggregates extracted from different subtypes of FTLD-TDP or ALS. Patient-derived pathological TDP-43 assemblies function as templates for aggregation of TDP-43-HA, leading to faithful aggregates that recapitulate different disease subtypes.

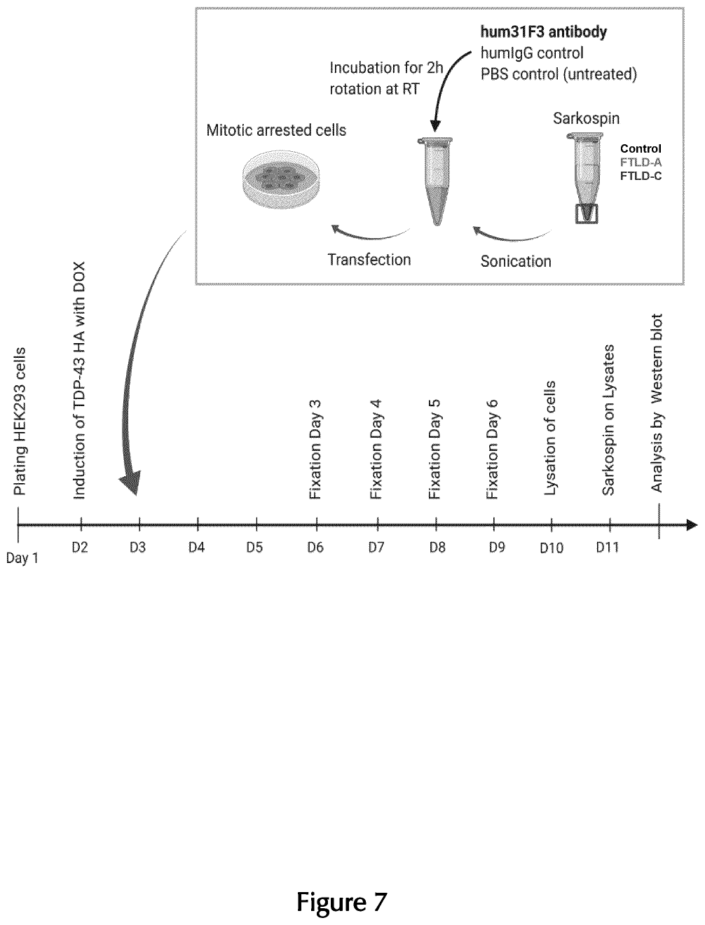

An overview over the workflow of antibody testing in this new model (HEK293 seeding model) is provided in FIG. 7.

This model is based on stable HEK293 cell lines inducibly expressing tagged TDP-43, using the Flp-In™ T-REx™ technology from Invitrogen (Hauri, S. et al. A High-Density Map for Navigating the Human Polycomb Complexome. Cell Rep. 17, 583-595 (2016)). Expression of TDP-43-HA fusion protein allows distinction of newly induced from exogenous aggregates. It was previously shown that inoculation of these TDP-43-HA-expressing cells with patient-extracted material leads to aggregation of TDP-43-HA accompanied by reduced cell viability (Laferrière, F. et al. TDP-43 extracted from frontotemporal lobar degeneration subject brains displays distinct aggregate assemblies and neurotoxic effects reflecting disease progression rates. Nat. Neurosci. 22, 65-77 (2018)).

To confirm that exogenous FTLD-TDP-A and FTLD-TDP-C aggregates induced aggregation of TDP-43-HA in the HEK293 seeding model, brain homogenates of FTLD-TDP-A and FTLD-TDP-C patients were used. Brain homogenates were obtained as described above in Example 4.

SarkoSpin on brain homogenates

50 μL Benzonase mix, containing 14 mM MgCl2 and 250U benzonase (Merck Millipore, 71205-3) in 1×HS buffer was added to 150 μL of brain homogenate. After 5 minutes incubation at room temperature, 200 μL of 4% N-lauroyl-sarcosine (sarkosyl, Sigma, 61739) in 2×HS buffer (20 mM Tris-HCl pH 7.4, 300 mM NaCl, 1 mM EDTA, 2 mM Dithiotreitol, complete EDTA-free protease inhibitors (Roche), PhosSTOP phosphatase inhibitors (Roche)) was added to each sample. For solubilization, the samples were put on a heating block (Thermomixer, Eppendorf) for 45 minutes at 38° C. at 600 rpm. Afterwards, 200 μL of 0.5% sarkosyl in 1×HS buffer was added per sample and was centrifuged at 21200 g for 30 minutes at room temperature. The supernatant was discarded, and the pellet was cleaned twice with 100 μL phosphate buffered saline (Gibco, 10010015) to carefully remove lipids from the pellet. The pellet was then resuspended in 200 μL Phosphate buffered saline (PBS, Gibco 10010015) for seeding on HEK cells by sonication (Qsonica, Q2000) with an amplitude of 60% power and 3 seconds on/3 seconds off for 3 minutes. The sample was used fresh to seed on cells.

Seeding with SarkoSpin Pellets on HEK Cells

HEK293T Flp-In-T-REx (Invitrogen) cells expressing TDP-43-HA under doxycycline (dox, Clontech, 631311) induction were generated as described in Laferrière et al. 2019. HEK293T cells were cultured in Dulbecco's Modified Eagle's medium (DMEM, Sigma D5671), supplemented with 10% fetal calf serum (FCS, Life Technologies A3160802), 1×GlutaMAX (Gibco 35050038), 1× penicillin-streptomycin (Sigma P4333), 0.2% hygromycin (Invitrogen 10687010) and 0.04% blasticidin (Invitrogen 4069-ant-bl-10p), and maintained in incubators at 37° C. and 5% CO₂. Plates were coated with 100 μg/ml Poly-D-Lysine Hydrobromide (PDL) and incubated at 37° C. for at least 1 h. HEK293 cells were plated at 225 cells/mm². After one day, TDP-43-HA was induced by adding dox in a ratio of 1:500 in the HEK stable medium. One day later, the SarkoSpin protocol was performed. The SarkoSpin pellet was prepared for seeding by incubating it with OptiMEM and Lipofectamine2000 (ratio 1:2.5˜SarkoSpin pellet to Lipofectamine) for 30 minutes. 0.05 μg sarkospin pellet was added for 1 mm² coverslip. After incubation of 3-4 h, HEK media containing 20 nM AraC and dox (1:500) was added. One day after seeding, the media was changed with HEK media containing 20 nM AraC and dox (1:500).

Fixation of Seeded HEK Cells, Staining and Mounting of Samples

At specific time points (day 3-day 6), the wells were first washed once with PBS and then fixed for 30 min with a 4% PFA and 4% sucrose solution. After fixation, the plates were again washed once with PBS and stored in fresh PBS at 4° C. PBS was removed from the wells and a 50 mM NH₄Cl in PBS with 0.25% Triton solution was incubated for 1 h, to quench autofluorescence. After removing of the quenching solution, the primary antibody in saturation buffer (10% donkey serum, 3% BSA and 0.25% Triton in PBS) was added and incubated overnight at 4° C. The next day, the plates were put at room temperature for 1 h to let them warm up gently. Then, the wells were washed three times with PBS before the secondary antibody in saturation buffer was added. After incubation for 2-3 h in the dark at room temperature, the plates were washed three times with PBS. The staining was stabilized by adding 4% PFA, 4% sucrose in PBS solution for 5 min. After another three washes with PBS, DAPI was stained for 30 min in the dark at room temperature. The plates were washed another three times and then placed back in the 4° C. fridge. Coverslips were mounted with Prolong Diamond Antifade Mountant (Invitrogen, P36961). TDP-43-HA (C29F4, Cell Signaling) was stained coupled with secondary antibody alexa 568, phosphorylated TDP-43 (MY001-31F3, Mabylon) coupled with secondary antibody alexa 647 and LaminB1 (66095-1-Ig, Proteintech) coupled with secondary antibody alexa 488 for confocal and dSTORM imaging. All secondary antibodies were purchased from Thermo Fisher Scientific.

Harvesting Seeded HEK Cells for Biochemical Analysis

At specific time points (day 3-day 6), the dishes were harvested for western blot analysis. Media was removed and the plate was washed once with PBS. After PBS is removed, the plate was shock-frozen on dry ice and then stored in the −80° C. freezer. To prepare the sample for western blot analysis, the plate was quickly thawed on ice. The cells were scraped first in 200 μL 0.5% sarkosyl 1×HS buffer containing 25 U benzonase and 1.4 mM MgCl2 and afterwards in another 200 μL 0.5% sarkosyl 1×HS buffer without benzonase. In order to determine protein concentration, a BCA protein assay was performed according to the manufacturer's instructions (Pierce, 23227). For further steps, the same amount of protein concentration was taken for all samples. For solubilization, the appropriate amount of 4% sarkosyl in 2×HS buffer was added, to reach a final concentration of 2% sarkosyl. The samples were incubated on ice for 30 min, while vortexing every 10 min. 50 μL of total lysate was stored for further analysis. The rest of the sample was centrifuged at 15′000 g for 30 min at room temperature. After centrifugation, 200 μL of supernatant was put aside for further analysis. The rest of the supernatant was disposed, and the pellet was resuspended in 50 μL 0.5% sarkosyl in 1×HS buffer. All samples were stored either at −20° C. for short-term or −80° C. for long-term storage.

Western Blot on Seeded Cell Lysates

The samples were prepared for western blot by adding 1× Laemmli buffer (4% SDS (Bisolve, 19822359), 20% Glycerol (Sigma, G7757), 0.004% Bromophenol blue (Sigma, B5525), 0.125 M Tris-HCl pH8 (Sigma, T3253), 10% 2-mercaptoethanol (Sigma, M3148)) and denaturation for 10-15 min at 95° C. Samples were loaded along with a protein ladder (Thermofisher, 26616) on a 4-12% Bis-Tris gel (Life Technology, NW04125BOX) and were run or 10 min at 80 V and then for approximately 45 min at 120 V. The gel was transferred on a PVDF membrane (Life Technology, IB24001) using the iBlot2 gel transfer device (Life Technology, IB21001) with a 7 min transfer program at 20 V. The membrane was blocked for 1 h in a 1% bovine serum albumin (BSA, Sigma, A4503), 1% cold fish gelatin (Sigma, G7765) in PBS-Tween20 (PBS-T, PBS containing 0.025% Tween-20, Sigma, P1379) blocking solution. Primary antibody against HA (C29F4, Cell Signaling, 1:1000) was incubated over night at 4° C. in the blocking solution. The membrane was washed three times with PBS-T before incubation with secondary antibody alexa 568 (A1 0037, Thermo Fisher Scientific) for 1-2 h at room temperature in the dark. After three 10 min washes with PBS-T, images were taken with the Fusion FX imager (Vilber).

Microscopy and Image Processing

Confocal images were taken with Leica Falcon SP8 at the Center for Microscopy and Image Analysis (ZMB) at the University of Zurich. Images were acquired using HC PL APO corr CS2 20× objective (NA 0.75) at a 2048×2048 pixels resolution achieving 227 nm pixel resolution. For the high magnitude images, the SP8 STED 3× with no STED activated was used using HC PL APO STED WHITE 100× objective (NA 1.4). Microscope parameters were kept constant between conditions and experiments. 3 independent experiments were imaged with 7 control, 7 FTLD-TDP-A and 6 FTLD-TDP-C cases. The images were deconvoluted with Huygens and processed with Imaris Version 9.3 by reconstructing the image using surfaces. Surface parameters were created by choosing an intensity and size threshold for DAPI, nuclear TDP-43-HA, aggregated TDP-43-HA and phosphorylated TDP-43. For TDP-43-HA, the same intensity threshold but different size parameters were applied to distinguish between nuclear (volume above 235 μm³) and aggregated TDP-43-HA (area between 1.25 μm² and 234 μm²). The same size parameter used for aggregated TDP-43-HA (area between 1.25 μm² and 234 μm²) was applied to phosphorylated TDP-43. Number of voxels was fixed to be above 10.0 for all surfaces, except for the surface of DAPI where it was fixed at 2500. Grain size of the surface for DAPI was fixed at 0.5 μm, for nuclear TDP-43-HA at 0.4 μm and for aggregated TDP-43-HA and phosphorylated TDP-43 at 0.2 μm. Surface to surface co-localization between DAPI and nuclear TDP-43-HA and between aggregated TDP-43-HA and phosphorylated TDP-43 was determined. Co-localization surface of the surfaces DAPI and nuclear TDP-43-HA was smoothed with a grain size of 0.454 whereas the co-localization surface of the surfaces aggregated TDP-43-HA and phosphorylated TDP-43 was not smoothed. To visualize mask images and for distance calculations, Imaris Version 9.6 was used.

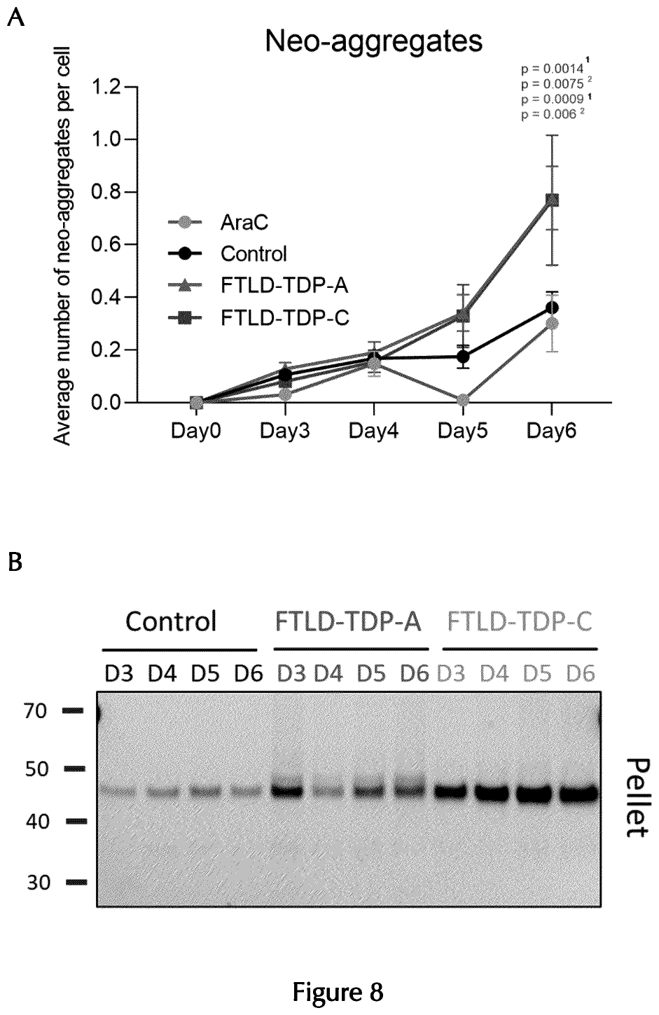

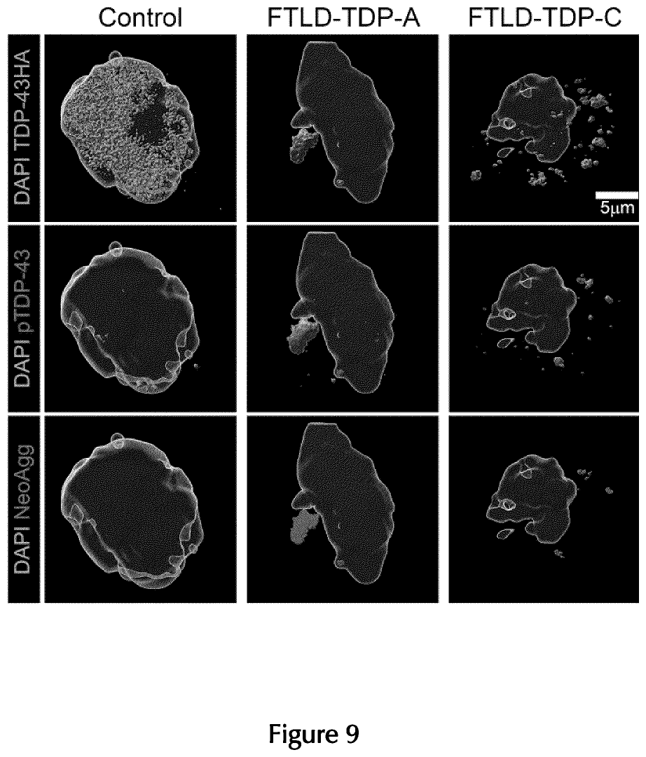

Results are shown in FIGS. 8 and 9. FIG. 8A shows the quantification of neo-aggregates (NeoAgg; defined as phosphorylated, cytoplasmic HA-tagged TDP-43 (TDP-43-HA)) overtime, showing their increase upon incubation with FTLD-TDP-A and FTLD-TDP-C, compared to non-degenerative brain sample and a non-transfected control. FIG. 8B shows the results for the western blot, which show an increase of sarkosyl-insoluble TDP-43-HA aggregates (pellet) in seeded HEK293 cells with brain material from FTLD-TDP-A and FTLD-TDP-C, compared to non-degenerative brain sample control. The insoluble TDP-43-HA is increased overtime.

FIG. 9 shows that that neo-aggregates (NeoAgg) of HEK293 cells transfected with FTLD-TDP-A or FTLD-TDP-C co-localize with antibody 31F3 detecting pTDP-43. Moreover, presence of large cytoplasmic NeoAgg in HEK293 cells seeded with FTLD-TDP-A and multiple, smaller cytoplasmic NeoAggs seeded with FTLD-TDP-C are revealed.

Accordingly, the data confirm that exogenous FTLD-TDP-A and FTLD-TDP-C aggregates induced aggregation of TDP-43-HA, consistent with seeding potential of the exogenous patient-derived aggregates. These neo-aggregates become phosphorylated and are specifically identified by the 31F3 antibody (FIG. 9). They increase over time (FIG. 8A,B), similar to the situation observed in patient brains and spinal cords. The data also suggest that the kinetics of triggered aggregation as well as the size of neo-aggregates are different in FTLD types A and C, suggesting that diverse disease characteristics are captured with this cell culture system.

In view thereof, this cellular model of TDP-43 pathology is used to test the antibody's ability to block or reduce the formation of pathological TDP-43 neo-aggregates. To this end, TDP-43-HA expressing HEK cells are seeded with pathological aggregates extracted by SarkoSpin from post-mortem FTLD brain samples as described above with or without pre-incubation with antibody 31F3. An overview over the workflow is provided in FIG. 7.

Briefly, The SarkoSpin pellet was prepared as described above and incubated with 31F3 human IgG (stock concentration 1.47 mg/ml) or IgG control (stock concentration: 1 g/L) on rotation for 2 h at room temperature in a ratio of 1:5 (SarkoSpin pellet˜antibody). An untreated sample was kept on ice during the incubation time. After incubation, the treated and untreated SarkoSpin pellet was transfected on HEK cells, fixed at day 3 to day 6 and a western blot analysis was done, following the normal protocol described above.

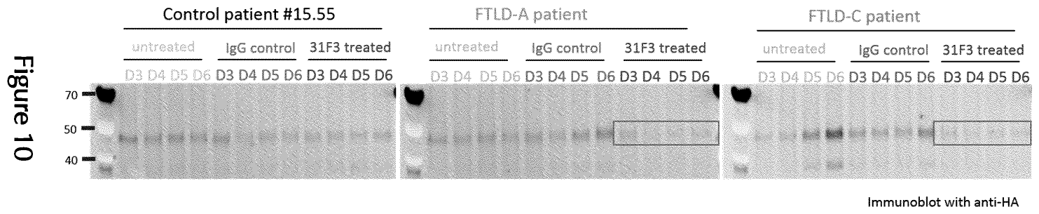

Results are shown in FIG. 10. These data show that antibody 31F3 decreases TDP-43 aggregates in the HEK cell seeding model in both, FTLD-A and FTLD-C samples. This demonstrates the protective effect of the antibody on TDP-43 pathology in both, FTLD-A and FTLD-C.

### Example 8: Immunohistochemistry in CNS Sections of Patients with Distinct Diseases Involving TDP-43 Proteinopathy

To test the reactivity of the antibody in different diseases with TDP-43 proteinopathy, CNS sections from patients suffering from TDP-43-related diseases were studied with 31F3 immunohistochemistry.

To this end, (i) thoracic spinal cord from 75 year old female diagnosed with ALS 4 years prior to death, (ii) subiculum of a 58 year old male with incidental finding of Alzheimer's disease pathology with granulovacuolar degeneration (GVD); and (iii) subiculum of a 91 year old male with clinical suspicion of Alzheimer's disease vs. vascular dementia/diagnosis at autopsy: limbic-predominant age-related TDP-43 encephalopathy (LATE) was studied.

Briefly, 2 μm sections from formalin-fixed, paraffin embedded samples were processed on a Ventana BenchMark Ultra automated slide stainer (Roche). Epitope retrieval was performed using built-in program CC1 16 at 100° C. 31F3 was added at a dilution of 1:2′400 for 30 min at room temperature. OptiView DAB IHC Detection Kit (Roche) was used for secondary anti-mouse antibody and immunohistochemical development according to the manufacturer's guidelines.

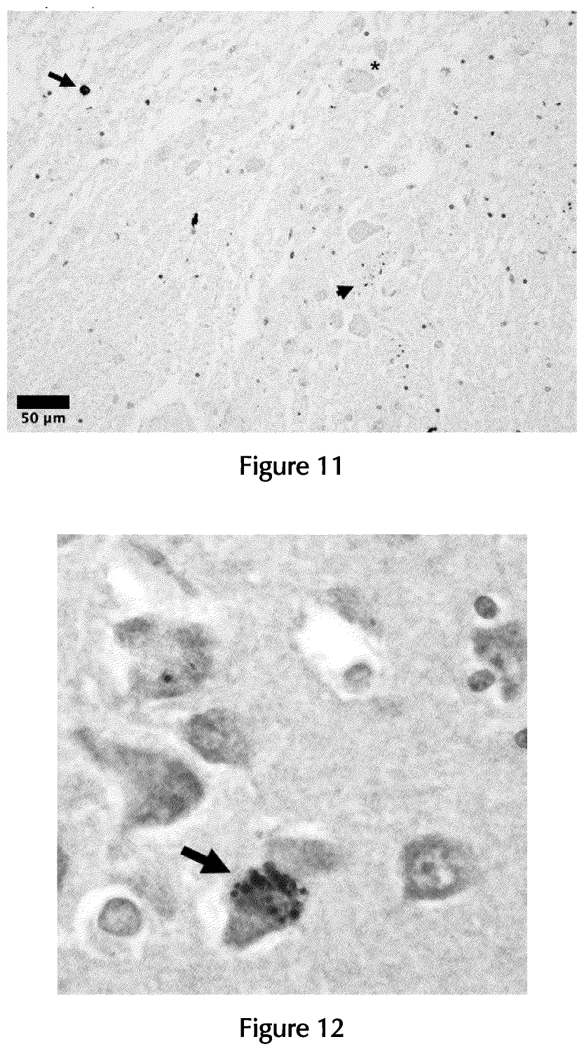

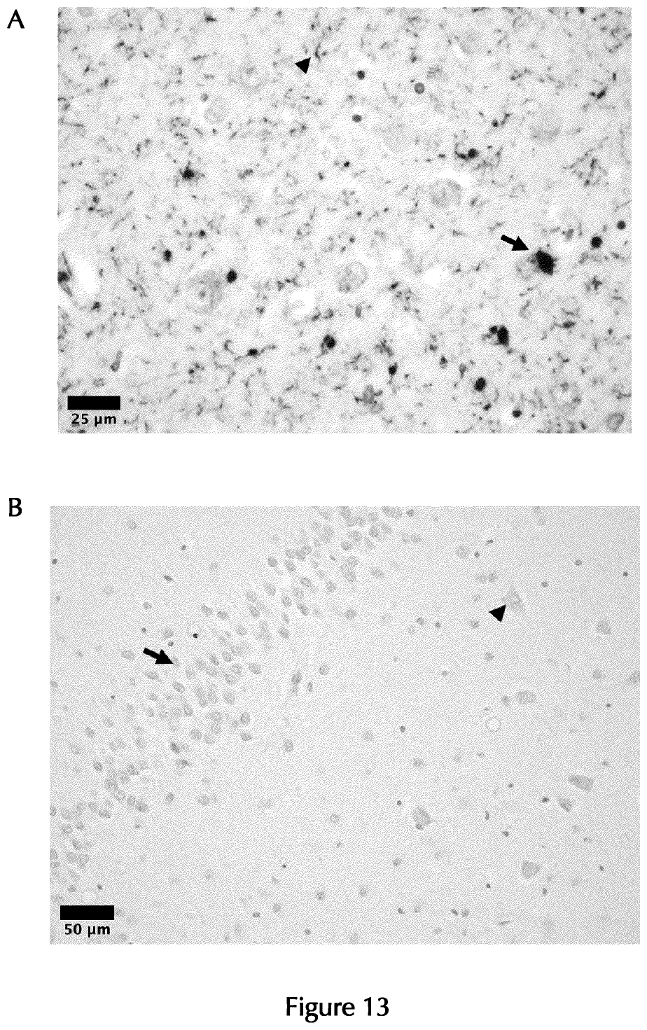

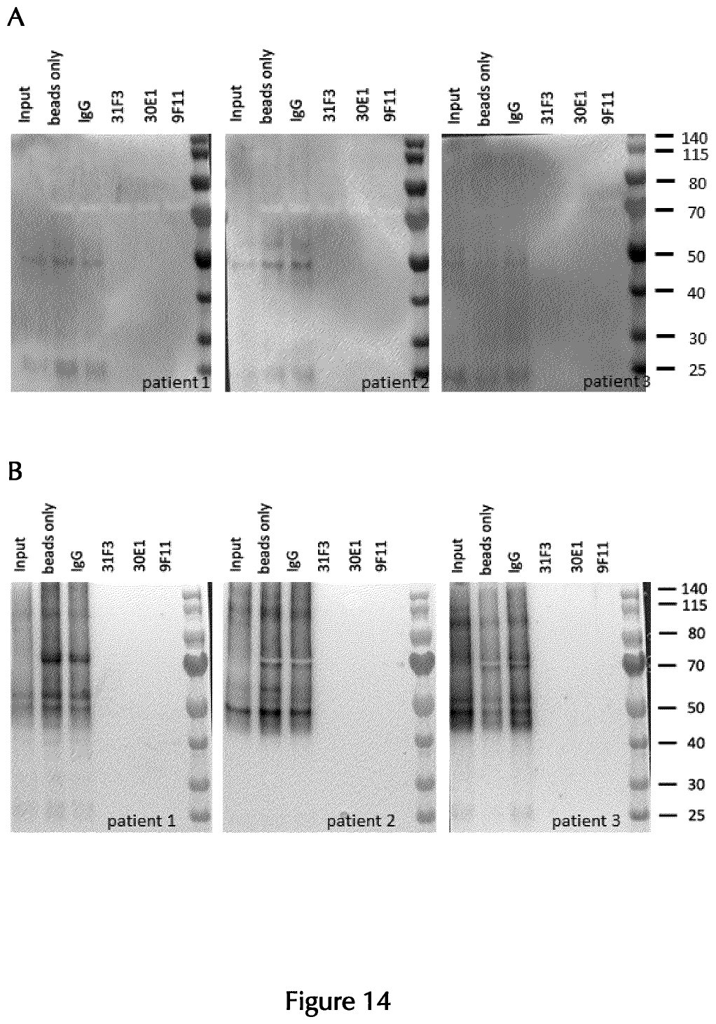

Results are shown in FIGS. 11 to 13. In addition to the FTLD-results reported above, antibody 31F3 also showed specific detection of TDP-43 inclusions in ALS (FIG. 11), in Alzheimer's disease pathology with Granulovacuolar degeneration (FIG. 12), and in limbic predominant age-related TDP-43 encephalopathy (LATE, FIG. 13). This indicates the universality of the antibody for different diseases with TDP-43 proteinopathy.

### Example 9: SPR Binding Study to Full-Length TDP-43

To test binding of the antibodies to full-length TDP-43, full-length TDP-43 (SEQ ID NO: 1) was phosphorylated in vitro as recently described in da Silva et al. 2022 (Gruijs da Silva L A, Simonetti F, Hutten S, Riemenschneider H, Sternburg E L, Pietrek L M, Gebel J, Dötsch V, Edbauer D, Hummer G, Stelzl L S, Dormann D. Disease-linked TDP-43 hyperphosphorylation suppresses TDP-43 condensation and aggregation. EMBO J. 2022 Feb. 3:e108443. doi: 10.15252/embj.2021108443. Epub ahead of print. PMID: 35112738). In brief, recombinant TDP-43-MBP (maltose binding protein) was thawed on ice, cleared 15 min at 17000 g at 4° C., transferred to a new protein loBind tube (Eppendorf), and incubated 2 h at room temperature in the presence of 200 μM ATP (Sigma, cat #A9187) and a 2-fold molar excess of Casein kinase 18 (Millipore, cat #14-520M) diluted in freshly prepared phosphorylation buffer (50 mMTris-HCl pH7.5, 10 mM MgCl2, 1 mM DTT). Samples were stored at −20° C. until analysis. As shown in da Silva et al. 2022, this results in phosphorylation of TDP-43 at multiple sites.

Binding properties (ka, kd and KD) of selected antibodies with respect to recombinant in vitro phosphorylated TDP-43 (pTDP-43) were determined at 25° C. using a real-time biosensor surface plasmon resonance (SPR) assay (BIACORE™ 8K), using 10 mM HEPES pH 7.4, 150 mM NaCl, 3 mM EDTA and 0.005% Tween-20 as running buffer. In vitro phosphorylated TDP-43 was immobilized on the surface of CM5 sensor chip 250 nM concentration through standard amine coupling. Increasing concentrations (0.3, 0.6, 1.2, 2.5, 5, 10 nM of 30E1 or 31F3 antibodies as a Fab or full IgG were injected over the captured antibody surface at 30 μl/min for 180 sec to measure association rates (ka). Dissociation rate (kd) was monitored for 600 sec. Analyte responses were corrected for unspecific binding and buffer responses. Curve fitting and data analysis to determine the kinetic parameters, were performed with Biacore™ Insight Evaluation Software v2.0.15.12933.4

Table 5 below shows the binding parameters (ka, kd and KD) for binding to full-length pTDP-43.

TABLE 5
Binding parameters of exemplary antibodies of
the invention as Fab or full IgG to pTDP-43.

| Antibody | ka (1/Ms) | Kd (1/s) | KD (nM) |
|---|---|---|---|
| 31F3 (IgG) | 2.5e+7 | 3.0e−4 | 0.01 |
| 31F3 (fab) | 6.8e+6 | 1.7e−2 | 2.5 |
| 30E1 (IgG) | 3.0e+7 | 6.1e−4 | 0.02 |
| 30E1 (fab) | 3.1e+6 | 1.1e−1 | 34 |

In summary, these data show that the antibodies provide high affinity and specificity also to full-length pTDP-43 in different formats (Fab or IgG). The Fab data show the affinity of the antibody against the target, while the IgG data represent the avidity of the antibodies against the target. Moreover, the data also suggest that binding of pTDP-43 is not affected by phosphorylation at other sites.

### Example 10: Immunodepletion of FTLD Brain Samples

To solubilize brain tissue samples, brain tissue samples from FTLD patients were stored at −80° C. and transported on dry ice to avoid thawing of the tissue. For homogenization, the brain tissue was cut in a piece of approximately 300 mg with a sterile razor and placed into a 2-ml tube containing a mixture of ceramic beads with a diameter of 1.4 and 2.8 mm (Precellys, P000918LYSK0-A). 1× homogenization-solubilization buffer (HS buffer) (10 mM Tris-HCl pH 7.4, 150 mM NaCl, 0.5 mM EDTA, 1 mM Dithiotreitol, complete EDTA-free protease inhibitors (Roche), PhosSTOP phosphatase inhibitors (Roche)) was added in a ratio of 5:1 to the amount of tissue for a final concentration of 20%. The samples were then homogenized with a Minilys device (Bertin, P000673-MLYS0-A) or Precellys homogenizer (P000062-PEVO0-A) in three rounds for each 30 s, while cooling the samples on ice between the rounds. After homogenization, aliquots of 150 μL were produced in protein low-binding 1.5 ml tubes (Eppendorf, 0030108116). The aliquots were shock-frozen in dry ice and placed back into the −80° C. freezer.

For immunodepletion, 20 μg of human 31F3, 30E1, 9F11, or human IgG antibodies were conjugated to 50 μl of protein G beads (Invitrogen 10004D) on rotation for 1 h at 4° C. Beads were washed 3× in PBS 1% BSA and blocked 30 min on rotation in PBS 10% BSA. Aggregates from 150 μl homogenized FTLD-TDP brain sections were solubilized in 200 μl of 4% sarkosyl in 2×HS buffer using a heating block (ThermoMixer) for 45 min at 37° C. and 600 rpm (SarkoSpin method). 0.5% sarkosyl in 1×HS buffer was added per sample, giving a total solubilized sample of 550 μl which was used for immunodepletion. Beads were resuspended at a concentration of 500 nM in 180 μL solubilized brain tissue samples and incubated on rotation at 4° C. for 3 hours. Beads and supernatant were separated as per manufacturer instructions. Beads were resuspended in PBS and the post-immunoprecipitation supernatant was used to enrich the remaining aggregates by continuing the SarkoSpin method. In brief, the samples were centrifuged at 21200 g for 30 min at room temperature. The supernatant was discarded and the pellet was cleaned twice with 100 μl PBS to remove lipids and remaining supernatant. The pellet was then resuspended in 50 μl PBS by sonication (3 min, 2 sec on/2 sec off, 60% amplitude). To each 50 μl resuspended pellet, 19.2 μl of 4× Bolt™ LDS Sample Buffer (Life Technologies, B0007) and 7.7 μL of 10× Bolt™ Sample Reducing Agent (Life Technologies, B0009) were added. The samples were boiled at 80° C. for 10 min and 20 μl of sample was loaded on a 4-12% gel (Life Technologies, NW04125BOX), run for 10 min at 90 V and then for approximately 45 min at 130 V. The gel was transferred on a membrane (Life Technologies, IB23001) using the iBlot2 gel transfer device (Life Technology, IB21001) with a 7 min transfer program at 20 V. The membrane was blocked for 1 h in 5% milk (Coop, 7610800996958) in a PBS-Tween20 (PBS-T, PBS containing 0.025% Tween-20, Sigma, P1379) solution. The membrane was washed 3× with PBS-T and incubated with primary antibodies overnight at 4° C. in the PBS-T solution with 2.5% of BSA (Sigma Aldrich, A4503-100G). Primary antibodies were: pTDP-43 403/404 (Mabylon, murinized 31F3) total TDP-43 (3H8, Novus Bio) and pTDP43 409/410 (CosmoBio, CAC-TIP-PTD-M01). The membrane was washed 3× with PBS-T before incubation with secondary antibody Goat anti-Mouse HRP (Jackson Immuno Research, 115-035-146) for 1 h in PBS-T solution with 2.5% of BSA. After washing 3× for 10 min with PBS-T, images were taken with the Fusion FX imager (Vilber) using Pico substrate (SuperSignal™ West Pico PLUS Chemiluminescent Substrate, Life Technologies, 34577) following the manufacturer's instruction.

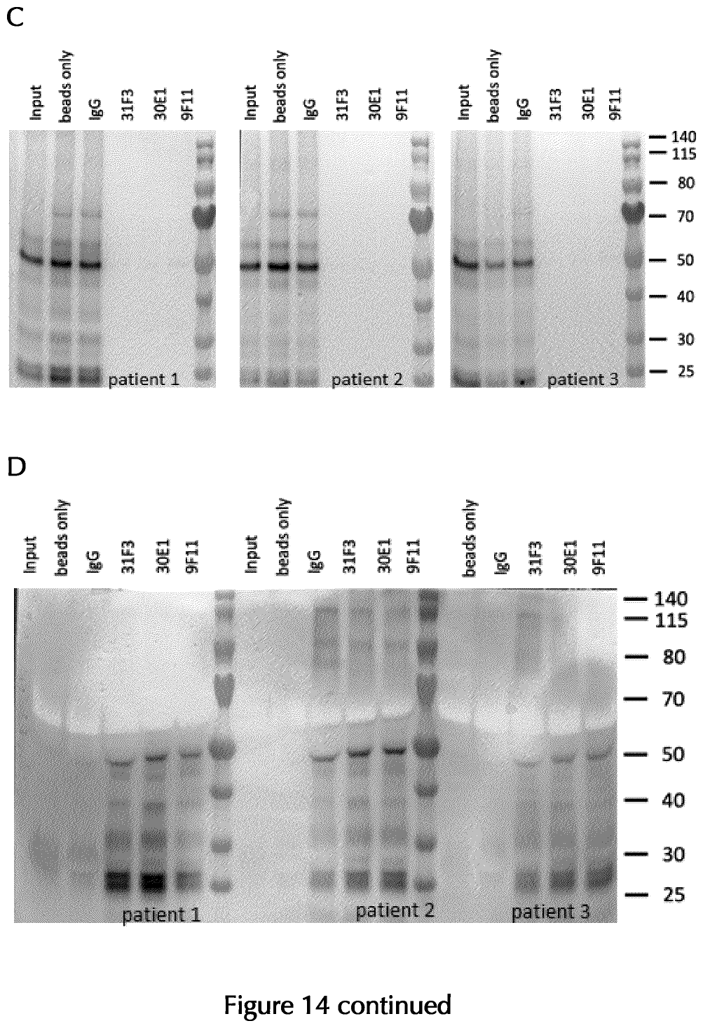

Results are shown in FIG. 14. The data demonstrate successful depletion of pTDP (S403/404) (FIG. 14 A) as well as depletion of total TDP-43 (FIG. 14 B) and pTDP-43 (S409/410) (FIG. 14 C). This shows that the antibodies are able to deplete all TDP-43 aggregated species from patient brain material. This indicates that the antibody can eliminate all species of TDP-43 aggregates from the brain of patients with TDP-43 pathology. In accordance with the seeding effect of the FTLD patient-derived sarkosyl-insoluble TDP43 shown in example 7, it is expected that depletion of the TDP-43 aggregates will prevent spreading and progression of TDP-43 pathology in patients with TDP-43 proteinopathies.

### Example 11: Concomitant Binding of Antibodies of the Invention and p409/410 Antibodies to Phosphorylated TDP-43

To test the ability of antibodies 31F3 and 30E1 to bind pS403/404 (TDP-43) when also S409/410 is phosphorylated, 0.5 ug/ml of 31F3 or 30E1 (as IgG antibodies) in PBS were captured with an anti-human IgG (AHQ) Biosensors (Fc-specific) on an OctetRED96e (Forte Bio, Sartorius) for 120 s (step 1). The sensors coupled to antibodies 31F3 and 30E1 were incubated with 1000 nM pS403/404 pS409/410 TDP-43 “peptide 4” (SEQ ID NO: 2, which corresponds to amino acids from position 391 to 414 of full-length human TDP-43 of SEQ ID NO: 1; phosphorylated at serine residues at positions 14, 15, 20 and 21 which correspond to the serine residues at positions 403, 404, 409 and 410 of SEQ ID NO: 1) 300 s (step 2). Subsequently the sensors were exposed to 5 ug/ml of a commercially available polyclonal anti pS409/410 TDP-43 antibody (proteintech, 66318-1-Ig) to detect concomitant binding of 31F3 and 30E1 with pS409/410 TDP-43 antibodies (step 3). As a control, no pS409/410 TDP-43 antibody binding was observed when using “peptide 2” (SEQ ID NO: 2, which corresponds to amino acids from position 391 to 414 of full-length human TDP-43 of SEQ ID NO: 1; phosphorylated at serine residues at positions 14 and 15, which correspond to the serine residues at positions 403 and 404 of SEQ ID NO: 1) in the second step, or when 30E1 was used in step 3.

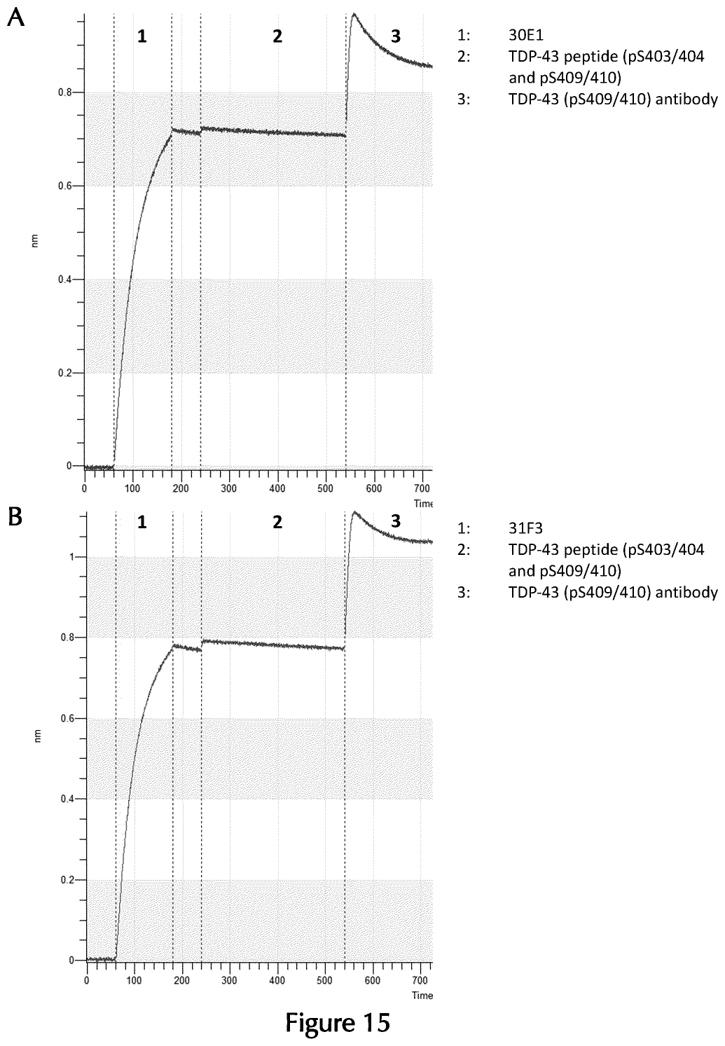

The results are shown in FIG. 15 for antibodies 30E1 (FIG. 15A) and 31F3 (FIG. 15B). These data demonstrate that (1) 31F3 and 30E1 can bind to the pS403/404 when another site is phosphorylated pS409/410; and (2) 31F3 and 30E1 can bind pS403/404 when another antibody, namely, polyclonal anti pS409/410 TDP-43 antibody (proteintech, 66318-1-Ig), is bound to pS409/410.

### Example 12: Framework Mutations do not Antibody Impair Binding to TDP-43

To investigate the effect of framework mutations, somatic framework mutations in antibody 31F3 were reverted to their residues from the germline sequences IGHV1-2*06 for the heavy chain and IGLV2-23*01 for the light chain. Two constructs for the heavy chain, Hf (VH: SEQ ID NO: 111) and Hg (VH: SEQ ID NO: 113), and two constructs for the light chain, Lf (VL: SEQ: 112) and Lg (VL: SEQ ID NO: 114), were designed. Different combinations of the engineered 31F3 variable regions (HgLg, HgLf, HfLg and HfLf) were expressed and purified as Fab antibodies and compared to their non-engineered counterpart.

Binding of the antibodies to the phosphorylated TDP-43 peptide as described in Example 1 (“peptide 2”: pS403/404 TDP-43) was tested by ELISA. 96 well microplates (Costar®, Corning Incorporated, Corning, NY, USA) were coated with 0.5 μg/ml pS403/404 TDP-43. Plates were washed with PBS-Tween 0.05% and blocked 1 h at room temperature with PBS containing 2% bovine serum albumin (BSA, Sigma-Aldrich Chemie GmbH, Buchs, Switzerland). Antibody preparations were incubated for 2 h at room temperature. Binding of human IgG or murine IgG or rabbit IgG to the antigen of interest was determined using a HRP-conjugated anti human antibody (anti-IgG-HRP from Jackson ImmunoResearch, West Grove, PA, USA) or HRP-conjugated anti-mouse antibody (goat anti-mouse IgG-HRP, Jackson ImmunoResearch, West Grove, PA, USA) or HRP-conjugated anti-rabbit antibody (goat anti-rabbit IgG-HRP, Jackson ImmunoResearch, West Grove, PA, USA) followed by measurement of the HRP activity using a tetramethylbenzidine substrate solution (TMB, Sigma-Aldrich Chemie GmbH, Buchs, Switzerland).

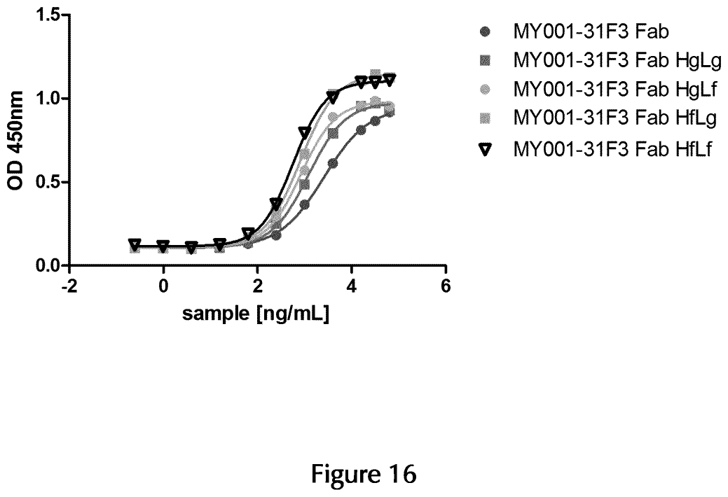

The results are shown in FIG. 16. All framework engineered versions of the antibody show similar or even improved binding to pS403/404 TDP-43. Accordingly, no impairment of antibody binding due the framework mutations could be identified.

### Example 13: Detection of TDP-43 Neoaggregates by Antibody 31F3 in Comparison to an Anti-pS403/404-TDP-43 Rabbit Polyclonal Antibody

Next, the antibody of the invention 31F3 was compared to a commercially available anti-pS403/404-TDP-43 rabbit polyclonal antibody (CosmoBio, Catalog No. TIP-PTDP-P05), which—according to its data sheet—was produced essentially as described in Hasegawa M, Arai T, Nonaka T, Kametani F, Yoshida M, Hashizume Y, Beach T G, Buratti E, Baralle F, Morita M, Nakano I, Oda T, Tsuchiya K, Akiyama H. Phosphorylated TDP-43 in frontotemporal lobar degeneration and amyotrophic lateral sclerosis. Ann Neurol. 2008 Jun. 10; 64(1):60-70.

To compare the ability of 31F3 and pS403/404 TDP-43 rabbit polyclonal (CosmoBio cat n.TIP-PTDP-P05) to detect pathological aggregates, the HEK293 seeding model, as described above in Example 7, was used.

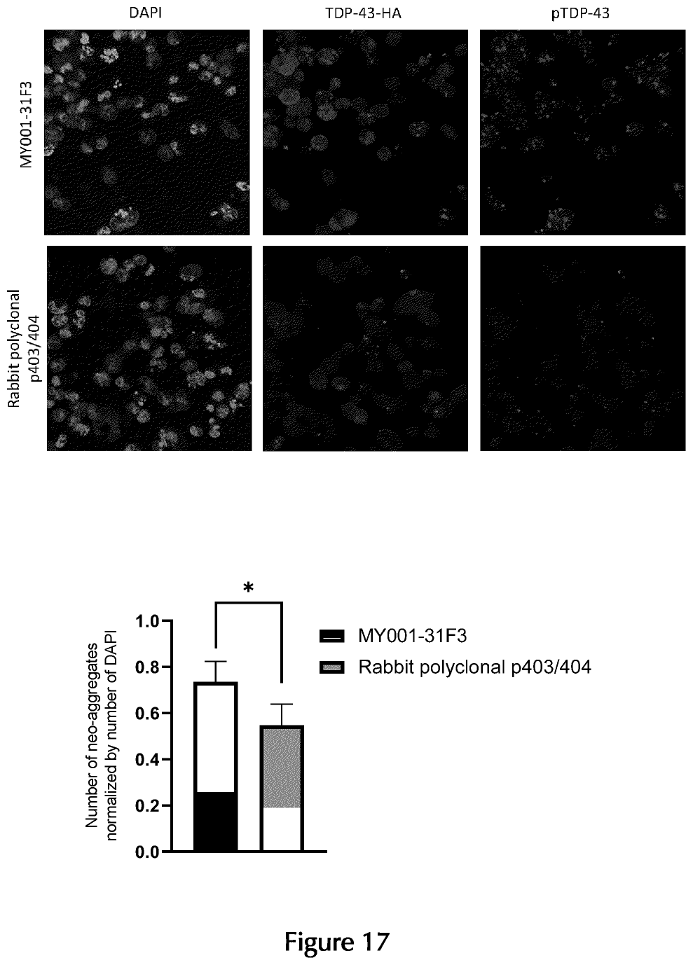

Results are shown in FIG. 17. Quantification of neoaggregates, defined as phosphorylated, cytoplasmic HA-tagged TDP-43 (TDP-43-HA), showed a larger number of neoaggregates detected by the antibody 31F3 when compared to the number detected by the pS403/404 TDP-43 rabbit polyclonal antibody, showing a higher sensitivity of 31F3 over the pS403/404 TDP-43 rabbit polyclonal antibody.

### Example 14: Binding and Specificity of Antibodies 31F3 and 30E1 in Comparison to an Anti-pS403/404-TDP-43 Rabbit Polyclonal Antibody and to an Anti-pS403/404-TDP-43 Mouse Monoclonal Antibody

Antibodies 31F3 (MY001-31F3) and 30E1 (MY001-30E1) were compared to a commercially available anti-pS403/404-TDP-43 rabbit polyclonal antibody (CosmoBio, Catalog No. TIP-PTDP-P05) and to a commercially available anti-pS403/404-TDP-43 mouse monoclonal antibody (Proteintech 66079-1-Ig). The pS403/404 TDP-43 rabbit polyclonal antibody was raised by immunizing a rabbit with the peptide NGGFGS(p)S(p)MDSK as in Hasegawa et al., 2008 (see also Example 13). To this end, binding of 31F3, 30E1, pS403/404 TDP-43 rabbit polyclonal (CosmoBio cat n.TIP-PTDP-P05) and pS403/404 TDP-43 mouse monoclonal (Proteintech 66079-1-Ig) were tested by ELISA against recombinant non-phosphorylated full-length TDP-43, phosphorylated pS403/404 TDP-43 (“peptide 2” as described above), and to pS409/410 TDP-43 (“peptide 1” as described above), and to a non-phosphorylated control peptide.

Briefly, 96 well microplates (Costar®, Corning incorporated, Corning, NY, USA) were coated with synthetic pTDP-43 peptides (0.5 μg/ml pS403/404 TDP-43 or 0.5 μg/ml pS409/410 TDP-43, Schafer-N or 0.5 μg/ml non-phosphorylated peptide, Schafer-N) or with non-phosphorylated full-length TDP-43 (SEQ ID NO: 1). Plates were washed with PBS-Tween 0.05% and blocked 1 h at room temperature with PBS containing 2% bovine serum albumin (BSA, Sigma-Aldrich Chemie GmbH, Buchs, Switzerland). Antibody preparations were incubated for 2 h at room temperature. Binding of human IgG or murine IgG or rabbit IgG to the antigen of interest was determined using a HRP-conjugated anti human antibody (anti-IgG-HRP from Jackson ImmunoResearch, West Grove, PA, USA) or HRP-conjugated anti-mouse antibody (goat anti-mouse IgG-HRP, Jackson ImmunoResearch, West Grove, PA, USA) or HRP-conjugated anti-rabbit antibody (goat anti-rabbit IgG-HRP, Jackson ImmunoResearch, West Grove, PA, USA) followed by measurement of the HRP activity using a tetramethylbenzidine substrate solution (TMB, Sigma-Aldrich Chemie GmbH, Buchs, Switzerland).

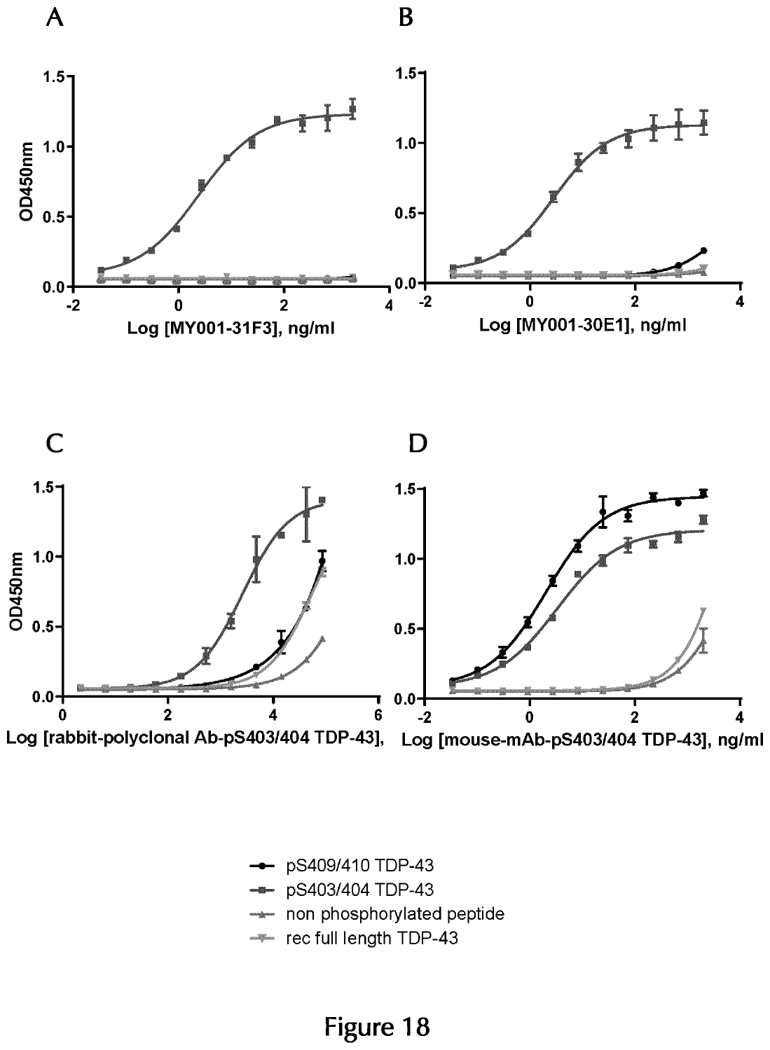

Results are shown in FIG. 18 and Table 6 below. The ELISA shows that the pS403/404 TDP-43 rabbit polyclonal antibody has a very low affinity as shown by the >2 LOG difference in EC50 when compared to the antibodies 30E1 and 31F3. The commercially available pS403/404 TDP-43 mouse monoclonal antibody shows no specificity for the p403/404 TDP-43 site, as it is also binding pS409/410 TDP-43 peptide.

TABLE 6
EC₅₀ values obtained pfor 31F3, 30E1 and comparative
antibodies to pS403/404-TDP-43.

| Antibody | EC₅₀ (ng/ml) |
|---|---|
| 31F3 | 2.44 |
| 30E1 | 2.809 |
| rabbit polyclonal antibody, CosmoBio | 2604 |
| mouse monoclonal antibody, Proteintech | 3.357 |

### Example 15: In Vivo Treatment of Mice with Antibodies 31F3 and 30E1 in a Mouse Model of TDP-43 Pathology

To investigate in vivo treatment effects of antibodies 31F3 (MY001-31F3) and 30E1 (MY001-30E1), a mouse model of TDP-43 pathology was used, namely, rNLS8 mice as described in Walker et al. 2015 (Walker A K, Spiller K J, Ge G, Zheng A, Xu Y, Zhou M, Tripathy K, Kwong L K, Trojanowski J Q, Lee V M. Functional recovery in new mouse models of ALS/FTLD after clearance of pathological cytoplasmic TDP-43. Acta Neuropathol. 2015 November; 130(5):643-60. doi: 10.1007/s00401-015-1460-x).

Brain Homogenates from Treated Mice:

rNLS8 mice were obtained as described (Walker et al. 2015). In brief NEFH-tTA (B6;C3-Tg(NEFH-tTA)8Vle/J) were crossed to hTDP-43-ΔNLS (B6; C3-Tg(tetO-TARDBP*)4Vle/J) mice and kept on chow diet with 200 mg/kg Doxycycline (Bio-Serv Cat #S3888). hTDP-43 expression was induced by switching 6-week old mice to a non-Dox diet. Wildtype (wt) or transgenic mice, 5.5 weeks after transgene induction, were injected intraperitoneally with 50 mg/kg of human IgG1 MY001-31F3, MY001-30E1, isotype control, or PBS (4 animals per group). 24 h after injection, mice were perfused with 30 mL DPBS and brain was dissected and an hemisphere was snap frozen for further biochemical analysis, and matching plasma samples were collected.

30 mg of mouse brain samples were transferred into the 2 ml-tube from the lysing kit, containing a mixture of ceramic beads with a diameter of 1.4 and 2.8 mm (Precellys, catalog number: P000918-LYSK0-A) and buffer (20 mM Tris-HCl pH 7.4, 300 mM NaCl, 1 mM EDTA, 1% NP-40) or B (20 mM Tris-HCl pH 7.4, 300 mM NaCl, 1 mM EDTA, 2 mM DTT) was added in a ratio of 5:1 to the amount of tissue, and completed with protease inhibitor (complete mini-EDTA free tablets) and phosphatase inhibitor (PhosSTOP). The samples were homogenized in a Minilys device three times for 30 s each at maximum speed. Cool down the sample on ice between rounds.

Quantification of Antibody in the Brain:

High-binding half-area 96-well microplates (Greiner #675061) were coated with 1 ug/mL anti-human IgG (donkey anti-hu IgG Jackson ImmunoResearch #709-005-149) for 1 h RT. Plates were washed once in PBS 0.05% Tween 20 (Sigma #1379-500 ml) and blocked in PBS 2% BSA (Sigma, A8022) for 1 h RT. Brain homogenate were diluted 1:50 in PBS 0.5% BSA. Samples were incubated together with standard human IgG1 for 1 h30 min at RT. Plates were washed 4 times in PBS 0.05% Tween 20. Anti-human fc HRP (Jackson ImmunoResearch #109-035-098) was diluted 1:4000 and plates were washed 4 times in PBS 0.05% Tween 20. TMB solution (Sigma, T2885) was added and incubated for 5 min. Reaction was stopped by adding IM H2SO4 (AppliChem, A2699) and the plate were immediately read on a Multiskan FC microplate photometer (Thermo Fisher, REF 51119000) at 450 nm. Standard curve was fitted in Graphpad prism using sigmoidal, 4PL (four parameter logistic), and sample concentration was calculated.

Measurement of Target Engagement and Effect on TDP-43 Distribution:

Brain homogenized in buffer B was subjected to the Sarkospin method to separate soluble proteins and insoluble aggregates, and further analyzed by Western blotting as described in Example 4 and Example 7. Target engagement was measured by looking at the presence of human antibody heavy chain in the TDP-43 aggregate fraction. Modulation of TDP-43 aggregates was studies by measuring the amount of total TDP-43 in the soluble fraction or the amount of pathological TDP-43 pS409/410 in the insoluble fraction. Primary antibodies were: anti-human IgG (Jackson Immuno Research, 109-035-098), TDP-43 (3H8, Novus Bio), pTDP43 409/410 (CosmoBio, CAC-TIP-PTD-M01), SOD1 (Enzo Life Sciences, ADI-SOD-100 J/F). Signal was quantified by band densitometry using Fiji software, and data was normalized to SOD 1.

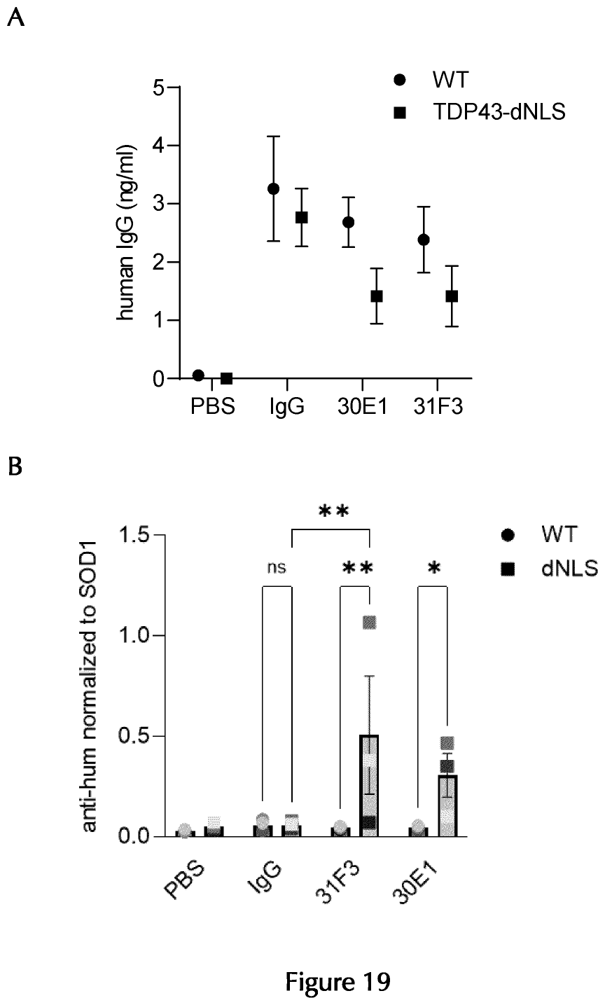

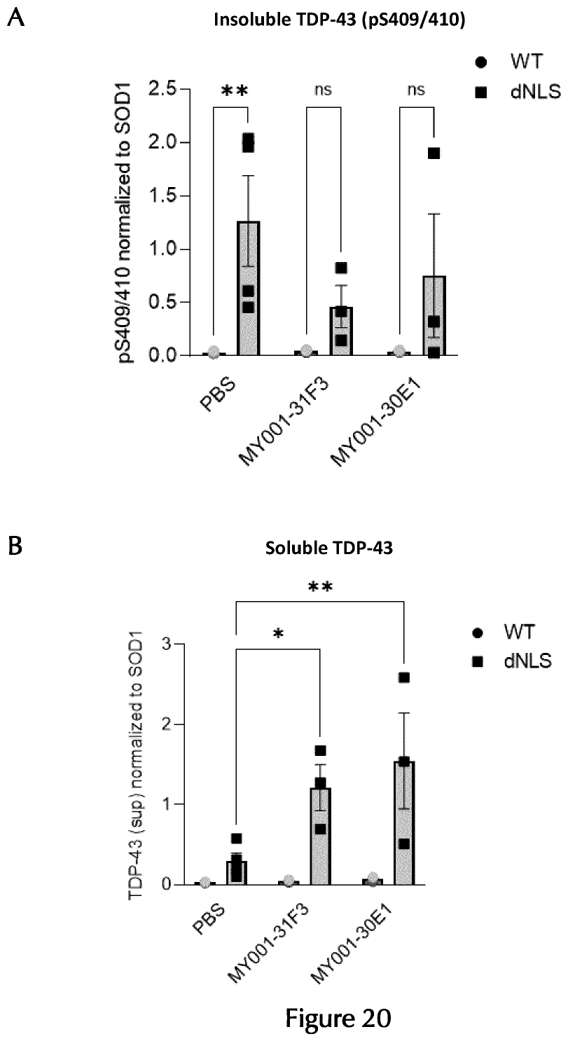

Results are shown in FIGS. 19 and 20. The data shown in FIG. 19 demonstrate that the antibodies 31F3 and 30E1 (injected intraperitoneally) can reach the brain (FIG. 19 A) and bind to TDP-43 aggregates (FIG. 19 B). The data shown in FIG. 20 demonstrate a significant increase over vehicle in soluble TDP-43 (FIG. 20 B) and a decrease in phosphorylated TDP-43 (FIG. 20 A), indicating that the antibodies 31F3 and 30E1 are able to counteract pathological aggregation of TDP-43.

TABLE OF SEQUENCES AND SEQ ID NUMBERS (SEQUENCE LISTING):

| SEQ ID NO | Sequence | Remarks |
|---|---|---|
| SEQ ID NO: 1 | MSEYIRVTEDENDEPIEIPSEDDGTVLLSTVTAQFP | human TDP-43 |
| | GACGLRYRNPVSQCMRGVRLVEGILHAPDAG | |
| | WGNLVYVVNYPKDNKRKMDETDASSAVKVKR | |
| | AVQKTSDLIVLGLPWKTTEQDLKEYFSTFGEVLM | |
| | VQVKKDLKTGHSKGFGFVRFTEYETQVKVMSQ | |
| | RHMIDGRWCDCKLPNSKQSQDEPLRSRKVFVG | |
| | RCTEDMTEDELREFFSQYGDVMDVFIPKPFRAFA | |
| | FVTFADDQIAQSLCGEDLIIKGISVHISNAEPKHN | |
| | SNRQLERSGRFGGNPGGFGNQGGFGNSRGGG | |
| | AGLGNNQGSNMGGGMNFGAFSINPAMMAAA | |
| | QAALQSSWGMMGMLASQQNQSGPSGNNQN | |
| | QGNMQREPNQAFGSGNNSYSGSNSGAAIGW | |
| | GSASNAGSGSGFNGGFGSSMDSKSSGWGM | |
| SEQ ID NO: 2 | NAGSGSGFNGGFGSSMDSKSSGWGM | TDP-43 peptide |

31F3

| | | |
|---|---|---|
| SEQ ID NO: 3 | GYYLH | CDRH1 |
| SEQ ID NO: 4 | RINPGSGGTKYAQKFQG | CDRH2 |
| SEQ ID NO: 5 | YQVYGERGRYMDV | CDRH3 |
| SEQ ID NO: 6 | TGTSSDVGTYDLVS | CDRL1 |
| SEQ ID NO: 7 | EDNKRPS | CDRL2 |
| SEQ ID NO: 8 | CSYAAISTLI | CDRL3 |
| SEQ ID NO: 9 | QVQLEQSGAEVKRPGASVKVSCKASGYTFTGYY | VH |
| | LHWVRQAPGQGLEWMGRINPGSGGTKYAQKF | |
| | QGRVSMTRDTSISTAYMEVNRLRPDDTAVYYCA | |
| | RYQVYGERGRYMDVWGKGTTVTVSS | |
| SEQ ID NO: 10 | QSALTQPASVSGSPGQSITISCTGTSSDVGTYDL | VL |
| | VSWYQQHPGKAPKLIIYEDNKRPSGLSNRFSGSK | |
| | SGNTASLTISGLQADDEADYYCCSYAAISTLIFGG | |
| | GTKVTVL | |
| SEQ ID NO: 11 | QVQLEQSGAEVKRPGASVKVSCKASGYTFTGYY | heavy chain |
| | LHWVRQAPGQGLEWMGRINPGSGGTKYAQKF | |
| | QGRVSMTRDTSISTAYMEVNRLRPDDTAVYYCA | |
| | RYQVYGERGRYMDVWGKGTTVTVSSASTKGPS | |
| | VFPLAPCSRSTSGGTAALGCLVKDYFPEPVTVSW | |
| | NSGALTSGVHTFPAVLQSSGLYSLSSVVTVPSSSL | |
| | GTQTYTCNVNHKPSNTKVDKRVELKTPLGDTT | |
| | HTCPRCPEPKSCDTPPPCPRCPEPKSCDTPPPCPR | |
| | CPEPKSCDTPPPCPRCPAPELLGGPSVFLFPPKPK | |
| | DTLMISRTPEVTCVVVDVSHEDPEVQFKWYVDG | |
| | VEVHNAKTKPREEQFNSTFRVVSVLTVLHQDWL | |
| | NGKEYKCKVSNKALPAPIEKTISKTKGQPREPQVY | |
| | TLPPSREEMTKNQVSLTCLVKGFYPSDIAVEWESS | |
| | GQPENNYNTTPPMLDSDGSFFLYSKLTVDKSRW | |
| | QQGNIFSCSVMHEALHNRFTQKSLSLSPGK* | |
| SEQ ID NO: 12 | QSALTQPASVSGSPGQSITISCTGTSSDVGTYDL | light chain |
| | VSWYQQHPGKAPKLIIYEDNKRPSGLSNRFSGSK | |
| | SGNTASLTISGLQADDEADYYCCSYAAISTLIFGG | |
| | GTKVTVLGQPKAAPSVTLFPPSSEELQANKATLV | |
| | CLISDFYPGAVTVAWKADSSPVKAGVETTTPSKQ | |
| | SNNKYAASSYLSLTPEQWKSHRSYSCQVTHEGST | |
| | VEKTVAPTECS* | |

30E1 (30E3)

| | | |
|---|---|---|
| SEQ ID NO: 13 | DYYLH | CDRH1 |
| SEQ ID NO: 14 | RINPNSGNTNYAQKFQG | CDRH2 |
| SEQ ID NO: 15 | VKIVVLRSTPTLYYFDY | CDRH3 |
| SEQ ID NO: 16 | EGSKRPS | CDRL2 |
| SEQ ID NO: 17 | CSYAGTSTLV | CDRL3 |
| SEQ ID NO: 18 | QVQLVQSGAEVKKPGASVKVSCKTSGYTFTDYY | VH |
| | LHWVRQAPGQGLEWMGRINPNSGNTNYAQK | |
| | FQGRVSVTRDTSISTAYMELSRLSSDDTAVYFCA | |
| | RVKIVVLRSTPTLYYFDYWGQGTLVTVSS | |
| SEQ ID NO: 19 | QSALTQPASVSGSPGQSITISCTGTSSDVGTYDL | VL |
| | VSWYQQHPGKAPKVMIYEGSKRPSGVSNRFSGS | |
| | KSGNTASLTISGLQAEDEADYYCCSYAGTSTLVF | |
| | GGGTKVTVL | |
| SEQ ID NO: 20 | QVQLVQSGAEVKKPGASVKVSCKTSGYTFTDYY | heavy chain |
| | LHWVRQAPGQGLEWMGRINPNSGNTNYAQK | |
| | FQGRVSVTRDTSISTAYMELSRLSSDDTAVYFCA | |
| | RVKIVVLRSTPTLYYFDYWGQGTLVTVSSASTKG | |
| | PSVFPLAPCSRSTSGGTAALGCLVKDYFPEPVTVS | |
| | WNSGALTSGVHTFPAVLQSSGLYSLSSVVTVPSS | |
| | SLGTQTYTCNVNHKPSNTKVDKRVELKTPLGDT | |
| | THTCPRCPEPKSCDTPPPCPRCPEPKSCDTPPPCP | |
| | RCPAPELLGGPSVFLFPPKPKDTLMISRTPEVTCV | |
| | VVDVSHEDPEVQFKWYVDGVEVHNAKTKPREE | |
| | QFNSTFRVVSVLTVLHQDWLNGKEYKCKVSNK | |
| | ALPAPIEKTISKTKGQPREPQVYTLPPSREEMTKN | |
| | QVSLTCLVKGFYPSDIAVEWESSGQPENNYNTTP | |
| | PMLDSDGSFFLYSKLTVDKSRWQQGNIFSCSVM | |
| | HEALHNRFTQKSLSLSPGK* | |
| SEQ ID NO: 21 | QSALTQPASVSGSPGQSITISCTGTSSDVGTYDL | light chain |
| | VSWYQQHPGKAPKVMIYEGSKRPSGVSNRFSGS | |
| | KSGNTASLTISGLQAEDEADYYCCSYAGTSTLVF | |
| | GGGTKVTVLGQPKAAPSVTLFPPSSEELQANKAT | |
| | LVCLISDFYPGAVTVAWKADSSPVKAGVETTTPS | |
| | KQSNNKYAASSYLSLTPEQWKSHRSYSCQVTHE | |
| | GSTVEKTVAPTECS* | |

9F11

| | | |
|---|---|---|
| SEQ ID NO: 22 | RINPKSGNTNYAQKFQG | CDRH2 |
| SEQ ID NO: 23 | VKIVVVRSTPTLYYLDY | CDRH3 |
| SEQ ID NO: 24 | TGSSSDVGTYDLVS | CDRL1 |
| SEQ ID NO: 25 | EGHKRPS | CDRL2 |
| SEQ ID NO: 26 | QVQLVQSGAEVKKPGASVKVSCKTSGYTFTDYY | VH |
| | LHWVRQAPGQGLEWMGRINPKSGNTNYAQKF | |
| | QGRVTVTRDTSISTAYMELSRLRSDDTAVYYCAR | |
| | VKIVVVRSTPTLYYLDYWGQGTLVTVSS | |
| SEQ ID NO: 27 | QSALTQPASVSGSPGQSITISCTGSSSDVGTYDL | VL |
| | VSWYQQHPGKAPKVMIYEGHKRPSGVSNRVSG | |
| | SKSGNTASLTISGLQAEDEADYYCCSYAGTSTLVF | |
| | GGGTKVTVL | |

15D7

| | | |
|---|---|---|
| SEQ ID NO: 28 | NYAMH | CDRH1 |
| SEQ ID NO: 29 | AMSSNGGSTYYGDSVKG | CDRH2 |
| SEQ ID NO: 30 | RGRKAPSLYYFDC | CDRH3 |
| SEQ ID NO: 31 | RASQSVSSNLA | CDRL1 |
| SEQ ID NO: 32 | GASTRAT | CDRL2 |
| SEQ ID NO: 33 | QQYNNWPPWT | CDRL3 |
| SEQ ID NO: 34 | EVQLVESGGGLVQPGGSLRLSCSASGFTFGNYA | VH |
| | MHWVRQAPGKGLEYVSAMSSNGGSTYYGDSV | |
| | KGRFTISRDNSKNTLYLQMSSLRAEDTAVYYCVT | |
| | RGRKAPSLYYFDCWGPGTLVTVSS | |
| SEQ ID NO: 35 | EIVLTQSPATLSVSPGERATLSCRASQSVSSNLAW | VL |
| | YQQKPGQAPRLLIYGASTRATGIPARFSGSGSGT | |
| | EFTLTISSLQSEDFAVYYCQQYNNWPPWTFGQG | |
| | TRLEIK | |

Variant CDRs

| | | |
|---|---|---|
| SEQ ID NO: 36 | RINPNAGNTNYAQKFQG | CDRH2 |
| SEQ ID NO: 37 | RINPKAGNTNYAQKFQG | CDRH2 |
| SEQ ID NO: 38 | RINPNSGNVNYAQKFQG | CDRH2 |
| SEQ ID NO: 39 | RINPNSGNANYAQKFQG | CDRH2 |
| SEQ ID NO: 40 | RINPKSGNVNYAQKFQG | CDRH2 |
| SEQ ID NO: 41 | RINPKSGNANYAQKFQG | CDRH2 |
| SEQ ID NO: 42 | RINPGSGGTRYAQKFQG | CDRH2 |
| SEQ ID NO: 43 | RINPGSGGTQYAQKFQG | CDRH2 |
| SEQ ID NO: 44 | RINPGSGGTNYAQKFQG | CDRH2 |
| SEQ ID NO: 45 | RINPGSGGTKYAQRFQG | CDRH2 |
| SEQ ID NO: 46 | RINPGSGGTKYAQQFQG | CDRH2 |
| SEQ ID NO: 47 | RINPNSGNTNYAQRFQG | CDRH2 |
| SEQ ID NO: 48 | RINPNSGNTNYAQQFQG | CDRH2 |
| SEQ ID NO: 49 | RINPKSGNTNYAQRFQG | CDRH2 |
| SEQ ID NO: 50 | RINPKSGNTNYAQQFQG | CDRH2 |
| SEQ ID NO: 51 | VRIVVLRSTPTLYYFDY | CDRH3 |
| SEQ ID NO: 52 | VRIVVVRSTPTLYYLDY | CDRH3 |
| SEQ ID NO: 53 | ELNKRPS | CDRL2 |
| SEQ ID NO: 54 | EQNKRPS | CDRL2 |
| SEQ ID NO: 55 | EANKRPS | CDRL2 |
| SEQ ID NO: 56 | EDNRRPS | CDRL2 |
| SEQ ID NO: 57 | EDNQRPS | CDRL2 |
| SEQ ID NO: 58 | EGSRRPS | CDRL2 |
| SEQ ID NO: 59 | EGSQRPS | CDRL2 |
| SEQ ID NO: 60 | EGHRRPS | CDRL2 |
| SEQ ID NO: 61 | EGHQRPS | CDRL2 |
| SEQ ID NO: 62 | SSYAAISTLI | CDRL3 |
| SEQ ID NO: 63 | SSYAGTSTLV | CDRL3 |
| SEQ ID NO: 64 | QSYAAISTLI | CDRL3 |
| SEQ ID NO: 65 | QSYAGTSTLV | CDRL3 |
| SEQ ID NO: 66 | GSYAAISTLI | CDRL3 |
| SEQ ID NO: 67 | GSYAGTSTLV | CDRL3 |

Constant regions

| | | |
|---|---|---|
| SEQ ID NO: 68 | ASTKGPSVFPLAPSSKSTSGGTAALGCLVKDYFPE | heavy chain |
| | PVTVSWNSGALTSGVHTFPAVLQSSGLYSLSSVV | (CH1-CH2-CH3) |
| | TVPSSSLGTQTYICNVNHKPSNTKVDKKVEPKSC | |
| | DKTHTCPPCPAPELLGGPSVFLFPPKPKDTLMISR | |
| | TPEVTCVVVDVSHEDPEVKFNWYVDGVEVHNA | |
| | KTKPREEQYNSTYRVVSVLTVLHQDWLNGKEYK | |
| | CKVSNKALPAPIEKTISKAKGQPREPQVYTLPPSR | |
| | DELTKNQVSLTCLVKGFYPSDIAVEWESNGQPE | |
| | INNYKTTPPVLDSDGSFFLYSKLTVDKSRWQQG | |
| | NVFSCSVMHEALHNHYTQKSLSLSPGK* | |
| SEQ ID NO: 69 | GQPKAAPSVTLFPPSSEELQANKATLVCLISDFYP | lambda light chain |
| | GAVTVAWKADSSPVKAGVETTTPSKQSNNKYA | (CL) |
| | ASSYLSLTPEQWKSHRSYSCQVTHEGSTVEKTVA | |
| | PTECS* | |
| SEQ ID NO: 70 | RTVAAPSVFIFPPSDEQLKSGTASVVCLLNNFYPR | kappa light chain |
| | EAKVQWKVDNALQSGNSQESVTEQDSKDSTYS | (CL) |
| | LSSTLTLSKADYEKHKVYACEVTHQGLSSPVTKSF | |
| | NRGEC* | |

Linkers, localization sequences and tags

| | | |
|---|---|---|
| SEQ ID NO: 71 | GGGGS | peptide linker motif |
| SEQ ID NO: 72 | KDEL | retention peptide |
| SEQ ID NO: 73 | PKKKRKV | nuclear localization |
| | | sequence |
| SEQ ID NO: 74 | DYKDHDGDYKDHDI-DYKDDDDK | s3Flag |
| SEQ ID NO: 75 | YPYDVPDYA | HA tag |

Nucleic acid sequences
31F3

| | | |
|---|---|---|
| SEQ ID NO: 76 | GGCTACTATTTACAC | CDRH1 |
| SEQ ID NO: 77 | CGGATCAACCCTGGCAGTGGTGGCACAAAG | CDRH2 |
| | TATGCACAGAAGTTTCAGGGC | |
| SEQ ID NO: 78 | TACCAAGTGTACGGTGAGCGCGGGAGGTAC | CDRH3 |
| | ATGGACGTC | |
| SEQ ID NO: 79 | ACTGGAACCAGCAGTGATGTTGGGACTTATG | CDRL1 |
| | ACCTTGTCTCC | |
| SEQ ID NO: 80 | GAAGACAATAAGCGGCCCTCA | CDRL2 |
| SEQ ID NO: 81 | TGCTCATATGCTGCTATTAGCACTTTAATA | CDRL3 |
| SEQ ID NO: 82 | CAGGTGCAGCTGGTGCAGTCTGGGGCTGAG | VH |
| | GTGAAGAAACCTGGGGCCTCAGTGAAGGTC | |
| | TCCTGCAAGACTTCTGGATACACCTTCACCGA | |
| | CTACTATTTACACTGGGTGCGACAGGCCCCT | |
| | GGACAAGGGCTTGAGTGGATGGGACGGATC | |
| | AACCCTAACAGTGGTAACACAAACTATGCAC | |
| | AGAAGTTTCAGGGCCGGGTCAGCGTGACCA | |
| | GGGACACGTCCATCAGCACAGCCTACATGGA | |
| | GCTGAGCAGGCTGAGCTCTGACGACACGGC | |
| | CGTGTATTTCTGTGCGAGAGTTAAGATAGTA | |
| | GTTCTAAGGAGCACGCCAACGCTCTACTACTT | |
| | TGACTACTGGGGCCAGGGAACCCTGGTCAC | |
| | CGTGTCCTCA | |
| SEQ ID NO: 83 | CAGTCTGCCCTGACTCAGCCTGCCTCCGTGT | VL |
| | CTGGGTCTCCTGGACAGTCGATCACCATCTC | |
| | CTGCACTGGAACCAGCAGTGATGTTGGGACT | |
| | TATGACCTTGTCTCCTGGTACCAACAGCACCC | |
| | AGGCAAAGCCCCCAAACTCATCATTTATGAA | |
| | GACAATAAGCGGCCCTCAGGGCTTTCTAATC | |
| | GCTTCTCTGGCTCCAAGTCTGGCAACACGGC | |
| | CTCCCTGACGATCTCTGGGCTCCAGGCTGAC | |
| | GACGAGGCTGATTATTACTGCTGCTCATATGC | |
| | TGCTATTAGCACTTTAATATTCGGCGGAGGG | |
| | ACCAAGGTGACCGTCCTA | |
| SEQ ID NO: 84 | TATCCTGGNNGAGGGCATGGGGAATCTTCTT | heavy chain |
| | NTNGGGGGCGGCAGNCACACGAGCGCACT | |
| | CCCAGGTGCAGCTGGAGCAGTCTGGGGCTG | |
| | AGGTGAAGAGGCCTGGGGCCTCAGTGAAGG | |
| | TCTCCTGCAAGGCTTCTGGATACACCTTCACC | |
| | GGCTACTATTTACACTGGGTGCGACAGGCCC | |
| | CTGGACAAGGACTTGAGTGGATGGGACGGA | |
| | TCAACCCTGGCAGTGGTGGCACAAAGTATGC | |
| | ACAGAAGTTTCAGGGCAGGGTCAGCATGAC | |
| | CCGGGACACGTCCATCAGCACAGCCTATATG | |
| | GAGGTGAACAGACTGAGACCTGACGACACG | |
| | GCCGTGTATTATTGTGCGAGATACCAAGTGT | |
| | ACGGTGAGCGCGGGAGGTACATGGACGTCT | |
| | GGGGCAAAGGGACCACGGTCACCGTCTCGT | |
| | CAGCTTCCACCAAGGGCCCATCGGTCTTCCC | |
| | CCTGGCGCCCTGCTCCAGGAGCACCTCTGG | |
| | GGGCACAGCGGCCCTGGGCTGCCTGGTCAA | |
| | GGACTACTTCCCCGAACCGGTGACGGTGTCG | |
| | TGGAACTCAGGCGCCCTGACCAGCGGCGTG | |
| | CACACCTTCCCGGCTGTCCTACAGTCCTCAG | |
| | GACTCTACTCCCTCAGCAGCGTGGTGACCGT | |
| | GCCCTCCAGCAGCTTGGGCACCCAGACCTAC | |
| | ACCTGCAACGTGAATCACAAGCCCAGCAACA | |
| | CCAAGGTGGACAAGAGAGTTGAGCTCAAAA | |
| | CCCCACTTGGTGACACAACTCACACATGCCC | |
| | ACGGTGCCCAGAGCCCAAATCTTGTGACACA | |
| | CCTCCCCCGTGCCCACGGTGCCCAGAGCCCA | |
| | AATCTTGTGACACACCTCCCCCATGCCCACG | |
| | GTGCCCAGAGCCCAAATCTTGTGACACACCT | |
| | CCCCCATGCCCACGGTGCCCAGCACCTGAAC | |
| | TCCTGGGAGGACCGTCAGTCTTCCTCTTCCCC | |
| | CCAAAACCCAAGGATACCCTTATGATTTCCCG | |
| | GACCCCTGAGGTCACGTGCGTGGTGGTGGA | |
| | CGTGAGCCACGAAGACCCCGAGGTCCAGTT | |
| | CAAGTGGTACGTGGACGGCGTGGAGGTGCA | |
| | TAATGCCAAGACAAAGCCGCGGGAGGAGCA | |
| | GTTCAACAGCACGTTCCGTGTGGTCAGCGTC | |
| | CTCACCGTCCTGCACCAGGACTGGCTGAACG | |
| | GCAAGGAGTACAAGTGCAAGGTCTCCAACAA | |
| | AGCCCTCCCAGCCCCCATCGAGAAAACCATC | |
| | TCCAAAACCAAAGGACAGCCCCGAGAACCAC | |
| | AGGTGTACACCCTGCCCCCATCCCGGGAGG | |
| | AGATGACCAAGAACCAGGTCAGCCTGACCTG | |
| | CCTGGTCAAAGGCTTCTACCCCAGCGACATC | |
| | GCCGTGGAGTGGGAGAGCAGCGGGCAGCC | |
| | GGAGAACAACTACAACACCACGCCTCCCATG | |
| | CTGGACTCCGACGGCTCCTTCTTCCTCTACAG | |
| | CAAGCTCACCGTGGACAAGAGCAGGTGGCA | |
| | GCAGGGGAACATCTTCTCATGCTCCGTGATG | |
| | CATGAGGCTCTGCACAACCGCTTCACGCAGA | |
| | AGAGCCTCTCCCTGTCTCCGGGTAAATGAGT | |
| | GCGACGGCCGGCAAGCCCCCGCTCCCCGGG | |
| | CTCTCGGGGTCGCGCGAGGATGCTTGGCAC | |
| SEQ ID NO: 85 | ACCTCNGGCACAGGGTCCTGGGCCCAGTCT | light chain |
| | GCCCTGACTCAGCCTGCCTCCGTGTCTGGGT | |
| | CTCCTGGACAGTCGATCACCATCTCCTGCACT | |
| | GGAACCAGCAGTGATGTTGGGACTTATGACC | |
| | TTGTCTCCTGGTACCAACAGCACCCAGGCAA | |
| | AGCCCCCAAACTCATCATTTATGAAGACAATA | |
| | AGCGGCCCTCAGGGCTTTCTAATCGCTTCTCT | |
| | GGCTCCAAGTCTGGCAACACGGCCTCCCTGA | |
| | CGATCTCTGGGCTCCAGGCTGACGACGAGG | |
| | CTGATTATTACTGCTGCTCATATGCTGCTATTA | |
| | GCACTTTAATATTCGGCGGAGGGACCAAGGT | |
| | GACCGTCCTAGGTCAGCCCAAGGCTGCCCCC | |
| | TCGGTCACTCTGTTCCCGCCCTCCTCTGAGGA | |
| | GCTTCAAGCCAACAAGGCCACACTGGTGTGT | |
| | CTCATAAGTGACTTCTACCCGGGAGCCGTGA | |
| | CAGTGGCCTGGAAGGCAGATAGCAGCCCCG | |
| | TCAAGGCGGGAGTGGAGACCACCACACCCT | |
| | CCAAACAAAGCAACAACAAGTACGCGGCCA | |
| | GCAGCTATCTGAGCCTGACGCCTGAGCAGTG | |
| | GAAGTCCCACAGAAGCTACAGCTGCCAGGTC | |
| | ACGCATGAAGGGAGCACCGTGGAGAAGACA | |
| | GTGGCCCCTACAGAATGTTCATAGGTTCTAAA | |
| | CCCTCACCCCCCCCACGGGAGACTAGAGCTG | |
| | A | |

30E1 (30E3)

| | | |
|---|---|---|
| SEQ ID NO: 86 | GACTACTATTTACAC | CDRH1 |
| SEQ ID NO: 87 | CGGATCAACCCTAACAGTGGTAACACAAACT | CDRH2 |
| | ATGCACAGAAGTTTCAGGGC | |
| SEQ ID NO: 88 | GTTAAGATAGTAGTTCTAAGGAGCACGCCAA | CDRH3 |
| | CGCTCTACTACTTTGACTAC | |
| SEQ ID NO: 89 | ACTGGAACCAGCAGTGATGTTGGGACTTATG | CDRL1 |
| | ACCTTGTCTCC | |
| SEQ ID NO: 90 | GAGGGCAGTAAGCGGCCCTCA | CDRL2 |
| SEQ ID NO: 91 | TGCTCATATGCAGGTACTAGCACCTTGGTG | CDRL3 |
| SEQ ID NO: 92 | CAGGTGCAGCTGGTGCAGTCTGGGGCTGAG | VH |
| | GTGAAGAAACCTGGGGCCTCAGTGAAGGTC | |
| | TCCTGCAAGACTTCTGGATACACCTTCACCGA | |
| | CTACTATTTACACTGGGTGCGACAGGCCCCT | |
| | GGACAAGGGCTTGAGTGGATGGGACGGATC | |
| | AACCCTAACAGTGGTAACACAAACTATGCAC | |
| | AGAAGTTTCAGGGCCGGGTCAGCGTGACCA | |
| | GGGACACGTCCATCAGCACAGCCTACATGGA | |
| | GCTGAGCAGGCTGAGCTCTGACGACACGGC | |
| | CGTGTATTTCTGTGCGAGAGTTAAGATAGTA | |
| | GTTCTAAGGAGCACGCCAACGCTCTACTACTT | |
| | TGACTACTGGGGCCAGGGAACCCTGGTCAC | |
| | CGTGTCCTCA | |
| SEQ ID NO: 93 | CAGTCTGCCCTGACTCAGCCTGCCTCCGTGT | VL |
| | CTGGGTCTCCTGGACAGTCGATCACCATCTC | |
| | CTGCACTGGAACCAGCAGTGATGTTGGGACT | |
| | TATGACCTTGTCTCCTGGTACCAACAACATCC | |
| | AGGCAAAGCCCCCAAAGTCATGATTTATGAG | |
| | GGCAGTAAGCGGCCCTCAGGGGTTTCTAATC | |
| | GCTTCTCTGGCTCCAAGTCTGGCAACACGGC | |
| | CTCCCTGACAATCTCCGGACTCCAGGCTGAG | |
| | GACGAGGCTGATTATTACTGCTGCTCATATGC | |
| | AGGTACTAGCACCTTGGTGTTCGGCGGAGG | |
| | GACCAAGGTGACCGTCCTA | |
| SEQ ID NO: 94 | ATCCTCTTCTTGGTGGCAGCAGCCACAGGAG | heavy chain |
| | CCCACTCCCAGGTGCAGCTGGTGCAGTCTGG | |
| | GGCTGAGGTGAAGAAACCTGGGGCCTCAGT | |
| | GAAGGTCTCCTGCAAGACTTCTGGATACACC | |
| | TTCACCGACTACTATTTACACTGGGTGCGACA | |
| | GGCCCCTGGACAAGGGCTTGAGTGGATGGG | |
| | ACGGATCAACCCTAACAGTGGTAACACAAAC | |
| | TATGCACAGAAGTTTCAGGGCCGGGTCAGC | |
| | GTGACCAGGGACACGTCCATCAGCACAGCCT | |
| | ACATGGAGCTGAGCAGGCTGAGCTCTGACG | |
| | ACACGGCCGTGTATTTCTGTGCGAGAGTTAA | |
| | GATAGTAGTTCTAAGGAGCACGCCAACGCTC | |
| | TACTACTTTGACTACTGGGGCCAGGGAACCC | |
| | TGGTCACCGTGTCCTCAGCTTCCACCAAGGG | |
| | CCCATCGGTCTTCCCCCTGGCGCCCTGCTCC | |
| | AGGAGCACCTCTGGGGGCACAGCGGCCCTG | |
| | GGCTGCCTGGTCAAGGACTACTTCCCCGAAC | |
| | CGGTGACGGTGTCGTGGAACTCAGGCGCCC | |
| | TGACCAGCGGCGTGCACACCTTCCCGGCTGT | |
| | CCTACAGTCCTCAGGACTCTACTCCCTCAGCA | |
| | GCGTGGTGACCGTGCCCTCCAGCAGCTTGG | |
| | GCACCCAGACCTACACCTGCAACGTGAATCA | |
| | CAAGCCCAGCAACACCAAGGTGGACAAGAG | |
| | AGTTGAGCTCAAAACCCCACTTGGTGACACA | |
| | ACTCACACATGCCCACGGTGCCCAGAGCCCA | |
| | AATCTTGTGACACACCTCCCCCGTGCCCACG | |
| | GTGCCCAGAGCCCAAATCTTGTGACACACCT | |
| | CCCCCATGCCCACGGTGCCCAGCACCTGAAC | |
| | TCCTGGGAGGACCGTCAGTCTTCCTCTTCCCC | |
| | CCAAAACCCAAGGATACCCTTATGATTTCCCG | |
| | GACCCCTGAGGTCACGTGCGTGGTGGTGGA | |
| | CGTGAGCCACGAAGACCCCGAGGTCCAGTT | |
| | CAAGTGGTACGTGGACGGCGTGGAGGTGCA | |
| | TAATGCCAAGACAAAGCCGCGGGAGGAGCA | |
| | GTTCAACAGCACGTTCCGTGTGGTCAGCGTC | |
| | CTCACCGTCCTGCACCAGGACTGGCTGAACG | |
| | GCAAGGAGTACAAGTGCAAGGTCTCCAACAA | |
| | AGCCCTCCCAGCCCCCATCGAGAAAACCATC | |
| | TCCAAAACCAAAGGACAGCCCCGAGAACCAC | |
| | AGGTGTACACCCTGCCCCCATCCCGGGAGG | |
| | AGATGACCAAGAACCAGGTCAGCCTGACCTG | |
| | CCTGGTCAAAGGCTTCTACCCCAGCGACATC | |
| | GCCGTGGAGTGGGAGAGCAGCGGGCAGCC | |
| | GGAGAACAACTACAACACCACGCCTCCCATG | |
| | CTGGACTCCGACGGCTCCTTCTTCCTCTACAG | |
| | CAAGCTCACCGTGGACAAGAGCAGGTGGCA | |
| | GCAGGGGAACATCTTCTCATGCTCCGTGATG | |
| | CATGAGGCTCTGCACAACCGCTTCACGCAGA | |
| | AGAGCCTCTCCCTGTCTCCGGGTAAATGAGT | |
| | GCGACGGCCGGCAAGCCCCCGCTCCCCGGG | |
| | CTCTCGGGGTCGCGCGAGGATGCTTGGCAC | |
| | GT | |
| SEQ ID NO: 95 | CACTCAGGGCACAGGGTCCTGGGCCCAGTC | light chain |
| | TGCCCTGACTCAGCCTGCCTCCGTGTCTGGG | |
| | TCTCCTGGACAGTCGATCACCATCTCCTGCAC | |
| | TGGAACCAGCAGTGATGTTGGGACTTATGAC | |
| | CTTGTCTCCTGGTACCAACAACATCCAGGCAA | |
| | AGCCCCCAAAGTCATGATTTATGAGGGCAGT | |
| | AAGCGGCCCTCAGGGGTTTCTAATCGCTTCT | |
| | CTGGCTCCAAGTCTGGCAACACGGCCTCCCT | |
| | GACAATCTCCGGACTCCAGGCTGAGGACGA | |
| | GGCTGATTATTACTGCTGCTCATATGCAGGTA | |
| | CTAGCACCTTGGTGTTCGGCGGAGGGACCA | |
| | AGGTGACCGTCCTAGGTCAGCCCAAGGCTG | |
| | CCCCCTCGGTCACTCTGTTCCCGCCCTCCTCT | |
| | GAGGAGCTTCAAGCCAACAAGGCCACACTG | |
| | GTGTGTCTCATAAGTGACTTCTACCCGGGAG | |
| | CCGTGACAGTGGCCTGGAAGGCAGATAGCA | |
| | GCCCCGTCAAGGCGGGAGTGGAGACCACCA | |
| | CACCCTCCAAACAAAGCAACAACAAGTACGC | |
| | GGCCAGCAGCTACCTGAGCCTGACGCCTGA | |
| | GCAGTGGAAGTCCCACAGAAGCTACAGCTG | |
| | CCAGGTCACGCATGAAGGGAGCACCGTGGA | |
| | GAAGACAGTGGCCCCTACAGAATGTTCATAG | |
| | GTTCTCAACCCTCACCCCCCACCACGGGAGA | |
| | CTAGAGCTGCAGGATCCCAGG | |

9F11

| | | |
|---|---|---|
| SEQ ID NO: 96 | CGGATCAACCCTAAGAGTGGTAACACAAACT | CDRH2 |
| | ATGCACAGAAATTTCAGGGC | |
| SEQ ID NO: 97 | GTTAAGATAGTAGTGGTAAGGAGCACGCCAA | CDRH3 |
| | CCCTCTACTACCTTGACTAC | |
| SEQ ID NO: 98 | ACTGGATCCAGCAGTGATGTTGGGACTTATG | CDRL1 |
| | ACCTTGTCTCC | |
| SEQ ID NO: 99 | GAGGGCCATAAGCGGCCCTCA | CDRL2 |
| SEQ ID NO: 100 | CAGGTGCAGCTGGTGCAGTCTGGGGCTGAG | VH |
| | GTGAAGAAACCTGGGGCCTCAGTGAAGGTC | |
| | TCCTGCAAGACTTCTGGATACACCTTCACCGA | |
| | CTACTATTTACACTGGGTGCGACAGGCCCCT | |
| | GGACAAGGGCTTGAGTGGATGGGACGGATC | |
| | AACCCTAAGAGTGGTAACACAAACTATGCAC | |
| | AGAAATTTCAGGGCCGGGTCACCGTGACCA | |
| | GGGACACGTCCATCAGCACAGCCTACATGGA | |
| | GCTGAGCAGGTTGAGATCTGACGACACGGC | |
| | CGTGTATTATTGTGCGAGAGTTAAGATAGTA | |
| | GTGGTAAGGAGCACGCCAACCCTCTACTACC | |
| | TTGACTACTGGGGCCAGGGAACCCTGGTCAC | |
| | CGTGTCCTCA | |
| SEQ ID NO: 101 | CAGTCTGCCCTGACTCAGCCTGCCTCCGTGT | VL |
| | CTGGGTCTCCTGGACAGTCGATCACCATCTC | |
| | CTGCACTGGATCCAGCAGTGATGTTGGGACT | |
| | TATGACCTTGTCTCCTGGTACCAACAGCACCC | |
| | AGGCAAAGCCCCCAAGGTCATGATTTATGAG | |
| | GGCCATAAGCGGCCCTCAGGGGTTTCTAATC | |
| | GCGTCTCTGGCTCCAAGTCTGGCAACACGGC | |
| | CTCCCTGACAATCTCCGGGCTCCAGGCTGAG | |
| | GACGAGGCTGATTATTACTGCTGCTCATATGC | |
| | AGGTACTAGCACCTTGGTGTTCGGCGGAGG | |
| | GACCAAGGTGACCGTCCTA | |

15D7

| | | |
|---|---|---|
| SEQ ID NO: 102 | AACTATGCTATGCAC | CDRH1 |
| SEQ ID NO: 103 | GCTATGAGTAGTAATGGGGGTAGCACATACT | CDRH2 |
| | ACGGAGACTCCGTGAAGGGC | |
| SEQ ID NO: 104 | CGGGGTAGAAAGGCGCCTTCGCTTTACTACT | CDRH3 |
| | TTGACTGC | |
| SEQ ID NO: 105 | AGGGCCAGTCAGAGTGTTAGCAGCAACTTAG | CDRL1 |
| | CC | |
| SEQ ID NO: 106 | GGTGCATCCACCAGGGCCACC | CDRL2 |
| SEQ ID NO: 107 | CAGCAGTATAATAACTGGCCTCCGTGGACC | CDRL3 |
| SEQ ID NO: 108 | GAGGTGCAGCTGGTGGAGTCTGGGGGAGG | VH |
| | CTTGGTCCAGCCTGGGGGGTCCCTGAGACTC | |
| | TCCTGTTCAGCCTCTGGATTCACCTTCGGTAA | |
| | CTATGCTATGCACTGGGTCCGCCAGGCTCCA | |
| | GGGAAGGGACTGGAATATGTTTCAGCTATGA | |
| | GTAGTAATGGGGGTAGCACATACTACGGAG | |
| | ACTCCGTGAAGGGCAGATTCACCATCTCCAG | |
| | AGACAATTCCAAGAACACGTTGTATCTTCAAA | |
| | TGAGCAGTCTGAGAGCTGAGGACACGGCTG | |
| | TTTATTACTGTGTGACGCGGGGTAGAAAGGC | |
| | GCCTTCGCTTTACTACTTTGACTGCTGGGGCC | |
| | CGGGAACCCTGGTCACCGTCTCCTCA | |
| SEQ ID NO: 109 | GAAATTGTGTTGACACAGTCTCCAGCCACCCT | VL |
| | GTCTGTGTCTCCAGGGGAAAGAGCCACCCTC | |
| | TCCTGCAGGGCCAGTCAGAGTGTTAGCAGCA | |
| | ACTTAGCCTGGTACCAGCAGAAACCTGGCCA | |
| | GGCTCCCAGGCTCCTCATCTATGGTGCATCC | |
| | ACCAGGGCCACCGGTATCCCAGCCAGGTTCA | |
| | GTGGCAGTGGGTCTGGGACAGAGTTCACTCT | |
| | CACCATCAGCAGCCTGCAGTCTGAAGATTTT | |
| | GCAGTTTATTACTGTCAGCAGTATAATAACTG | |
| | GCCTCCGTGGACCTTCGGCCAAGGGACACG | |
| | ACTGGAGATTAAA | |
| SEQ ID NO: 110 | MSEYIRVTEDENDEPIEIPSEDDGTVLLSTVTAQFP | TDP-43 variant |
| | GACGLRYRNPVSQCMRGVRLVEGILHAPDAG | |
| | WGNLVYVVNYPKDNKRKMDETDASSAVKVKR | |
| | AVQKTSDLIVLGLPWKTTEQDLKEYFSTFGEVLM | |
| | VQVKKDLKTGHSKGFGFVRFTEYETQVKVMSQ | |
| | RHMIDGRWCDCKLPNSKQSQDEPLRSKVFVGR | |
| | CTEDMTEDELREFFSQYGDVMDVFIPKPFRAFAF | |
| | VTFADDQIAQSLCGEDLIIKGISVHISNAEPKHNS | |
| | NRQLERSGRFGGNPGGFGNQGGFGNSRGGG | |
| | AGLGNNQGSNMGGGMNFGAFSINPAMMAAA | |
| | QAALQSSWGMMGMLASQQNQSGPSGNNQN | |
| | QGNMQREPNQAFGSGNNSYSGSNSGAAIGW | |
| | GSASNAGSGSGFNGGFGSSMDSKSSGWGM | |
| SEQ ID NO: 111 | QVQLVQSGAEVKKPGASVKVSCKASGYTFTGYY | 31F3 Hf VH |
| | LHWVRQAPGQGLEWMGRINPGSGGTKYAQKF | |
| | QGRVTMTRDTSISTAYMELSRLRSDDTAVYYCA | |
| | RYQVYGERGRYMDVWGKGTTVTVSS | |
| SEQ ID NO: 112 | IQSALTQPASVSGSPGQSITISCTGTSSDVGTYDL | 31F3 Lf VL |
| | VSWYQQHPGKAPKLMIYEDNKRPSGVSNRFSG | |
| | SKSGNTASLTISGLQAEDEADYYCCSYAAISTLIFG | |
| | SGTKVTVL | |
| SEQ ID NO: 113 | QVQLVQSGAEVKRPGASVKVSCKASGYTFTGYY | 31F3 Hg VH |
| | LHWVRQAPGQGLEWMGRINPGSGGTKYAQKF | |
| | QGRVTMTRDTSISTAYMELSRLRSDDTAVYYCA | |
| | RYQVYGERGRYMDVWGKGTTVTVSS | |
| SEQ ID NO: 114 | QSALTQPASVSGSPGQSITISCTGTSSDVGTYDL | 31F3 Lg VL |
| | VSWYQQHPGKAPKLIIYEDNKRPSGVSNRFSGS | |
| | KSGNTASLTISGLQAEDEADYYCCSYAAISTLIFGS | |
| | GTKVTVL | |

1. An antibody, or an antigen-binding fragment thereof, binding to a phosphorylated C-terminal domain or fragment of TAR-DNA-binding protein 43 kDa (TDP-43; SEQ ID NO: 1) comprising amino acids 391-414 of SEQ ID NO: 1, wherein the serine residues at positions 403 and/or 404 of SEQ ID NO: 1 are phosphorylated.

2. The antibody, or an antigen-binding fragment thereof, according to claim 1, wherein the antibody or the antigen-binding fragment does not specifically bind to a phosphorylated C-terminal domain or fragment of TDP-43 (SEQ ID NO: 1) comprising amino acids 391-414 of SEQ ID NO: 1, wherein the serine residues at positions 409 and/or 410 of SEQ ID NO: 1 (but not the serine residues at positions 403 and 404 of SEQ ID NO: 1) are phosphorylated.

3. The antibody, or an antigen-binding fragment thereof, according to any one of the previous claims, wherein the antibody or the antigen-binding fragment does not bind to non-pathological TDP-43.

4. The antibody, or an antigen-binding fragment thereof, according to any one of the previous claims, wherein the antibody or the antigen-binding fragment binds to high-molecular weight aggregates of TDP-43.

5. The antibody, or an antigen-binding fragment thereof, according to any one of the previous claims, wherein the antibody or the antigen-binding fragment binds to C-terminal fragments of TDP-43.

6. The antibody, or an antigen-binding fragment thereof, according to any one of the previous claims, wherein the antibody or the antigen-binding fragment binds to cytoplasmic or neuritic TDP-43 in human brain or spinal cord tissue.

7. The antibody, or an antigen-binding fragment thereof, according to any one of the previous claims, wherein the antibody or the antigen-binding fragment decreases TDP-43 aggregation.

8. The antibody, or an antigen-binding fragment thereof, according to any one of the previous claims, wherein the antibody or the antigen-binding fragment comprises:
(i) a CDRH1 according to General Formula I:
X₁YYX₂H (I)
wherein
X₁ may be any amino acid; preferably X₁ is a small amino acid selected from the group consisting of G, A, S, C, N, D, T, V and P; more preferably X₁ is G or D; and
X₂ may be any amino acid; preferably X₂ is a nonpolar amino acid selected from the group consisting of A, C, G, I, L, M, F, P, W and V; more preferably X₂ is M or L; even more preferably X₂ is L;
(ii) a CDRH2 according to General Formula II:
RINPX₁X₂GX₃X₄X₅YAQX₆FQG (II)
wherein
X₁ may be any amino acid; preferably X₁ is N, K or G;
X₂ may be any amino acid; preferably X₂ is a small amino acid selected from the group consisting of G, A, S, C, N, D, T, V and P; more preferably X₂ is S or A; particularly preferably X₂ is S;
X₃ may be any amino acid; preferably X₃ is a small amino acid selected from the group consisting of G, A, S, C, N, D, T, V and P; more preferably X₃ is G or N;
X₄ may be any amino acid; preferably X₄ is a small amino acid selected from the group consisting of G, A, S, C, N, D, T, V and P, or a hydrophobic amino acid selected from the group consisting of A, T, P, C, V, R, F, Y, W, M, I and L; more preferably X₄ is a small hydrophobic amino acid selected from the group consisting of A, T, P, C, and V; even more preferably X₄ is T, V or A; particularly preferably X₄ is T;
X₅ may be any amino acid; preferably X₅ is a polar amino acid selected from the group consisting of N, K, R, Q, S, T, D, E, H, Y and W; more preferably X₅ is N, K, R or Q; particularly preferably X₅ is K or N; and
X₆ may be any amino acid; preferably X₆ is a polar amino acid selected from the group consisting of N, K, R, Q, S, T, D, E, H, Y and W; more preferably X₆ is K, R or Q; particularly preferably X₆ is K;
(iii) a CDRH3 according to SEQ ID NO: 5 or according to General Formula III:
VX₁IVVLRSTPTLYYX₂DY (III)
wherein
X₁ may be any amino acid; preferably X₁ is M, K or R; more preferably X₁ is K or R; even more preferably X₁ is K; and
X₂ may be any amino acid; preferably X₂ is a hydrophobic amino acid selected from the group consisting of A, T, P, C, V, R, F, Y, W, M, I and L; more preferably X₂ is F or L;
(iv) a CDRL1 according to General Formula IV:
TGX₁SSDVGX₂YX₃LVS (IV)
wherein
X₁ may be any amino acid; preferably X₁ is a small amino acid selected from the group consisting of G, A, S, C, N, D, T, V and P or a polar amino acid selected from the group consisting of N, K, R, Q, S, T, D, E, H, Y and W; more preferably X₁ is a small polar amino acid selected from the group consisting of S, T, D or N; even more preferably X₁ is S or T;
X₂ may be any amino acid; preferably X₂ is a small amino acid selected from the group consisting of G, A, S, C, N, D, T, V and P or a polar amino acid selected from the group consisting of N, K, R, Q, S, T, D, E, H, Y and W; more preferably X₂ is a small polar amino acid selected from the group consisting of S, T, D or N; even more preferably X₂ is T or S; particularly preferably X₂ is T; and
X₃ may be any amino acid; preferably X₃ is a small amino acid selected from the group consisting of G, A, S, C, N, D, T, V and P or a polar amino acid selected from the group consisting of N, K, R, Q, S, T, D, E, H, Y and W; more preferably X₃ is a small polar amino acid selected from the group consisting of S, T, D or N; even more preferably X₃ is D or N; particularly preferably X₃ is D;
(v) a CDRL2 according to General Formula V:
EX₁X₂X₃RPS (V)
wherein
X₁ may be any amino acid; preferably X₁ is D, L, Q, A or G or a small amino acid selected from the group consisting of G, A, S, C, N, D, T, V and P; more preferably X₁ is D or G;
X₂ may be any amino acid; preferably X₂ is a polar amino acid selected from the group consisting of N, K, R, Q, S, T, D, E, H, Y and W; more preferably X₂ is N, S or H; and
X₃ may be any amino acid; preferably X₃ is a polar amino acid selected from the group consisting of N, K, R, Q, S, T, D, E, H, Y and W; more preferably X₃ is K, R or Q; particularly preferably X₃ is K;
and
(vi) a CDRL3 according to General Formula VI:
X₁SYAX₂X₃STX₄X₅ (VI)
wherein
X₁ may be any amino acid; preferably X₁ is C, S, Q, or G or a small amino acid selected from the group consisting of G, A, S, C, N, D, T, V and P; more preferably X₁ is C;
X₂ may be any amino acid; preferably X₂ is a small amino acid selected from the group consisting of G, A, S, C, N, D, T, V and P or an aliphatic non-polar amino acid selected from the group consisting of A, G, I, L and V; more preferably X₂ is a small aliphatic non-polar amino acid selected from the group consisting of A, G and V; even more preferably X₂ is A or G;
X₃ may be any amino acid; preferably X₃ is I, S or T; more preferably X₃ is I or T;
X₄ may be any amino acid; preferably X₄ is a hydrophobic amino acid selected from the group consisting of A, T, P, C, V, R, F, Y, W, M, I and L; more preferably X₄ is L or W; even more preferably X₄ is L; and
X₅ may be any amino acid; preferably X₅ is an aliphatic non-polar amino acid selected from the group consisting of A, G, I, L and V; more preferably X₅ is I or V.

9. The antibody, or an antigen-binding fragment thereof, according to any one of the previous claims, wherein the antibody or the antigen-binding fragment comprises:
(i) a CDRH1 according to General Formula I:
X₁YYX₂H (I)
wherein
X₁ is G or D; and
X₂ is L;
(ii) a CDRH2 according to General Formula II:
RINPX₁X₂GX₃X₄X₅YAQX₆FQG (II)
wherein
X₁ is N, K or G;
X₂ is S;
X₃ is G or N;
X₄ is T;
X₅ is K or N; and
X₆ is K,
(iii) a CDRH3 according to SEQ ID NO: 5 or according to General Formula III:
VX₁IVVLRSTPTLYYX₂DY (III)
wherein
X₁ is K; and
X₂ is F or L.
(iv) a CDRL1 according to General Formula IV:
TGX₁SSDVGX₂YX₃LVS (IV)
wherein
X₁ is S or T;
X₂ is T; and
X₃ is D;
(v) a CDRL2 according to General Formula V:
EX₁X₂X₃RPS (V)
wherein
X₁ is D or G;
X₂ is N, S or H; and
X₃ is K;
and
(vi) a CDRL3 according to General Formula VI:
X₁SYAX₂X₃STX₄X₅ (VI)
wherein
X₁ is C;
X₂ is A or G;
X₃ is I or T;
X₄ is L; and
X₅ is I or V.

10. The antibody, or an antigen-binding fragment thereof, according to any one of the previous claims, wherein the antibody or the antigen-binding fragment comprises:
(i) CDRH1, CDRH2, CDRH3, CDRL1, CDRL2 and CDRL3 sequences according to SEQ ID NOs 3-8, respectively; or
(ii) CDRH1, CDRH2, CDRH3, CDRL1, CDRL2 and CDRL3 sequences according to SEQ ID NOs 13, 14, 15, 6, 16 and 17, respectively.

11. The antibody, or an antigen-binding fragment thereof, according to any one of the previous claims, wherein the antibody or the antigen-binding fragment comprises:
(i) CDRH1, CDRH2, CDRH3, CDRL1, CDRL2 and CDRL3 sequences according to SEQ ID NOs 3-8, respectively;
(ii) CDRH1, CDRH2, CDRH3, CDRL1, CDRL2 and CDRL3 sequences according to SEQ ID NOs 13, 14, 15, 6, 16 and 17, respectively;
(iii) CDRH1, CDRH2, CDRH3, CDRL1, CDRL2 and CDRL3 sequences according to SEQ ID NOs 13, 22, 23, 24, 25 and 17, respectively; or
(iv) CDRH1, CDRH2, CDRH3, CDRL1, CDRL2 and CDRL3 sequences according to SEQ ID NOs 28-33, respectively.

12. The antibody, or an antigen-binding fragment thereof, according to any one of the previous claims, wherein the antibody or the antigen-binding fragment comprises (i) a heavy chain variable region comprising an amino acid sequence having at least 70% identity to SEQ ID NO: 9 and a light chain variable region comprising the amino acid sequence having at least 70% identity to SEQ ID NO: 10; or (ii) a heavy chain variable region comprising an amino acid sequence having at least 70% identity to SEQ ID NO: 18 and a light chain variable region comprising the amino acid sequence having at least 70% identity to SEQ ID NO: 19; or (iii) a heavy chain variable region comprising an amino acid sequence having at least 70% identity to SEQ ID NO: 26 and a light chain variable region comprising the amino acid sequence having at least 70% identity to SEQ ID NO: 27; or (iv) a heavy chain variable region comprising an amino acid sequence having at least 70% identity to SEQ ID NO: 34 and a light chain variable region comprising the amino acid sequence having at least 70% identity to SEQ ID NO: 35.

13. The antibody, or an antigen-binding fragment thereof, according to any one of the previous claims, wherein the antibody or the antigen-binding fragment comprises (i) a heavy chain variable region comprising an amino acid sequence having at least 70% identity to SEQ ID NO: 9 and a light chain variable region comprising an amino acid sequence having at least 70% identity to SEQ ID NO: 10; or (ii) a heavy chain variable region comprising an amino acid sequence having at least 70% identity to SEQ ID NO: 18 and a light chain variable region comprising the amino acid sequence having at least 70% identity to SEQ ID NO: 19.

14. The antibody, or an antigen-binding fragment thereof, according to any one of the previous claims, wherein the antibody or the antigen-binding fragment comprises a heavy chain variable region comprising an amino acid sequence having at least 70% identity to any one of SEQ ID NO: 9, 111 and 113; and a light chain variable region comprising an amino acid sequence having at least 70% identity to SEQ ID NO: 10, 112 or 114.

15. The antibody, or an antigen-binding fragment thereof, according to any one of the previous claims, wherein the antibody or the antigen-binding fragment comprises (i) a heavy chain variable region comprising an amino acid sequence according to SEQ ID NO: 9 and a light chain variable region comprising the amino acid sequence according to SEQ ID NO: 10; or (ii) a heavy chain variable region comprising an amino acid sequence according to SEQ ID NO: 18 and a light chain variable region comprising the amino acid sequence according to SEQ ID NO: 19; or (iii) a heavy chain variable region comprising an amino acid sequence according to SEQ ID NO: 26 and a light chain variable region comprising the amino acid sequence according to SEQ ID NO: 27; or (iv) a heavy chain variable region comprising an amino acid sequence according to SEQ ID NO: 34 and a light chain variable region comprising the amino acid sequence according to SEQ ID NO: 35.

16. The antibody, or an antigen-binding fragment thereof, according to any one of the previous claims, wherein the antibody or the antigen-binding fragment comprises (i) a heavy chain variable region comprising an amino acid sequence according to SEQ ID NO: 9 and a light chain variable region comprising an amino acid sequence according to SEQ ID NO: 10; or (ii) a heavy chain variable region comprising an amino acid sequence according to SEQ ID NO: 18 and a light chain variable region comprising the amino acid sequence according to SEQ ID NO: 19.

17. The antibody, or an antigen-binding fragment thereof, according to any one of the previous claims, wherein the antibody or the antigen-binding fragment comprises a heavy chain variable region comprising an amino acid sequence according to any one of SEQ ID NO: 9, 111 and 113; and a light chain variable region comprising an amino acid sequence according to SEQ ID NO: 10, 112 or 114.

18. The antibody, or an antigen-binding fragment thereof, according to any one of the previous claims, wherein the antibody or the antigen-binding fragment thereof is a human antibody.

19. The antibody, or an antigen-binding fragment thereof, according to any one of the previous claims, wherein the antibody, or an antigen-binding fragment thereof, is a monoclonal antibody.

20. The antibody of any one according to the previous claims, wherein the antibody comprises an Fc moiety.

21. The antibody according to any one of the previous claims, wherein the antibody is of the IgG type.

22. The antibody according to claim 21, wherein the antibody is of the IgG1 or IgG4 type.

23. The antibody, or an antigen-binding fragment thereof, according to any one of the previous claims, wherein the antibody, or the antigen-binding fragment thereof, is purified.

24. The antibody, or an antigen-binding fragment thereof, according to any one of the previous claims, wherein the antibody, or the antigen-binding fragment thereof, is a single-chain antibody.

25. The antibody, or an antigen-binding fragment thereof, according to claim 24, wherein the antibody, or the antigen-binding fragment thereof, is an scFv.

26. The antibody, or an antigen-binding fragment thereof, according to any one of claims 1 to 23, wherein the antibody, or the antigen-binding fragment thereof, is selected from Fab, Fab′, F(ab′)2 and Fv.

27. The antibody, or an antigen-binding fragment thereof, according to any one of the previous claims, wherein the antibody, or the antigen-binding fragment thereof, is an intrabody.

28. The antibody, or an antigen-binding fragment thereof, according to any one of the previous claims for use as a medicament.

29. The antibody, or an antigen-binding fragment thereof, for use according to claim 28 in prophylaxis or treatment of TDP-43 proteinopathy.

30. A nucleic acid molecule comprising a polynucleotide encoding the antibody, or an antigen-binding fragment thereof, according to any one of claims 1 to 27.

31. The nucleic acid molecule according to claim 30, wherein the polynucleotide encoding the antibody, or an antigen-binding fragment thereof, is codon-optimized.

32. The nucleic acid molecule according to claim 30 or 31 comprising a nucleic acid sequence as set forth in any one of SEQ ID NOs 76-109; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.

33. The nucleic acid molecule according to any one of claims 30-32, wherein the encoded antibody, or an antigen-binding fragment thereof, is an intrabody.

34. A combination of a first and a second nucleic acid molecule, wherein the first nucleic acid molecule comprises a polynucleotide encoding the heavy chain of the antibody, or an antigen-binding fragment thereof, according to any one of claims 1 to 27; and the second nucleic acid molecule comprises a polynucleotide encoding the corresponding light chain of the same antibody, or the same antigen-binding fragment thereof.

35. The combination of nucleic acid molecules according to claim 34, wherein one or both of the polynucleotides encoding the heavy and/or light chain(s) of the antibody, or an antigen-binding fragment thereof, is/are codon-optimized.

36. The combination of nucleic acid molecules according to claim 34 or 35 comprising a nucleic acid sequence as set forth in any one of SEQ ID NOs 76-109; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.

37. A vector comprising the nucleic acid molecule of any one of claims 30 to 33 or the combination of nucleic acid molecules of any one of claims 34 to 36.

38. A combination of a first and a second vector, wherein the first vector comprises a first nucleic acid molecule as defined in any one of claims 34 to 36 and the second vector comprises the corresponding second nucleic acid molecule as defined in any one of claims 34 to 36.

39. A host cell expressing the antibody, or an antigen-binding fragment thereof, according to any one of claims 1 to 27, or comprising the vector according to claim 37 or the combination of vectors of claim 38.

40. A method for preparing the antibody, or an antigen-binding fragment thereof, according any one of claims 1 to 27, or immunoglobulin chain(s) thereof, said method comprising
(i) culturing the host cell according to claim 39; and
(ii) isolating the antibody or immunoglobulin chain(s) thereof from the culture.

41. An immunoconjugate comprising the antibody, or an antigen-binding fragment thereof, according to any one of claims 1 to 27 and a detectable label and/or a transport moiety.

42. A composition comprising the antibody, or an antigen-binding fragment thereof, according to any one of claims 1 to 27, the nucleic acid according to any one of claims 30 to 33, the combination of nucleic acids according to any one of claims 34 to 36, the vector according to claim 37, the combination of vectors according to claim 38, the cell according to claim 39 or the immunoconjugate according to claim 41.

43. The composition according to claim 42, which is a pharmaceutical composition and, optionally, further comprises a pharmaceutically acceptable excipient, diluent or carrier.

44. The composition according to claim 42, which is a diagnostic composition.

45. The antibody, or an antigen-binding fragment thereof, according to any one of claims 1 to 27, the nucleic acid according to any one of claims 30 to 33, the combination of nucleic acids according to any one of claims 34 to 36, the vector according to claim 37, the combination of vectors according to claim 38, the cell according to claim 39, the immunoconjugate according to claim 41, or the pharmaceutical composition according to claim 43 for use as a medicament; optionally in the prophylaxis or treatment of a TDP-43 proteinopathy.

46. Use of the antibody, or an antigen-binding fragment thereof, according to any one of claims 1 to 27 or of the immunoconjugate according to claim 41 in (in-vitro) diagnosis of a TDP-43 proteinopathy.

47. Use of the antibody, or an antigen-binding fragment thereof, according to any one of claims 1 to 27 or of the immunoconjugate according to claim 41 in a method for detecting pathological TDP-43.

48. Use of the antibody, or an antigen-binding fragment thereof, according to any one of claims 1 to 27 or of the immunoconjugate according to claim 41 in the testing of an immunogenic composition comprising a phosphorylated C-terminal domain or fragment of TDP-43 (SEQ ID NO: 1) comprising amino acids 391-414 of SEQ ID NO: 1, wherein the serine residues at positions 403 and 404 of SEQ ID NO: 1 are phosphorylated, in particular for monitoring the quality of the immunogenic composition.

49. Use of the antibody, or an antigen-binding fragment thereof, according to any one of claims 1 to 27, the nucleic acid according to any one of claims 30 to 33, the combination of nucleic acids according to any one of claims 34 to 36, the vector according to claim 37, the combination of vectors according to claim 38, the cell according to claim 39, the immunoconjugate according to claim 41, or the pharmaceutical composition according to claim 43 in the manufacture of a medicament for prophylaxis, treatment or attenuation of a TDP-43 proteinopathy.

50. A method of reducing a TDP-43 proteinopathy, or lowering the risk of a TDP-43 proteinopathy, comprising: administering to a subject in need thereof, a therapeutically effective amount of the antibody, or an antigen-binding fragment thereof, according to any one of claims 1 to 27, the nucleic acid according to any one of claims 30 to 33, the combination of nucleic acids according to any one of claims 34 to 36, the vector according to claim 37, the combination of vectors according to claim 38, the cell according to claim 39, the immunoconjugate according to claim 41, or the pharmaceutical composition according to claim 43.

51. A kit comprising one or more containers containing one or more of
(i) the antibody, or an antigen-binding fragment thereof, according to any one of claims 1 to 27,
(ii) the nucleic acid molecule(s) according to any one of claims 30 to 36,
(iii) the vector(s) according to claim 37 or 38,
(iv) the cell according to claim 39,
(v) the immunoconjugate according to claim 41; and/or
(vi) the composition according to any one of claims 42 to 44.
